(19) United States
(12) Patent Application Publication
(10) Pub. No.: US 2011/0098298 A1
(43) Pub. Date: Apr. 28, 2011

(54) New Pyridin-3-Amine Derivatives

(76) Inventors: Bernat Vidal Juan, Sant Cebria de Vallalta (ES); Juan Francisco Caturla Javaloyes, Rubi (ES); Wenceslao Lumeras Amador, Terrassa (ES); Laura Vidal Gispert, Barcelona (ES)

(21) Appl. No.: 12/279,843

(22) PCT Filed: Feb. 12, 2007
(86) PCT No.: PCT/EP07/01174

§ 371 Date: Sep. 22, 2008

(30) Foreign Application Priority Data
Feb. 20, 2006 (ES) P200600396

(51) Int. Cl. A61K 31/5377; C07D 213/89; C07D 413/12; A61K 31/44

(52) U.S. Cl. 514/235.5; 546/310; 514/352; 544/131

(57) ABSTRACT

This invention is directed to new inhibitors of the p38 mitogen-activated protein kinase having the general formula (I)

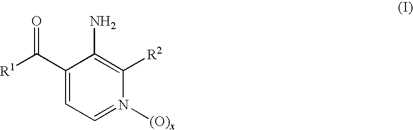

to processes for their preparation; to pharmaceutical compositions comprising them; and to their use in therapy.

The present invention relates to new inhibitors of the p38 mitogen-activated protein kinase.

MAP kinases are evolutionary conserved enzymes translating membrane signals into gene expression responses. In mammals, four MAPK families can be distinguished: extracellular signal-related kinases (ERK1/2), Jun amino terminal kinases (JNK1/2/3), p38 proteins (alpha, beta, gamma and delta) and ERK5. The regulation of these proteins is exerted by a three-tier cascade composed of MAPK, MAPK kinase, and MAPK kinase kinase.

p38 MAPK was originally identified as the target of CSAIDs (cytokine suppressive anti-inflammatory drugs), having a central role in the signal transduction pathway leading to the production of TNF-alpha and other cytokines (Lee et al, 1984). p38 is activated by phosphorylation in Thr and Tyr by either MKK3, MKK4, or MKK6 (Kyriakis and Avruch, 2001) in response to stress and pro-inflammatory stimuli. In turn, p38 phosphorylates its effectors in Ser and Thr residues, namely protein kinases phosphatases and transcription factors, such as ATF-2, MEF2, MAPKAPK2, MSK1/2 or MNK1/2. Altogether this activation cascade results in control of gene expression through four different mechanisms: transcription factor activation; mRNA stabilization; mRNA translation; and histone phosphorylation at NF-kB binding sites in chromatin (Shi and Gaestel, 2002; Sacanni et al, 2001).

There are four different p38 isoforms encoded by separate genes: p38 alpha, beta, gamma and delta, each one showing a distinct tissue expression pattern. As assessed by mRNA and protein levels (Beardmore et al, 2005; Wang et al, 1997), p38 alpha and beta are ubiquitously expressed, with p38 beta expression being more relevant in CNS tissues (brain, cortex, cerebellum, hippocampus, etc). The expression of p38 gamma is more prominent in skeletal muscle while p38 delta localizes mainly in heart, kidney, lung and adrenal gland. At the cellular level, p38 alpha and delta seem to be the most relevant isoforms in immune cells (monocytes, macrophages, neutrophils and T cells) (Hale et al, 1999). Pharmacological inhibition with specific p38alpha/beta inhibitors as well as gene targeting studies have indicated that p38alpha is the isoform regulating inflammatory responses most probably through its downstream substrate MAPKAP-K2 (Kotlyarov et al, 1999). Likewise, this isoform is necessary in early embryonic development as p38alpha KO (knock-out) mice die in embryonic day 12.5 due to placental insufficiency and vascular defects (Allen et al, 2000; Tamura et al, 2000; Adams et al, 2000), a phenotype that is also reproduced in the MKK3/MKK6 double KO mice (Brancho et al, 2003). In contrast, p38 beta, gamma and delta knock-out mice do not show any developmental deficiencies (Beardmore et al 2005; Sabio et al, 2005). p38 beta KO mice appear to respond similarly to pro-inflammatory stimuli (LPS) as wild type controls, indicating that this isoform does not have a role in inflammation (Beardmore et al 2005).

The contribution of the p38MAPK pathway to inflammation has been studied both in vitro and in vivo by employing different chemical series of p38 inhibitors (Pargellis and Regan, 2003; Kumar et al, 2003). The most widely used inhibitor molecule, SB203580, is, in fact, a dual p38alpha/beta inhibitor. Inhibition of p38 abrogates the release of TNF-alpha as well as other pro-inflammatory cytokines like IL-1, IL-6, and IL-8, in PBMC, whole blood, or the human monocytic cell line THP-1.

By virtue of the involvement of p38 in TNFalpha production, inhibitors of p38 have been tested in animal models of diseases in which TNFalpha has a pathophysiological role. p38 inhibition decreases murine collagen-induced arthritis and rat adjuvant-induced arthritis severity (Pargellis and Regan, 2003). Furthermore, p38 inhibitors also improve bone resorption in animal models of arthritis, probably due to the implication of p38 MAPK in the differentiation of osteoclasts. p38 inhibition attenuates the inflammatory response in a murine model of Crohn's disease and diminishes TNF-alpha production in human Crohn's disease patient biopsies (Hollenbach et al 2005; Waetzig et al, 2002). Due to the exclusive usage of the p38 pathway by neutrophils, p38 has also been considered a target for chronic obstructive pulmonary disease (COPD) (Nick et al, 2002). p38 inhibition reduces neutrophilia, inflammatory cytokines, MMP-9 and fibrosis in lung (Underwood et al, 2000). In skin models of irradiation, inhibition of p38 protects the epidermis against acute ultraviolet radiation exposure by blocking apoptosis and inflammatory responses (Hildesheim et al, 2004). p38 inhibition also reverses hematopoietic defects in bone marrow from patients with myelodysplastic syndromes, in which TNF-alpha overproduction has a pathophysiological role (Katsoulidis et al, 2005).

In hematopoietic malignancies, a study has shown that p38 inhibitors can block the proliferation of multiple myeloma cells by inhibiting the production of IL-6 and VEGF in bone marrow stromal cells (Hideshima et al, 2002).

p38 is involved in key cellular mechanisms such as apoptosis, fibrosis and cellular hypertrophy, which are common to cardiac and vascular pathologies. Pharmacological inhibition of p38 has proven useful in improving ischemia-reperfusion injury, cerebral focal ischemia, acute coronary syndrome, chronic heart failure and post-myocardial infarction remodelling (See et al, 2004).

Experimental inhibition of p38 has been reported effective in reducing pain in animal models of neuropathy that rely on COX-2 expression and TNF-alpha production by glial cells (Schafers et al, 2003; Jin et al, 2003; Tsuda et al, 2004).

Therefore, the compounds of the invention may be useful in the prophylaxis or treatment of any disease or disorder in which p38 kinase plays a role including conditions caused by excessive or unregulated pro-inflammatory cytokine production including for example excessive or unregulated TNF, IL-1, IL-6 and IL-8 production in a human, or other mammal. The invention extends to such a use and to the use of the compounds for the manufacture of a medicament for treating such cytokine-mediated diseases or disorders. Further, the invention extends to the administration to a human an effective amount of a p38 inhibitor for treating any such disease or disorder.

Diseases or disorders in which p38 kinase plays a role either directly or via pro-inflammatory cytokines including the cytokines TNF, IL-1, IL-6 and IL-8 include without limitation autoimmune diseases, immune and inflammatory diseases, destructive bone disorders, neoplastic disorders, neurodegenerative disorders, viral diseases, infectious diseases, cardiovascular diseases, angiogenesis-related disorders, and pain-related disorders.

Autoimmune diseases which may be prevented or treated include but are not limited to rheumatoid arthritis, psoriatic arthritis, ankylosing spondilytis, Reiter's syndrome, fibromyalgia, inflammatory bowel disease such as ulcerative colitis and Crohn's disease, multiple sclerosis, diabetes, glomerulonephritis, systemic lupus erythematosus, scleroderma, chronic thyroiditis, Grave's disease, hemolytic anemia, autoimmune gastritis, autoimmune neutropenia, thrombocytopenia, autoimmune chronic active hepatitis, myasthenia gravis, or Addison's disease.

Immune and inflammatory diseases which may be prevented or treated include but are not limited to asthma, COPD, respiratory distress syndrome, acute or chronic pancreatitis, graft versus-host disease, Behcet syndrome, inflammatory eye conditions such as conjunctivitis and uveitis, psoriasis, contact dermatitis, atopic dermatitis, sarcoidosis, gout, pyresis, transplant rejection, allergic rhinitis, allergic conjunctivitis,

Cardiovascular diseases which may be prevented or treated include but are not limited to ischemia-reperfusion injury, cerebral focal ischemia, acute coronary syndrome, congestive heart failure, cardiomyopathy, myocarditis, atherosclerosis, vasculitis and restenosis.

Destructive bone disorders which may be prevented or treated include but are not limited to osteoporosis, osteoarthritis and multiple myeloma-related bone disorder.

Neoplastic disorders which may be prevented or treated include but are not limited to solid tumors such as Kaposi's sarcoma, metastatic melanoma, and hematopoietic malignancies such as acute or chronic myelogenous leukemia and multiple myeloma.

Neurodegenerative diseases which may be prevented or treated include but are not limited to Parkinson's disease, Alzheimer's disease, neurodegenerative disease caused by traumatic injury, or Huntington's disease.

Viral diseases which may be prevented or treated include but are not limited to acute hepatitis infection (including hepatitis A, hepatitis B and hepatitis C), HIV infection, Epstein-Barr infection, CMV retinitis, SARS or avian influenza A infection.

Infectious diseases which may be prevented or treated include but are not limited to sepsis, septic shock, endotoxic shock, Gram negative sepsis, toxic shock syndrome, Shigellosis, or cerebral malaria.

Angiogenesis-related disorders which may be prevented or treated include but are not limited to hemangiomas, ocular neovascularization, macular degeneration or diabetic retinopathy.

Pain-related disorders which may be prevented or treated include but are not limited to neuropathic pain (such as diabetic neuropathy, post-herpetic or trigeminal neuralgia), cancer-related pain, chronic pain (such as lower back pain syndrome), and inflammatory pain.

Other miscellaneous diseases or disorders which may be prevented or treated include but are not limited to myelodysplastic syndrome, cachexia, endometriosis, acute skin injuries such as sunburn, and wound healing.

In view of the physiological effects mediated by inhibition of the p38 mitogen-activated protein kinase, several compounds have been recently disclosed for the treatment or prevention of rheumatoid arthritis, ischemia-reperfusion injury, cerebral focal ischemia, acute coronary syndrome, COPD, Crohn's disease, irritable bowel syndrome, adult respiratory distress syndrome, osteoporosis, neurodegenerative diseases such as Alzheimer's disease, rheumatoid spondylitis, psoriasis, atherosclerosis, osteoarthritis, multiple myeloma. See for example WO 99/01449, WO 00/63204, WO 01/01986, WO 01/29042, WO 02/046184, WO 02/058695, WO 02/072576, WO 02/072579, WO 03/008413, WO 03/033502, WO 03/087087, WO 03/097062, WO 03/103590, WO 2004/010995, WO 2004/014900, WO 2004/020438, WO 2004/020440, WO 2005/018624, WO 2005/032551, WO 2005/073219.

It has now been found that certain pyridin-3-amine derivatives are novel potent inhibitors of the p38 mitogen-activated protein kinase and can therefore be used in the treatment or prevention of these diseases.

Further objectives of the present invention are to provide a method for preparing said compounds; pharmaceutical compositions comprising an effective amount of said compounds; the use of the compounds in the manufacture of a medicament for the treatment of pathological conditions or diseases susceptible of being improved by inhibition of the p38 mitogen-activated protein kinase; and methods of treatment of pathological conditions or diseases susceptible to amelioration by inhibition of the p38 mitogen-activated protein kinase comprising the administration of the compounds of the invention to a subject in need of treatment.

Thus, the present invention is directed to new pyridin-3-amine derivatives of formula (I)

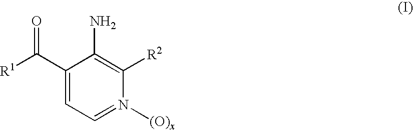

wherein:

R¹ represents a monocyclic or polycyclic, aryl or heteroaryl group optionally substituted by one, two or three substituents selected from the group consisting of halogen atoms, straight or branched C₁₋₆ alkyl, hydroxy, straight or branched C₁₋₆ alkoxy, —SH, straight or branched C₁₋₆ alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched C₁₋₆ alkyl group or R′ and R″ together with the atom to which they are attached form a non-aromatic heterocyclic group;
R² represents a cyclic group selected from the group consisting of aryl, heteroaryl, non-aromatic heterocyclic and carbocyclic groups, the cyclic groups being optionally substituted by one, two or three substituents selected from group consisting of halogen atoms, straight or branched C₁₋₆ alkyl, hydroxy, straight or branched C₁₋₆ alkoxy, —SH, straight or branched C₁₋₆ alkylthio, nitro, cyano, trifluoromethyl, trifluoromethoxy, —OR″, —NR′R″, —CO₂R′, —C(O)—NR′R″, —N(R′″)C(O)—R′, —N(R′″)—C(O)NR′R″, wherein R′, and R′″ each independently represents a hydrogen atom or a straight or branched C₁₋₆ alkyl group and R″ represents a group of formula —(CH₂)ₙ—Y-G wherein n is an integer from 1 to 3; Y is selected from the group consisting of direct bond, —O— and —NR^{IV}—; R^{IV} represents a hydrogen atom or a C₁₋₄ alkyl group; and G is a hydrogen atom, a C₁₋₆ alkyl group or a non-aromatic nitrogen-containing heterocyclic ring bound to the group Y through its nitrogen atom, or R′ and R″ together with the atom to which they are attached form a non-aromatic heterocyclic group;
   x has the value of zero or one;
   and pharmaceutically acceptable salts thereof.

To avoid any confusion, it is clarified that in the above formula when x has the value of zero the compounds of formula (I) are 3-amino-pyridin-4-yl ketones and when x has the value of one the compounds are 3-amino-1-oxido-pyridin-4-yl ketones.

As used herein the term lower alkyl embraces optionally substituted, linear or branched radicals having 1 to 8, preferably 1 to 6 and more preferably 1 to 4 carbon atoms. The substituents in said alkyl groups are selected from halogen atoms and hydroxy groups.

Examples include methyl, ethyl, n-propyl, i-propyl, n-butyl, sec-butyl and tert-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

As used herein, the term lower alkoxy embraces optionally substituted, linear or branched oxy-containing radicals each having alkyl portions of 1 to 8, preferably 1 to 6 and more preferably 1 to 4 carbon atoms. The substituents in said alkoxy groups are selected from halogen atoms and hydroxy groups.

Preferred alkoxy radicals include methoxy, ethoxy, n-propoxy, i-propoxy, n-butoxy, sec-butoxy, t-butoxy, trifluoromethoxy, difluoromethoxy, hydroxymethoxy, 2-hydroxyethoxy or 2-hydroxypropoxy.

As used herein, the term lower alkylthio embraces radicals containing an optionally substituted, linear or branched alkyl radicals of 1 to 8, preferably 1 to 6 and more preferably 1 to 4 carbon atoms. The substituents in said alkylthio groups are selected from halogen atoms and hydroxy groups.

Preferred optionally substituted alkylthio radicals include methylthio, ethylthio, n-propylthio, i-propylthio, n-butylthio, sec-butylthio, t-butylthio, trifluoromethylthio, difluoromethylthio, hydroxymethylthio, 2-hydroxyethylthio or 2-hydroxypropylthio.

As used herein, the term cyclic group embraces, unless otherwise specified, carbocyclic and heterocyclic radicals. The cyclic radicals can contain one or more rings. Carbocyclic radicals may be aromatic or alicyclic, for example cycloalkyl radicals. Heterocyclic radicals also include heteroaryl radicals.

As used herein, the term aromatic group embraces typically a 5- to 14-membered aromatic ring system, such as a 5- or 6-membered ring which may contain one or more heteroatoms selected from O, S and N. When no heteroatoms are present the radical is named aryl radical and when at least one heteroatom is present it is named heteroaryl radical. The aromatic radical can be monocyclic such as phenyl or pyridyl or polycyclic, such as naphthyl or quinolyl. When an aromatic radical or moiety carries 2 or more substituents, the substituents may be the same or different.

As used herein, the term aryl radical embraces typically a C₅-C₁₄ monocyclic or polycyclic aryl radical such as phenyl or naphthyl, anthranyl or phenanthryl. Phenyl is preferred. When an aryl radical carries 2 or more substituents, the substituents may be the same or different.

As used herein, the term heteroaryl radical embraces typically a 5- to 14-membered ring system comprising at least one heteroaromatic ring and containing at least one heteroatom selected from O, S and N. A heteroaryl radical may be a single ring or two or more fused rings wherein at least one ring contains a heteroatom.

Examples include pyridyl, pyrazinyl, pyrimidinyl, pyridazinyl, furyl, oxadiazolyl, oxazolyl, imidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl and pyrazolyl radicals. Pyridyl, thienyl, furanyl, pyridazinyl, pyrimidinyl and quinolyl radicals are preferred.

When a heteroaryl radical carries 2 or more substituents, the substituents may be the same or different.

As used herein, the term non-aromatic heterocyclic group embraces typically a non-aromatic, saturated or unsaturated C₃-C₁₀ carbocyclic ring, such as a 5, 6 or 7 membered radical, in which one or more, for example 1, 2, 3 or 4 of the carbon atoms preferably 1 or 2 of the carbon atoms are replaced by a heteroatom selected from N, O and S. Saturated heterocyclyl radicals are preferred. A heterocyclic radical may be a single ring or two or more fused rings wherein at least one ring contains a heteroatom. When a heterocyclyl radical carries 2 or more substituents, the substituents may be the same or different.

Examples of heterocyclic radicals include piperidyl, pyrrolidyl, pyrrolinyl, piperazinyl, morpholinyl, thiomorpholinyl, pyrrolyl, pyrazolinyl, pirazolidinyl, quinuclidinyl, triazolyl, pyrazolyl, tetrazolyl, cromanyl, isocromanyl, imidazolidinyl, imidazolyl, oxiranyl, azaridinyl, 4,5-dihydro-oxazolyl and 3-aza-tetrahydrofuranyl. Where a heterocyclyl radical carries 2 or more substituents, the substituents may be the same or different.

As used herein, some of the atoms, radicals, moieties, chains or cycles present in the general structures of the invention are “optionally substituted”. This means that these atoms, radicals, moieties, chains or cycles can be either unsubstituted or substituted in any position by one or more, for example 1, 2, 3 or 4, substituents, whereby the hydrogen atoms bound to the unsubstituted atoms, radicals, moieties, chains or cycles are replaced by chemically acceptable atoms, radicals, moieties, chains or cycles. When two or more substituents are present, each substituent may be the same or different.

As used herein, the term halogen atom embraces chlorine, fluorine, bromine or iodine atoms typically a fluorine, chlorine or bromine atom, most preferably chlorine or fluorine. The term halo when used as a prefix has the same meaning.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X—) is associated with the positive charge of the N atom. X— may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X— is preferably an anion selected from chloride, bromide, iodide, sulphate, nitrate, acetate, maleate, oxalate, succinate or trifluoroacetate. More preferably X— is chloride, bromide, trifluoroacetate or methanesulphonate.

As used herein, an N-oxide is formed from the tertiary basic amines or imines present in the molecule, using a convenient oxidising agent.

Preferred compounds of the present invention are the compounds of formula (I) wherein x has a value of 1.

In an embodiment of the present invention R¹ represents an optionally substituted monocyclic aryl or heteroaryl group.

In another embodiment of the present invention R¹ represents an optionally substituted phenyl group.

In another embodiment of the present invention R¹ represents a phenyl group which is unsubstituted or substituted by 1 or 2 groups selected from halogen atoms, methyl and methoxy groups, more preferably R¹ is substituted by 1 or 2 halogen atoms and most preferably substituted by 1 or 2 atoms selected from the group consisting of chlorine and fluorine.

In another embodiment of the present invention R² represents a 5-10 membered cyclic group which is unsubstituted or carries 1, 2 or 3 substituents selected from the group consisting of halogen atoms, C₁₋₄ alkyl groups, C₁₋₄ alkoxy groups, trifluoromethyl, trifluoromethoxy, —COOH, or groups of formula

—X—(CH₂)ₙ—Y-G

wherein X is selected from the group consisting of —C(O)NH—, —O— and —NH—; n is an integer from 1 to 3; Y is selected from the group consisting of direct bond, —O— and —NR^{IV}—;
R^{IV} represents a hydrogen atom or a C₁₋₄ alkyl group; and G is a non-aromatic nitrogen-containing heterocyclic ring bound to the group Y through its nitrogen atom.

In another embodiment of the present invention R² carries 1, 2 or 3 substituents and at least one of such substituents is in the ortho position with respect to the carbon atom through which R² is attached to the pyridine ring.

In still another embodiment of the present invention R² represents a 5-10 membered cyclic group comprising from 0 to 3 heteroatoms selected from nitrogen, oxygen and sulphur as part of the ring system.

In yet another embodiment of the present invention R² represents a group selected from the group consisting of phenyl, anthranyl, cyclohexyl, thienyl, furyl, pyridyl, benzodioxolyl and benzothienyl, all of them being optionally substituted.

Typically, R² represents a cyclic group which is unsubstituted or carries 1, 2 or 3 substituents selected from the group consisting of halogen atoms and groups C₁₋₄ alkoxy, trifluoromethyl, trifluoromethoxy, —COOH, —C(O)O—C₁₋₄-alkyl, C₁₋₄-alkyl, morpholinylethoxy, methoxyethoxy, [(2-morpholin-4-ylethyl)amino]carbonyl, [(2-methoxyethyl)amino]carbonyl, and {2-[(dimethylamino)ethyl]amino}carbonyl.

In still another embodiment the present invention is directed to a compound of formula (I) wherein R¹ represents a phenyl group which is substituted by 1 or 2 halogen atoms independently selected from the group consisting of chlorine and fluorine, R² represents a phenyl group which is substituted by 1 or 2 substituents selected from the group consisting of chlorine, fluorine, methyl, methoxy and hydroxy, and at least one of such substituents is in the ortho position with respect to the carbon atom through which R² is attached to the pyridine and x, preferably has a value of 1

Particular individual compounds of the invention include:
1. (3-Amino-2-phenylpyridin-4-yl)(phenyl)methanone
2. (3-Amino-1-oxido-2-phenylpyridin-4-yl)(phenyl)methanone
3. [3-Amino-2-(2-methylphenyl)pyridin-4-yl](phenyl)methanone
4. [3-Amino-2-(2-methylphenyl)-1-oxidopyridin-4-yl](phenyl)methanone
5. [3-Amino-2-(2,6-dichlorophenyl)pyridin-4-yl] (phenyl)methanone
6. [3-Amino-2-(2,6-dichlorophenyl)-1-oxidopyridin-4-yl](phenyl)methanone
7. [3-Amino-2-(2,6-difluoro-4-methoxyphenyl)pyridin-4-yl](phenyl)methanone
8. [3-Amino-2-(2,6-difluoro-4-methoxyphenyl)-1-oxidopyridin-4-yl](phenyl)methanone
9. [3-Amino-2-(4-chlorophenyl)pyridin-4-yl](phenyl)methanone
10. [3-Amino-2-(4-chlorophenyl)-1-oxidopyridin-4-yl](phenyl)methanone
11. (3-Amino-2-phenylpyridin-4-yl)(2,4-difluorophenyl)methanone
12. (3-Amino-1-oxido-2-phenylpyridin-4-yl)(2,4-difluorophenyl)methanone
13. [3-Amino-2-(2-hydroxyphenyl)pyridin-4-yl](2,4-difluorophenyl)methanone
14. [3-Amino-2-(2-hydroxyphenyl)-1-oxidopyridin-4-yl](2,4-difluorophenyl)methanone
15. 3-Amino-2-(2-methoxyphenyl)pyridin-4-yl](2,4-difluorophenyl)methanone
16. [3-Amino-2-(2-methoxyphenyl)-1-oxidopyridin-4-yl](2,4-difluorophenyl)methanone
17. {3-Amino-2-[2-(trifluoromethoxy)phenyl]pyridin-4-yl}(2,4-difluorophenyl)methanone
18. 3-Amino-1-oxido-2-[2-(trifluoromethoxy)phenyl]pyridin-4-yl}(2,4-difluorophenyl)methanone
19. [3-Amino-2-(2-methylphenyl)pyridin-4-yl](2,4-difluorophenyl)methanone
20. [3-Amino-2-(2-methylphenyl)-1-oxidopyridin-4-yl](2,4-difluorophenyl)methanone
21. {3-Amino-2-[2-(trifluoromethyl)phenyl]pyridin-4-yl}(2,4-difluorophenyl)methanone
22. {3-Amino-1-oxido-2-[2-(trifluoromethyl)phenyl]pyridin-4-yl}(2,4-difluorophenyl)methanone
23. [3-Amino-2-(2-isopropylphenyl)pyridin-4-yl](2,4-difluorophenyl)methanone
24. [3-Amino-2-(2-isopropylphenyl)-1-oxidopyridin-4-yl](2,4-difluorophenyl)methanone
25. [3-Amino-2-(2-chlorophenyl)pyridin-4-yl](2,4-difluorophenyl)methanone
26. [3-Amino-2-(2-chlorophenyl)-1-oxidopyridin-4-yl](2,4-difluorophenyl)methanone
27. [3-Amino-2-(3-chlorophenyl)pyridin-4-yl](2,4-difluorophenyl)methanone
28. [3-Amino-2-(3-chlorophenyl)-1-oxidopyridin-4-yl](2,4-difluorophenyl)methanone
29. [3-Amino-2-(4-chlorophenyl)pyridin-4-yl](2,4-difluorophenyl)methanone
30. [3-Amino-2-(4-chlorophenyl)-1-oxidopyridin-4-yl](2,4-difluorophenyl)methanone
31. [3-Amino-2-(2,6-dichlorophenyl)pyridin-4-yl](2,4-difluorophenyl)methanone
32. [3-Amino-2-(2,6-dichlorophenyl)-1-oxidopyridin-4-yl](2,4-difluorophenyl)methanone
33. [3-Amino-2-(2,6-difluorophenyl)pyridin-4-yl] (2,4-difluorophenyl)methanone
34. [3-Amino-2-(2,6-difluorophenyl)-1-oxidopyridin-4-yl](2,4-difluorophenyl)methanone
35. [3-Amino-2-(2,6-dimethylphenyl)pyridin-4-yl](2,4-difluorophenyl)methanone
36. [3-Amino-2-(2,6-dimethylphenyl)-1-oxidopyridin-4-yl](2,4-difluorophenyl)methanone
37. [3-Amino-2-(2,3-dimethoxyphenyl)pyridin-4-yl](2,4-difluorophenyl)methanone
38. [3-Amino-2-(2,3-dimethoxyphenyl)-1-oxidopyridin-4-yl](2,4-difluorophenyl)methanone
39. [3-Amino-2-(2,4-dichlorophenyl)pyridin-4-yl](2,4-difluorophenyl)methanone
40. [3-Amino-2-(2,4-dichlorophenyl)-1-oxidopyridin-4-yl](2,4-difluorophenyl)methanone
41. [3-Amino-2-(2-chloro-4-fluorophenyl)pyridin-4-yl](2,4-difluorophenyl)methanone
42. [3-Amino-2-(2-chloro-4-fluorophenyl)-1-oxidopyridin-4-yl](2,4-difluorophenyl)methanone
43. [3-Amino-2-(2,4-difluorophenyl)pyridin-4-yl](2,4-difluorophenyl)methanone
44. [3-Amino-2-(2,4-difluorophenyl)-1-oxidopyridin-4-yl](2,4-difluorophenyl)methanone
45. [3-Amino-2-(4-chloro-2-methylphenyl)pyridin-4-yl] (2,4-difluorophenyl)methanone
46. [3-Amino-2-(4-chloro-2-methylphenyl)-1-oxidopyridin-4-yl](2,4-difluorophenyl)methanone
47. [3-Amino-2-(4-hydroxy-2-methylphenyl)pyridin-4-yl](2,4-difluorophenyl)methanone
48. [3-Amino-2-(4-hydroxy-2-methylphenyl)-1-oxidopyridin-4-yl](2,4-difluorophenyl)methanone
49. [3-Amino-2-(4-[2-(3-methylphenoxy)ethyl]morpholino)pyridin-4-yl](2,4-difluorophenyl)methanone
50. [3-Amino-2-(4-[2-(3-methylphenoxy)ethyl]morpholino)-1-oxidopyridin-4-yl](2,4-difluoro-phenyl)methanone
51. {3-Amino-2-[4-(2-methoxyethoxy)-2-methylphenyl]pyridin-4-yl}(2,4-difluorophenyl)methanone
52. {3-Amino-2-[4-(2-methoxyethoxy)-2-methylphenyl]-1-oxidopyridin-4-yl}(2,4-difluorophenyl)methanone
53. 4-[3-Amino-4-(2,4-difluorobenzoyl)pyridin-2-yl]-3-methylbenzoic acid
54. 4-[3-Amino-4-(2,4-difluorobenzoyl)-1-oxidopyridin-2-yl]-3-methylbenzoic acid
55. 4-[3-Amino-4-(2,4-difluorobenzoyl)pyridin-2-yl]-3-methyl-N-(2-morpholin-4-ylethyl)benzamide
56. 4-[3-Amino-4-(2,4-difluorobenzoyl)-1-oxidopyridin-2-yl]-3-methyl-N-(2-morpholin-4-ylethyl)benzamide
57.4-[3-Amino-4-(2,4-difluorobenzoyl)pyridin-2-yl]-3-methyl-N-(2-methoxyethyl)-3-methylbenzamide
58. 4-[3-Amino-4-(2,4-difluorobenzoyl)-1-oxidopyridin-2-yl]-3-methyl-N-(2-methoxyethyl)-3-methylbenzamide
59. 4-[3-Amino-4-(2,4-difluorobenzoyl)pyridin-2-yl]-3-methyl-N-[2-(dimethylamino)ethyl]-3-methylbenzamide
60. 4-[3-Amino-4-(2,4-difluorobenzoyl)-1-oxidopyridin-2-yl]-3-methyl-N-[2-(dimethylamino)ethyl]-3-methylbenzamide
61. [3-Amino-2-(2,6-difluoro-4-methoxyphenyl)pyridin-4-yl](2,4-difluorophenyl)methanone
62. [3-Amino-2-(2,6-difluoro-4-methoxyphenyl)-1-oxidopyridin-4-yl](2,4-difluorophenyl)methanone
63. (3-Amino-3′-fluoro-2,4′-bipyridin-4-yl)(2,4-difluorophenyl)methanone
64. [3-Amino-2-(3-fluoropyridin-4-yl)-1-oxidopyridin-4-yl](2,4-difluorophenyl)methanone
65. (3-Amino-2,3′-bipyridin-4-yl)(2,4-difluorophenyl)methanone
66. (3-Amino-1-oxido-2-pyridin-3-ylpyridin-4-yl)(2,4-difluorophenyl)methanone
67. [3-Amino-2-(2-thienyl)pyridin-4-yl](2,4-difluorophenyl)methanone
68. [3-Amino-1-oxido-2-(2-thienyl)pyridin-4-yl](2,4-difluorophenyl)methanone
69. [3-Amino-2-(4-methyl-3-thienyl)pyridin-4-yl](2,4-difluorophenyl)methanone
70. [3-Amino-2-(4-methyl-3-thienyl)-1-oxidopyridin-4-yl](2,4-difluorophenyl)methanone
71. (3-Amino-2-cyclohexylpyridin-4-yl)(2,4-difluorophenyl)methanone
72. (3-Amino-2-cyclohexyl-1-oxidopyridin-4-yl)(2,4-difluorophenyl)methanone
73. [3-Amino-2-(1-naphthyl)pyridin-4-yl](2,4-difluorophenyl)methanone
74. [3-Amino-2-(1-naphthyl)-1-oxidopyridin-4-yl](2,4-difluorophenyl)methanone
75. [3-Amino-2-(2-ethoxy-1-naphthyl)pyridin-4-yl](2,4-difluorophenyl)methanone
76. [3-Amino-2-(2-ethoxy-1-naphthyl)-1-oxidopyridin-4-yl](2,4-difluorophenyl)methanone
77. [3-Amino-2-(1-benzothien-3-yl)pyridin-4-yl](2,4-difluorophenyl)methanone
78. [3-Amino-2-(1-benzothien-3-yl)-1-oxidopyridin-4-yl](2,4-difluorophenyl)methanone
79. [3-Amino-2-(1,3-benzodioxol-4-yl)pyridin-4-yl](2,4-difluorophenyl)methanone
80. [3-Amino-2-(1,3-benzodioxol-4-yl)-1-oxidopyridin-4-yl](2,4-difluorophenyl)methanone
81. [3-Amino-2-(2-methylphenyl)pyridin-4-yl](2-chlorophenyl)methanone
82. [3-Amino-2-(2-methylphenyl)-1-oxidopyridin-4-yl](2-chlorophenyl)methanone
83. [3-Amino-2-(2-methoxyphenyl)pyridin-4-yl] (2-chlorophenyl)methanone
84. [3-Amino-2-(2-methoxyphenyl)-1-oxidopyridin-4-yl](2-chlorophenyl)methanone
85. [3-Amino-2-(2-chlorophenyl)pyridin-4-yl](2-chlorophenyl)methanone
86. [3-Amino-2-(2-chlorophenyl)-1-oxidopyridin-4-yl](2-chlorophenyl)methanone
87. [3-Amino-2-(2,6-difluorophenyl)pyridin-4-yl](2-chlorophenyl)methanone
88. [3-Amino-2-(2,6-difluorophenyl)-1-oxidopyridin-4-yl](2-chlorophenyl)methanone
89. [3-Amino-2-(1,3-benzodioxol-4-yl)pyridin-4-yl](2-chlorophenyl)methanone
90. [3-Amino-2-(1,3-benzodioxol-4-yl)-1-oxidopyridin-4-yl](2-chlorophenyl)methanone
91. [3-Amino-2-(2-methylphenyl)pyridin-4-yl](3-methylphenyl)methanone
92. [3-Amino-2-(2-methylphenyl)-1-oxidopyridin-4-yl](3-methylphenyl)methanone
93. [3-Amino-2-(2-methoxyphenyl)pyridin-4-yl](3-methylphenyl)methanone
94. [3-Amino-2-(2-methoxyphenyl)-1-oxidopyridin-4-yl](3-methylphenyl)methanone
95. [3-Amino-2-(2-chlorophenyl)pyridin-4-yl](3-methylphenyl)methanone
96. [3-Amino-2-(2-chlorophenyl)-1-oxidopyridin-4-yl] (3-methylphenyl)methanone
97. [3-Amino-2-(2,6-difluorophenyl)pyridin-4-yl](3-methylphenyl)methanone
98. [3-Amino-2-(2,6-difluorophenyl)-1-oxidopyridin-4-yl] (3-methyl phenyl)methanone
99. [3-Amino-2-(1,3-benzodioxol-4-yl)pyridin-4-yl](3-methylphenyl)methanone
100. [3-Amino-2-(1,3-benzodioxol-4-yl)-1-oxidopyridin-4-yl](3-methylphenyl)methanone
101. [3-Amino-2-(2-methylphenyl)pyridin-4-yl](3-fluorophenyl)methanone
102. [3-Amino-2-(2-methylphenyl)-1-oxidopyridin-4-yl](3-fluorophenyl)methanone
103. [3-Amino-2-(2-methoxyphenyl)pyridin-4-yl](3-fluorophenyl)methanone
104. [3-Amino-2-(2-methoxyphenyl)-1-oxidopyridin-4-yl](3-fluorophenyl)methanone
105. [3-Amino-2-(2-chlorophenyl)pyridin-4-yl] (3-fluorophenyl)methanone
106. [3-Amino-2-(2-chlorophenyl)-1-oxidopyridin-4-yl] (3-fluorophenyl)methanone
107. [3-Amino-2-(2,6-difluorophenyl)pyridin-4-yl](3-fluorophenyl)methanone
108. [3-Amino-2-(2,6-difluorophenyl)-1-oxidopyridin-4-yl](3-fluorophenyl)methanone
109. [3-Amino-2-(1,3-benzodioxol-4-yl)pyridin-4-yl](3-fluorophenyl)methanone
110. [3-Amino-2-(1,3-benzodioxol-4-yl)-1-oxidopyridin-4-yl](3-fluorophenyl)methanone
111. [3-Amino-2-(2,6-dimethoxyphenyl)pyridin-4-yl](2,4-difluorophenyl)methanone
112. [3-Amino-2-(2,6-dimethoxyphenyl)-1-oxidopyridin-4-yl](2,4-difluorophenyl)methanone
113. [3-Amino-2-(2-fluorophenyl)pyridin-4-yl](2,4-difluorophenyl)methanone
114. [3-Amino-2-(2-fluorophenyl)-1-oxidopyridin-4-yl](2,4-difluorophenyl)methanone
115. [3-Amino-2-(2,6-dichlorophenyl)pyridin-4-yl](2-chlorophenyl)methanone
116. [3-Amino-2-(2,6-dichlorophenyl)-1-oxidopyridin-4-yl](2-chlorophenyl)methanone
117. [3-Amino-2-(2,6-difluorophenyl)pyridin-4-yl](2-methoxyphenyl)methanone
118. [3-Amino-2-(2,6-difluorophenyl)-1-oxidopyridin-4-yl](2-methoxyphenyl)methanone
119. [3-Amino-2-(2,6-dimethylphenyl)pyridin-4-yl](2-methoxyphenyl)methanone
120. [3-Amino-2-(2,6-dimethylphenyl)-1-oxidopyridin-4-yl](2-methoxyphenyl)methanone
121. [3-Amino-2-(2-chlorophenyl)pyridin-4-yl](2-methoxyphenyl)methanone
122. [3-Amino-2-(2-chlorophenyl)-1-oxidopyridin-4-yl](2-methoxyphenyl)methanone
123. [3-Amino-2-(2-methoxyphenyl)pyridin-4-yl](2-methoxyphenyl)methanone
124. [3-Amino-2-(2-methoxyphenyl)-1-oxidopyridin-4-yl](2-methoxyphenyl)methanone
125. [3-Amino-2-(2,6-difluorophenyl)pyridin-4-yl](2-chloro-4-fluorophenyl)methanone
126. [3-Amino-2-(2,6-difluorophenyl)-1-oxidopyridin-4-yl](2-chloro-4-fluorophenyl)methanone
127. [3-Amino-2-(2,6-dichlorophenyl)pyridin-4-yl](2-chloro-4-fluorophenyl)methanone
128. [3-Amino-2-(2,6-dichlorophenyl)-1-oxidopyridin-4-yl](2-chloro-4-fluorophenyl)methanone
129. [3-Amino-2-(2-chlorophenyl)pyridin-4-yl](2-chloro-4-fluorophenyl)methanone
130. [3-Amino-2-(2-chlorophenyl)-1-oxidopyridin-4-yl](2-chloro-4-fluorophenyl)methanone

Of outstanding interest are:
[3-Amino-2-(2-methoxyphenyl)-1-oxidopyridin-4-yl](2,4-difluorophenyl)methanone
[3-Amino-2-(2-methylphenyl)-1-oxidopyridin-4-yl](2,4-difluorophenyl)methanone
{3-Amino-1-oxido-2-[2-(trifluoromethyl)phenyl]pyridin-4-yl}(2,4-difluorophenyl)methanone
[3-Amino-2-(2-chlorophenyl)pyridin-4-yl](2,4-difluorophenyl)methanone
[3-Amino-2-(2-chlorophenyl)-1-oxidopyridin-4-yl](2,4-difluorophenyl)methanone
[3-Amino-2-(2,6-dichlorophenyl)-1-oxidopyridin-4-yl](2,4-difluorophenyl)methanone
[3-Amino-2-(2,6-difluorophenyl)pyridin-4-yl](2,4-difluorophenyl)methanone
[3-Amino-2-(2,6-difluorophenyl)-1-oxidopyridin-4-yl](2,4-difluorophenyl)methanone
[3-Amino-2-(2,6-dimethylphenyl)-1-oxidopyridin-4-yl](2,4-difluorophenyl)methanone
[3-Amino-2-(4-[2-(3-methylphenoxy)ethyl]morpholino)pyridin-4-yl](2,4-difluorophenyl)methanone
[3-Amino-2-(4-[2-(3-methylphenoxy)ethyl]morpholino)-1-oxidopyridin-4-yl](2,4-difluorophenyl)methanone
{3-Amino-2-[4-(2-methoxyethoxy)-2-methylphenyl]pyridin-4-yl}(2,4-difluorophenyl)methanone
{3-Amino-2-[4-(2-methoxyethoxy)-2-methylphenyl]-1-oxidopyridin-4-yl}(2,4-difluorophenyl)methanone
4-[3-Amino-4-(2,4-difluorobenzoyl)pyridin-2-yl]-3-methyl-N-(2-morpholin-4-ylethyl)benzamide
4-[3-Amino-4-(2,4-difluorobenzoyl)-1-oxidopyridin-2-yl]-3-methyl-N-(2-morpholin-4-ylethyl)benzamide
4-[3-Amino-4-(2,4-difluorobenzoyl)pyridin-2-yl]-3-methyl-N-(2-methoxyethyl)-3-methylbenzamide
4-[3-Amino-4-(2,4-difluorobenzoyl)-1-oxidopyridin-2-yl]-3-methyl-N-(2-methoxy-ethyl)-3-methylbenzamide
4-[3-Amino-4-(2,4-difluorobenzoyl)pyridin-2-yl]-3-methyl-N-[2-(dimethylamino)ethyl]-3-methylbenzamide
4-[3-Amino-4-(2,4-difluorobenzoyl)-1-oxidopyridin-2-yl]-3-methyl-N-[2-(dimethylamino)ethyl]-3-methylbenzamide
[3-Amino-2-(2,6-difluoro-4-methoxyphenyl)-1-oxidopyridin-4-yl](2,4-difluorophenyl)methanone
(3-Amino-2-cyclohexyl-1-oxidopyridin-4-yl)(2,4-difluorophenyl)methanone
[3-Amino-2-(1,3-benzodioxol-4-yl)pyridin-4-yl](2,4-difluorophenyl)methanone
[3-Amino-2-(1,3-benzodioxol-4-yl)-1-oxidopyridin-4-yl] (2,4-difluorophenyl)-methanone
[3-Amino-2-(2-methylphenyl)-1-oxidopyridin-4-yl](2-chlorophenyl)methanone
[3-Amino-2-(2-methoxyphenyl)-1-oxidopyridin-4-yl](2-chlorophenyl)methanone
[3-Amino-2-(2-chlorophenyl)-1-oxidopyridin-4-yl](2-chlorophenyl)methanone
[3-Amino-2-(2,6-difluorophenyl)-1-oxidopyridin-4-yl](2-chlorophenyl)methanone
[3-Amino-2-(1,3-benzodioxol-4-yl)-1-oxidopyridin-4-yl](2-chlorophenyl)methanone
[3-Amino-2-(2-methylphenyl)-1-oxidopyridin-4-yl](3-methylphenyl)methanone
[3-Amino-2-(2-methoxyphenyl)-1-oxidopyridin-4-yl](3-methylphenyl)methanone
[3-Amino-2-(2-chlorophenyl)-1-oxidopyridin-4-yl](3-methylphenyl)methanone
[3-Amino-2-(2,6-difluorophenyl)-1-oxidopyridin-4-yl](3-methylphenyl)methanone
[3-Amino-2-(1,3-benzodioxol-4-yl)-1-oxidopyridin-4-yl](3-methylphenyl)methanone
[3-Amino-2-(2,6-dimethoxyphenyl)-1-oxidopyridin-4-yl](2,4-difluorophenyl)-methanone
[3-Amino-2-(2,6-difluorophenyl)-1-oxidopyridin-4-yl](2-methoxyphenyl)methanone
[3-Amino-2-(2,6-dichlorophenyl)pyridin-4-yl](2-chloro-4-fluorophenyl)methanone

According to a further feature of the present invention, compounds of general formula (I) are prepared following the synthetic scheme illustrated in FIG. 1.

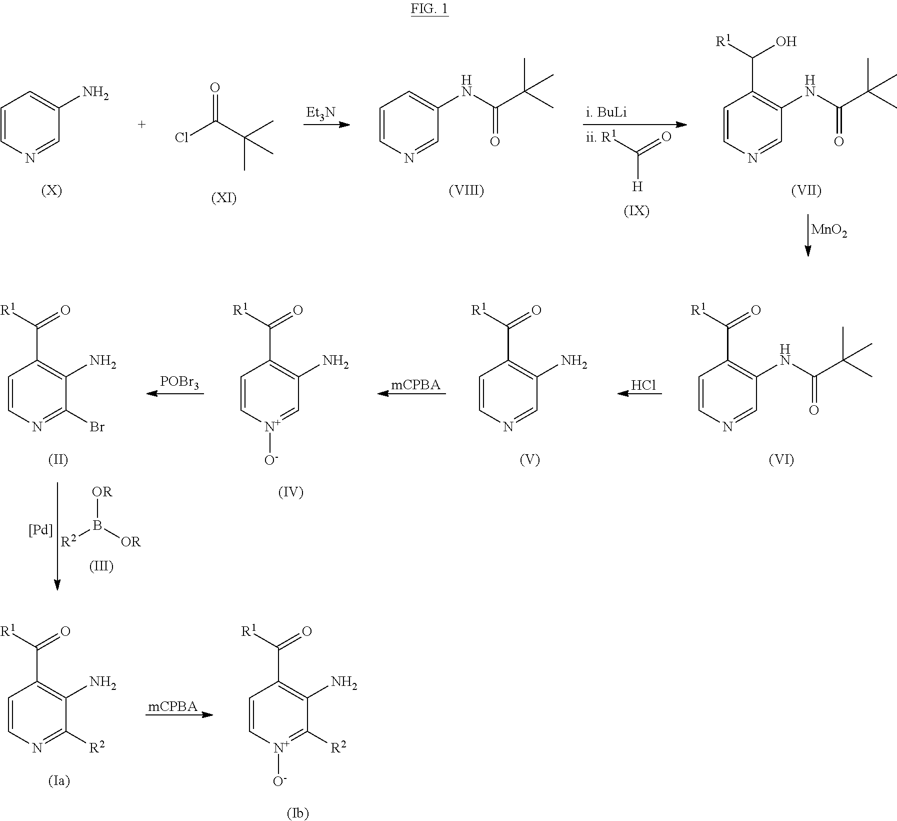

Reaction of 3-aminopyridine (X) with an acyl chloride (XI) such as pivaloyl chloride in the presence of a base such as Et₃N, or diisopropylethylamine, using an halogenated solvent such as CH₂Cl₂ or an ether solvent such as dioxane at a temperature from 0° C. to 110° C. yields compound (VIII).

Compounds of formula (VII) can be obtained by lithiation of the compounds of formula (VIII) with a solution of BuLi in hexanes, possibly in the presence of a cosolvent such as N,N,N′,N′-tetramethylethane-1,2-diamine and subsequent addition of the corresponding aldehydes of formula (IX) at a temperature from −78° C. to r.t.

Oxidation of the alcohol compound (VII) with an oxidizing agent such as manganese dioxide, Dess-Martin periodinane, tetrapropyl-ammonium perruthenate or pyridinium chlorochromate, preferably with manganese dioxide in an halogenated solvent such as CHCl₃ at a temperature from r.t. to the boiling point of the solvent yields the compounds of formula (VI).

Subsequent hydrolysis of the pivalamide group in compounds of formula (VI) in acidic conditions such as treatment with HCl 5N using a solvent miscible with water like ethanol at a temperature from 100° C. to 150° C. yields the aminopyridine of formula (V).

The pyridine N-oxide of formula (IV) may be obtained by oxidation of the aminopyridine of formula (V) with an oxidizing agent such as Oxone®, magnesium monoperoxyphthalate hexahydrate, hydrogen peroxide or meta-chloroperbenzoic acid, preferably with meta-chloroperbenzoic in an halogenated solvent like CH₂Cl₂ and a temperature from 0° C. to the boiling point of the solvent.

The intermediate of formula (II) may be obtained by reacting the pyridine N-oxide of formula (IV) with phosphorus oxybromide neat or in an halogenated solvent like CH₂Cl₂ at a temperature from 60° C. to 140° C.

The compounds of formula (Ia) may be obtained by coupling a bromoderivative of formula (II) with the corresponding boronic acids or boronates of formula (III) using Suzuki reactions (Miyaura, N.; Suzuki, A. Chem. Rev. 1995, 95, 2457). These reactions may be catalized by a palladium catalyst like [1,1′-bis(diphenylphosphino)-ferrocene]dichloropalladium (II) complex with dichloromethane (1:1), tetrakis(triphenylphosphine)palladium(0), bis(triphenylphosphine)palladium(II) chloride or tris(dibenzylideneacetone)dipalladium(0) in an aprotic organic solvent such as dioxane, toluene, DMF or DME and in the presence of a base such as cesium carbonate, sodium carbonate or potassium phosphate at a temperature from 80° C. to 140° C.

The nitrogen atom of the pyridine ring in the compounds of formula (Ia) may, when the groups R¹ and R² are aromatic or heteroaromatic groups substituted with alkyl groups, alkoxy groups, hydroxy groups, halogens, carboxylic acid groups, amide groups not containing aminoalkyl chains, be oxidised with an oxidizing agent such as Oxone®, magnesium monoperoxyphthalate hexahydrate, hydrogen peroxide or meta-chloroperbenzoic acid, preferably with meta-chloroperbenzoic in an halogenated solvent like CH₂Cl₂, at a temperature from 0° C. to the boiling point of the solvent to yield the pyridine oxides of formula (Ib).

In the particular case where R² is an aromatic or heteroaromatic group o,o′-disubstituted by alkyl groups, alkoxy groups or halogens the bromoderivative of formula (II) may be coupled with the corresponding boronic acid or boronate by a Suzuki reaction (Miyaura, N.; Suzuki, A. Chem. Rev. 1995, 95, 2457) using a palladium catalyst such as tris(dibenzylideneacetone)dipalladium(0) in the presence of a ligand such as 2-(dicyclohexylphosphino)-2′,6′-dimethoxy-1-1′-biphenyl (S—PHOS) and a base like potassium phosphate, and in a solvent such as toluene at a temperature from 80° C. to 140° C. to yield the compound of formula (Ia).

In the particular case where R² is an o,o′-difluoro substituted phenyl ring the bromoderivative (II) may be coupled with the corresponding 1,3-difluorobenzene by a Negishi reaction (Negishi, E.-I.; Baba, S. J. Chem. Soc., Chem. Commun. 1976, 596) to yield the compound (Ia). In this reaction, the first step is the lithiation of 1,3-difluorobenzene by treatment with a base such as BuLi at −78° C. using THF as solvent, afterwards a transmetalation step is carried out by treatment of the corresponding organolithium derivative with zinc dichloride at −50° C. and finally, the resulting organozinc is coupled with the bromoderivative of formula (II) using a palladium catalyst such as tetrakis(triphenylphosphine)palladium(0), bis(triphenylphosphine)-palladium(II) chloride or tris(dibenzylideneacetone)dipalladium(0) at a temperature between r.t. and the boiling point of the solvent.

In the particular case where R² is a carbocycle or heterocycle optionally substituted by alkyl groups or alkoxy groups the bromoderivative of formula (II) may be coupled with the corresponding organozinc through a Negishi coupling, using [1,1′-bis(diphenylphosphino)ferrocene]dichloropalladium (II) complex with dichloromethane (1:1) and copper (I) iodide as catalysts and THF as solvent at a temperature of 120° C. in a sealed vessel under microwave irradiation.

In the particular case where R² is a phenyl ring substituted among other groups with a morpholinoethoxy group the synthetic scheme of FIG. 2 shown below may be used.

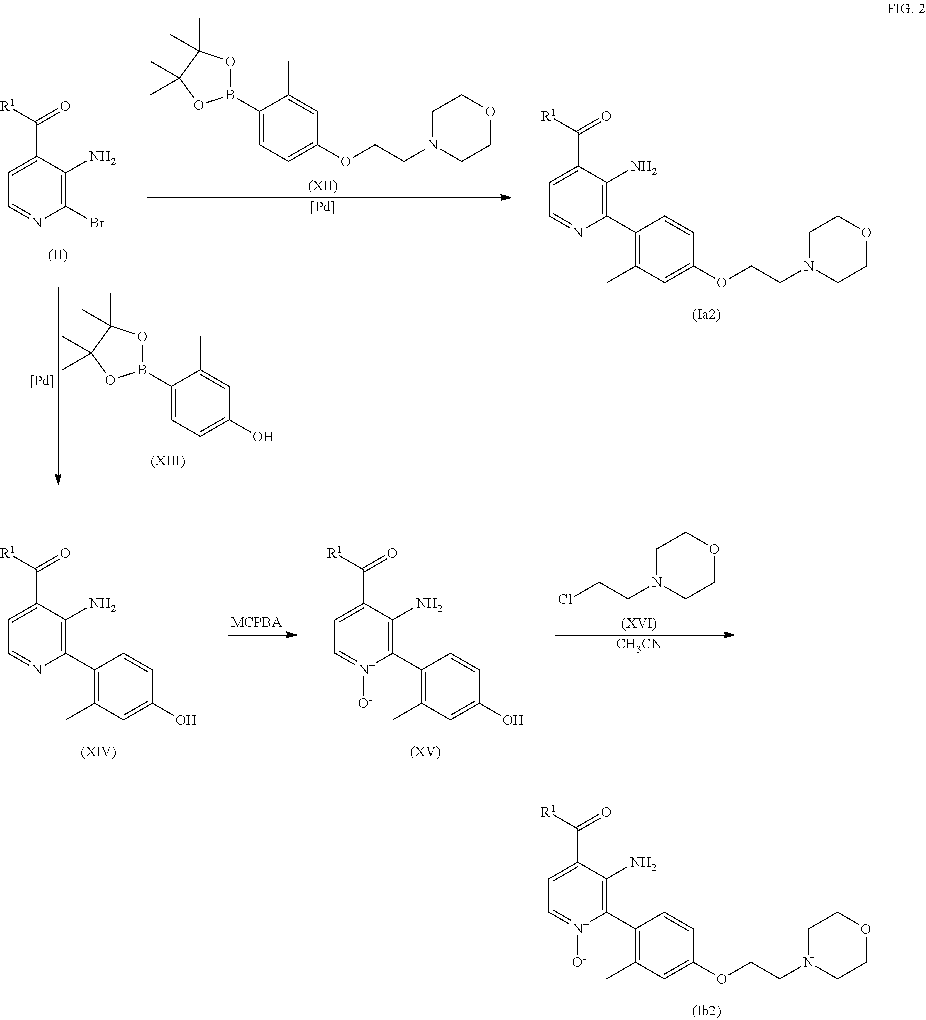

In one alternative the bromo derivative of formula (II) may be reacted with a boronate of formula (XII) using a palladium catalyst such as [1,1′-bis(diphenylphosphino)ferrocene]dichloropalladium (II) complex with dichloromethane (1:1), tetrakis(triphenylphosphine)-palladium(0), bis(triphenylphosphine)palladium(II) chloride or tris(dibenzylideneacetone)-dipalladium(0) in an aprotic organic solvent like dioxane, toluene, DMF or DME and in the presence of a base such as cesium carbonate, sodium carbonate or potassium phosphate at a temperature from 80° C. to 140° C. to obtain product (Ia2).

In another alternative the bromo derivative of formula (II) may be reacted with a boronate of formula (XIII) in the presence of a palladium catalyst such as [1,1′-bis(diphenylphosphino)ferrocene]dichloropalladium (II) complex with dichloromethane (1:1), tetrakis(triphenylphosphine)-palladium(0), bis(triphenylphosphine)palladium(II) chloride or tris(dibenzylideneacetone)dipalladium(0) in an aprotic organic solvent such as dioxane, toluene, DMF or DME and in the presence of a base such as cesium carbonate, sodium carbonate or potassium phosphate at a temperature from 80° C. to 140° C. to yield product (XIV). This product may be oxidized with an oxidizing agent such as Oxone®, magnesium monoperoxyphthalate hexahydrate, hydrogen peroxide or meta-chloroperbenzoic acid, preferably with meta-chloroperbenzoic in an halogenated solvent such as CH₂Cl₂ and a temperature from 0° C. to the boiling point of the solvent to the corresponding N-oxide (XV). Finally, this N-oxide (XV) is reacted with the morpholino derivative of formula (XVI) in the presence of a base such as potassium carbonate in an aprotic organic solvent such as CH₃CN to yield the desired compound (Ib2).

In the particular case where R² is a phenyl ring substituted among other groups with a methoxyethoxy group, the synthetic scheme of FIG. 3 shown below may be used.

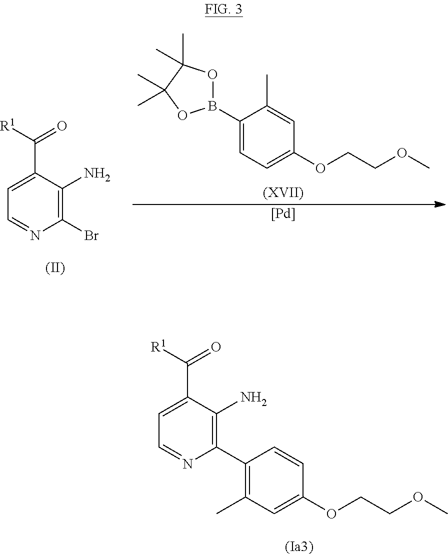

The bromoderivative of formula (II) is coupled to boronate of formula (XVII) using a palladium catalyst such as [1,1′-bis(diphenylphosphino)ferrocene]dichloropalladium (II) complex with dichloromethane (1:1), tetrakis(triphenylphosphine)-palladium(0), bis(triphenylphosphine)palladium(II) chloride or tris(dibenzylideneacetone)dipalladium(0) in an aprotic organic solvent such as dioxane, toluene, DMF or DME and in the presence of a base such as cesium carbonate, sodium carbonate or potassium phosphate at a temperature from 80° C. to 140° C. to yield the compound (Ia3).

In the particular case where R² is a phenyl ring substituted among other groups with an amide group, synthetic scheme of FIG. 4 may be used.

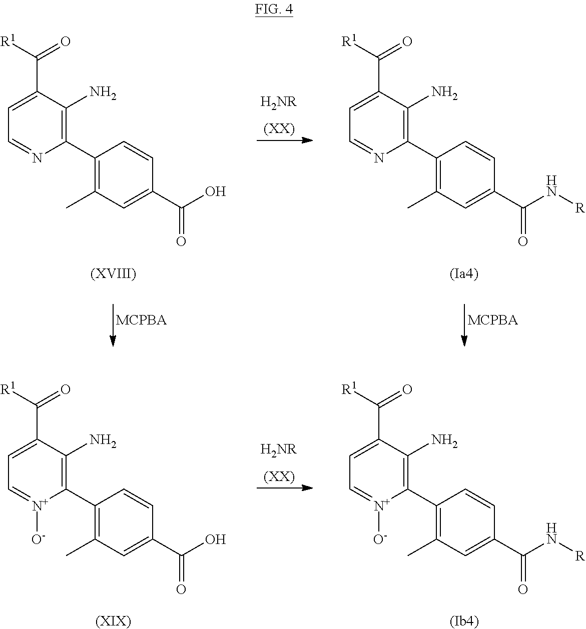

The acid compound of formula (XVIII) is reacted with the corresponding amine (XX) in the presence of an amidation reagent such 2-benzotriazol-1-yl-N,N,N′,N′-tetramethyluronio hexafluorphosphate (HBTU), 2-benzotriazol-1-yl-N,N,N′,N′-tetramethyluronio tetrafluoroborate (TBTU) or 1-(3-dimethylaminopropyl)-3-ethylcarbodiimide hydrochloride (EDC) and an organic base such as diisopropylethylamine in an aprotic organic solvent such as DMF or CH₃CN at r.t. to yield the compound of formula (Ia4)

For the case of amides not containing aminoalkyl groups, product (Ia4) may be treated with an oxidizing reagent such as Oxone®, magnesium monoperoxyphthalate hexahydrate, hydrogen peroxide or meta-chloroperbenzoic acid, preferably with meta-chloroperbenzoic acid in an halogenated solvent such as CH₂Cl₂ to obtain the corresponding N-oxides (Ib4).

When the amides do contain aminoalkyl groups, the acid compound of formula (XVIII) is oxidized first to the corresponding N-oxide (XIX) by treatment with an oxidizing reagent such as Oxone®, magnesium monoperoxyphthalate hexahydrate, hydrogen peroxide or meta-chloroperbenzoic acid, preferably with meta-chloroperbenzoic acid in an halogenated solvent such as CH₂Cl₂ and afterwards, the amidation reaction is carried out by treatment of intermediate (XIX) with the corresponding amine (XX) in the presence of an amidation reagent such as 2-benzotriazol-1-yl-N,N,N′,N′-tetramethyluronio hexafluorphosphate (HBTU), 2-benzotriazol-1-yl-N,N,N′,N′-tetramethyluronio tetrafluoroborate (TBTU) or 1-(3-dimethylaminopropyl)-3-ethylcarbodiimide hydrochloride (EDC) and an organic base such as diisopropylethylamine in an aprotic organic solvent such as DMF or CH₃CN at r.t to yield compounds (Ib4).

When the groups R¹ and R² are susceptible to chemical reaction under the conditions of the hereinbefore described processes or are incompatible with said processes, alternative processes can be readily carried out utilising organic synthetic chemistry methods to, for example, protect functional groups and finally eliminate protecting groups.

The pyridin-3-amine derivatives of formula (I) can be converted by methods known per se into pharmaceutically acceptable salts or N-oxides. Preferred salts are acid addition salts obtainable by treatment with organic or inorganic acids such as fumaric, tartaric, succinic or hydrochloric acid. Also pyridin-3-amine derivatives of formula (I) in which there is the presence of an acidic group may be converted into pharmacologically acceptable salts by reaction with an alkali metal hydroxide or an organic base such as sodium or potassium hydroxide. The acid or alkali addition salts so formed may be interchanged with suitable pharmaceutically acceptable counter ions using processes known per se.

### Biological Testing

### Inhibition Assay

Enzymatic activity assay was performed in 96-well microtiter plates (Corning, catalog number # 3686) using a total volume of 50 μl of an assay buffer composed of 50 mM HEPES pH 7.5, 10 mM MgCl₂, 1.75 mM Na₃VO₄.

Various concentrations of the test compound or vehicle controls were pre-incubated for one hour with 0.055 μg/ml of the human p38alfa (SAPKa) enzyme (obtained from University of Dundee). The reaction started by addition of biotinylated ATF2 substrate and ATP in concentrations around their Km values (final concentration 0.62 μM and 60 μM respectively) and took place for one hour at 25° C. Addition of the detection reagents, streptavidin-XL665 and anti-phosphoresidue antibody coupled to Europium cryptate, caused the juxtaposition of the cryptate and the XL665 fluorophore, resulting in fluorescence energy transfer (FRET). The FRET intensity depends on the amount of bounded cryptate antibody, which is proportional to the extent of substrate phosphorylation. FRET intensity was measured using Victor 2V spectrofluorometer. Data were analyzed by non-linear regression (Hill equation) to generate a dose-response curve. The calculated IC₅₀ value is the concentration of the test compound that caused a 50% decrease in the maximal FRET intensity.

### Functional Assay

The activity of compounds in inhibiting TNFα production was measured in a cellular assay using the human monocytic cell line THP-1. For this purpose, 2×10⁵ cells/well were plated in tissue-culture treated round-bottom 96-well plates in RPMI (containing 10% FCS, L-Gln 2 mM, Hepes buffer 10 mM, sodium pyruvate 1 mM, glucose 4.5 gr/L, HNaCO₃ 1.5 g/L and beta-mercaptoethanol 50 μM), together with compounds at the desired test concentration and LPS (Sigma, L2630) at a final 10 μg/ml concentration. Compounds were resuspended in 100% DMSO at a concentration of 1 mM and titrated thereof in 10× dilutions in medium. Controls included stimulated cells alone and stimulated cells treated with the highest concentration of compound vehicle (1% DMSO). Cells were incubated for 5 h at 37° C. in a 5% CO₂ atmosphere. Cell supernatant was recovered by centrifugation and diluted 5-fold prior to testing in a standard human TNFα ELISA (RnD systems).

Data were analyzed by non-linear regression (Hill equation) to generate a dose-response curve. The calculated IC₅₀ value is the concentration of the test compound that caused a 50% decrease in the maximal TNFα production.

Table 1 shows the activities in THP-1 assay of some compounds of the present invention.

TABLE 1

| Example | p38α IC₅₀ (nM) | THP-1 TNFα IC₅₀ (nM) |
|---|---|---|
| 20 | 76 | 39 |
| 25 | 251 | 130 |
| 26 | 45 | 76 |
| 32 | 25 | 19.4 |
| 34 | 15 | 36 |
| 36 | 6 | 33 |
| 47 | 277 | 402 |
| 48 | 6 | 84 |
| 79 | 103 | 53 |
| 82 | 31 | 118 |
| 92 | 34 | 54 |
| 98 | 20 | 48 |
| 109 | 359 | 349 |
| 118 | 86 | 140 |
| 120 | 146 | 98 |

It can be seen from Table 1 that the compounds of formula (I) are potent inhibitors of the p38 mitogen-activated protein kinase. Preferred pyridin-3-amine derivatives of the invention possess a IC₅₀ value of binding to p38α of less than 10 μM, preferably less than 1 μM, more preferably less than 100 nM and most preferably less than 10 nM.

It can also be seen that the compounds of formula (I) are good inhibitors of TNFα production. Preferred pyridin-3-amine derivatives of the invention possess a IC₅₀ value for inhibiting TNFα production of less than 100 μM, preferably less than 10 μM, more preferably less than 1 μM and most preferably less than 100 nM.

The pyridin-3-amine derivatives of the invention are useful in the treatment or prevention of diseases known to be susceptible to improvement by inhibition of the p38 mitogen-activated protein kinase. Such diseases are, for example rheumatoid arthritis, ischemia-reperfusion injury, cerebral focal ischemia, acute coronary syndrome, COPD, Crohn's disease, irritable bowel syndrome, adult respiratory distress syndrome, osteoporosis Alzheimer's disease, rheumatoid spondylitis, psoriasis, atherosclerosis, osteoarthritis or multiple myeloma.

Accordingly, the pyridin-3-amine derivatives of the invention and pharmaceutically acceptable salts thereof, and pharmaceutical compositions comprising such compound and/or salts thereof, may be used in a method of treatment of disorders of the human body which comprises administering to a subject requiring such treatment an effective amount of pyridin-3-amine derivative of the invention or a pharmaceutically acceptable salt thereof.

When the pyridin-3-amine derivatives of the invention are used for the treatment of respiratory diseases such as asthma, chronic obstructive pulmonary disorder, pulmonary fibrosis or emphysema it may be advantageous to use them in combination with other active compounds known to be useful in the treatment of respiratory diseases such as (1) antagonists of M3 muscarinic receptors, (2) β2-agonists, (3) PDE4 inhibitors, (4) cortiocosteroids, (5) leukotriene D4 antagonists, (6) inhibitors of egfr-kinase, (7) antagonists of the A2B adenosine receptor, (8) NK1 receptor agonists, (9) CRTh2 antagonists, (10) syk kinase inhibitors, (11) CCR3 antagonists and (12) VLA-4 antagonists.

Thus, the present invention also provides pharmaceutical compositions comprising a pyridin-3-amine derivative of the invention and another active compound selected from the groups consisting of (1) antagonists of M3 muscarinic receptors, (2) β2-agonists, (3) PDE 4 inhibitors, (4) cortiocosteroids, (5) leukotriene D4 antagonists, (6) inhibitors of egfr-kinase, (7) antagonists of the A2B adenosine receptor, (8) NK1 receptor agonists, (9) CRTh2 antagonists, (10) syk kinase inhibitors, (11) CCR3 antagonists and (12) VLA-4 antagonists.

The present invention also provides pharmaceutical compositions which comprise, as an active ingredient, at least a pyridin-3-amine derivative of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application. Preferably the compositions are made up in a form suitable for oral, topical, nasal, rectal, percutaneous or injectable administration.

The pharmaceutically acceptable excipients which are admixed with the active compound or salts of such compound, to form the compositions of this invention are well-known per se and the actual excipients used depend inter alia on the intended method of administering the compositions.

Compositions of this invention are preferably adapted for injectable and per os administration. In this case, the compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Effective doses are normally in the range of 2-2000 mg of active ingredient per day. Daily dosage may be administered in one or more treatments, preferably from 1 to 4 treatments, per day.

The syntheses of the compounds of the invention and of the intermediates for use therein are illustrated by the following Examples (1 to 130) including Preparation Examples (Preparations 1-12) which do not limit the scope of the invention in any way.

¹H Nuclear Magnetic Resonance Spectra were recorded on a Varian Gemini 300 spectrometer. Melting points were recorded using a Büchi B-540 apparatus. The chromatographic separations were obtained using a Waters 2795 system equipped with a Symmetry C18 (2.1×100 mm, 3.5 mm) column. As detectors a Micromass ZMD mass spectrometer using ES ionization and a Waters 996 Diode Array detector were used. The mobile phase was formic acid (0.46 ml), ammonia (0.115 ml) and water (1000 ml) (A) and formic acid (0.4 ml), ammonia (0.1 ml), methanol (500 ml) and acetonitrile (500 ml) (B): initially from 0% to 95% of B in 20 min, and then 4 min. with 95% of B. The reequilibration time between two injections was 5 min. The flow rate was 0.4 ml/min. The injection volume was 5 μl. Diode array chromatograms were processed at 210 nm.

### PREPARATION EXAMPLES

### Preparation 1

### (3-Amino-2-bromopyridin-4-yl)(phenyl)methanone

### a) 2,2-Dimethyl-N-pyridin-3-ylpropanamide

To an ice-cooled solution of 3-aminopyridine (6 g, 63.8 mmol) and triethyl amine (9.72 mL, 70.2 mmol) in 124 mL of dichloromethane under argon, was carefully added pivaloyl chloride (7.92 mL, 64.4 mmol) in 16 mL of dichloromethane. After the addition was completed, the reaction mixture was stirred at 0° C. for 15 minutes and at room temperature for 18 hours. The mixture was washed with water, aqueous 4% sodium bicarbonate, brine, dried over sodium sulphate and the solvent removed under reduced pressure. The residue was purified by column chromatography on silica flash, using hexane/ethyl acetate (85:15) as eluents, to yield the title compound (8.5 g, 75%) as a white solid.

### b) N-{4-[Hydroxy(phenyl)methyl]pyridin-3-yl}-2,2-dimethylpropanamide

nBuLi (2.5M in hexanes, 11.2 mL, 28 mmol) was dropwise added to a solution of the title compound of Preparation 1a (2 g, 11.2 mmol) in dry tetrahydrofuran (28 mL) at −78° C. under argon and the resulting mixture was stirred at that temperature for 15 minutes and at 0° C. for 3 hours. Then, the reaction mixture was cooled down to −78° C. and benzaldehyde (1.72 mL, 16.8 mmol) in 2.8 mL of tetrahydrofuran was carefully added. After 15 minutes, the cooling bath was removed and the mixture stirred overnight at room temperature. Subsequently, water was added to the flask and it was extracted with ethyl acetate (3×50 ml), the organic solution was washed with brine, dried over sodium sulphate and the solvent removed under reduced pressure. The residue was purified by column chromatography on silica flash, using hexane/ethyl acetate (1:2 to ethyl acetate) as eluents, to yield the title compound (2.16 g, 54%) as a solid.

### c) N-(4-Benzoylpyridin-3-yl)-2,2-dimethylpropanamide

The title compound of Preparation 1b (2.16 g, 7.6 mmol) was dissolved in chloroform (65 mL) and activated manganese (IV) oxide (6.61 g, 76 mmol) was portionwise added during 1 hour. The suspension was stirred at room temperature for 16 hours. The mixture was filtered through Celite®, washed with more chloroform and the solvent evaporated to afford the title compound (2.18 g, 99%) as a solid.

### d) (3-Aminopyridin-4-yl)(phenyl)methanone

A solution of the title compound of Preparation 1c (3 g, 10.5 mmol) in 30 mL of ethanol was treated with HCl 5N (90 mL) and heated to 98° C. for 6 hours. The reaction mixture was cooled down, poured into ice water and the pH adjusted to 9-10 with concentrated aqueous ammonia. The solution was extracted with ethyl acetate (2×250 ml), the organic layer was washed with brine, dried over sodium sulphate and the solvent removed under reduced pressure. The residue was triturated with hexane/diethyl ether (5:1) to yield the title compound (1.2 g, 60%) as a yellowish solid.

### e) (3-Amino-1-oxidopyridin-4-yl)(phenyl)methanone

To a solution of the title compound of Preparation 1d (800 mg, 4 mmol) in dichloromethane (20 ml) at 0° C. was portionwise added meta-chloroperbenzoic acid (1.2 g, 6 mmol) and the reaction mixture was stirred overnight at room temperature. Then, more dichloromethane was added (50 ml) and the solution was washed with aqueous sodium bicarbonate 4% (3×30 ml) and brine. The organic layer was dried over sodium sulphate and concentrated under reduced pressure to give a residue that was triturated in a mixture of hexane and ethyl acetate (9:1) and filtered to yield the title compound (771 mg, 90%) as a yellow solid.

### f) (3-Amino-2-bromopyridin-4-yl)(phenyl)methanone

The title compound of Preparation 1e (520 mg, 2.43 mmol) was dissolved in 15 mL of dry dichloromethane and phosphorus oxybromide (2.08 g, 7.28 mmol) portionwise added. The mixture was stirred at 60° C. for 3 hours. The reaction was cooled down, poured into ice water and the pH adjusted to 10-11 with concentrated aqueous ammonia. The solution was extracted with ethyl acetate (2×200 mL), the organic layer was washed with brine, dried over sodium sulphate and the solvent removed under reduced pressure. The residue was purified by column chromatography on silica flash, using hexane/ethyl acetate (3:1) as eluents, to yield the title compound (390 mg, 58%) as a bright yellow solid.

H¹-NMR δ (CDCl₃): 6.40 (bs, 2H), 7.46-7.70 (m, 6H), 7.72 (d, J=6 Hz, 1H).

### Preparation 2

### (3-Amino-2-bromopyridin-4-yl)(2,4-difluorophenyl)methanone

### a) N-{4-[(2,4-Difluorophenyl)(hydroxy)methyl]pyridin-3-yl}N-2,2-dimethylpropanamide

Obtained as a yellow solid (54%) from the title compound of Preparation 1a and 2,4-difluorobenzaldehyde following the experimental procedure described in Preparation 1b.

### b) N-[4-(2,4-Difluorobenzoyl)pyridin-3-yl]-2,2-dimethylpropanamide

Obtained as a yellow solid (99%) from the title compound of Preparation 2a following the experimental procedure described in Preparation 1c.

### c) (3-Aminopyridin-4-yl)(2,4-difluorophenyl)methanone

Obtained as a bright yellow solid (75%) from the title compound of Preparation 2b following the experimental procedure described in Preparation 1d.

### d) (3-Amino-1-oxidopyridin-4-yl)(2,4-difluorophenyl)methanone

Obtained as a bright yellow solid (80%) from the title compound of Preparation 2c following the experimental procedure described in Preparation 1e.

### e) (3-Amino-2-bromopyridin-4-yl)(2,4-difluorophenyl)methanone

Obtained as a bright yellow solid (56%) from the title compound of Preparation 2d following the experimental procedure described in Preparation 1f.

H¹-NMR δ (CDCl₃): 6.75 (bs, 2H), 6.88-7.09 (m, 2H), 7.12 (dd, J=2 and 4 Hz, 1H), 7.45-7.56 (m, 1H), 7.70 (d, J=6 Hz, 1H).

### Preparation 3

### (3-Amino-2-bromopyridin-4-yl)(2-chlorophenyl)methanone

### a) N-{4-[(2-Chlorophenyl)(hydroxy)methyl]pyridin-3-yl}-2,2-dimethylpropanamide

Obtained as a solid (33%) from the title compound of Preparation 1a and 2-chlorobenzaldehyde following the experimental procedure described in Preparation 1b.

### b) N-[4-(2-Chlorobenzoyl)pyridin-3-yl]-2,2-dimethylpropanamide

Obtained as a yellow solid (97%) from the title compound of Preparation 3a following the experimental procedure described in Preparation 1c.

### c) (3-Aminopyridin-4-yl)(2-chlorophenyl)methanone

Obtained as a bright yellow solid (95%) from the title compound of Preparation 3b following the experimental procedure described in Preparation 1d.

### d) (3-Amino-1-oxidopyridin-4-yl)(2-chlorophenyl)methanone

Obtained as a bright yellow solid (88%) from the title compound of Preparation 3c following the experimental procedure described in Preparation 1e.

### e) (3-Amino-2-bromopyridin-4-yl)(2-chlorophenyl)methanone

Obtained as a bright yellow solid (57%) from the title compound of Preparation 3d following the experimental procedure described in Preparation 1f.

H¹-NMR δ (CDCl₃): 6.90 (bs, 2H), 6.98 (d, J=6 Hz, 1H), 7.29-7.49 (m, 4H), 7.64 (d, J=6 Hz, 1H).

### Preparation 4

### (3-Amino-2-bromopyridin-4-yl)(3-methylphenyl)methanone

### a) N-{4-[Hydroxy(3-methylphenyl)methyl]pyridin-3-yl}-2,2-dimethylpropanamide

nBuLi (2.5M in hexanes, 4.7 mL, 7.5 mmol) was dropwise added to a solution of the title compound of Preparation 1a (534 mg, 3 mmol) and N,N,N′,N′-tetramethylethylenediamine (TMEDA) (1.12 mL, 7.5 mmol) in diethyl ether (18 mL) at −78° C. under argon and the resulting mixture was stirred at that temperature for 15 minutes and at −10° C. for 2 hours. Then, the reaction mixture was cooled down to −78° C. and 3-methylbenzaldehyde (0.88 mL, 7.5 mmol) in 1.8 mL of dry tetrahydrofuran was carefully added. After 15 minutes, the cooling bath was removed and the mixture stirred overnight at room temperature. Subsequently, water was added to the flask and it was extracted with ethyl acetate (3×25 ml), the organic solution was washed with brine, dried over sodium sulphate and the solvent removed under reduced pressure. The residue was purified by column chromatography on silica flash, using hexane/ethyl acetate (1:4) as eluents, to yield the title compound (543 mg, 57%) as a solid.

### b) 2,2-Dimethyl-N-[4-(3-methylbenzoyl)pyridin-3-yl]propanamide

Obtained as a yellow solid (99%) from the title compound of Preparation 4a following the experimental procedure described in Preparation 1c.

### c) (3-Aminopyridin-4-yl)(3-methylphenyl)methanone

Obtained as a bright yellow solid (97%) from the title compound of Preparation 4b following the experimental procedure described in Preparation 1d.

### d) (3-Amino-1-oxidopyridin-4-yl)(3-methylphenyl)methanone

Obtained as a bright yellow solid (51%) from the title compound of Preparation 4c following the experimental procedure described in Preparation 1e.

### e) (3-Amino-2-bromopyridin-4-yl)(3-methylphenyl)methanone

Obtained as a bright yellow solid (37%) from the title compound of Preparation 4d following the experimental procedure described in Preparation 1f.

H¹-NMR δ (CDCl₃): 2.43 (s, 3H), 6.38 (bs, 2H), 7.25 (d, J=6 Hz, 1H), 7.37-7.49 (m, 4H), 7.72 (d, J=6 Hz, 1H).

### Preparation 5

### (3-Amino-2-bromopyridin-4-yl)(3-fluorophenyl)methanone

### a) N-{4-[(3-Fluorophenyl)(hydroxy)methyl]pyridin-3-yl}-2,2-dimethylpropanamide

Obtained as a solid (44%) from the title compound of Preparation 1a and 3-fluorobenzaldehyde following the experimental procedure described in Preparation 4a (recrystallized from EtOAc).

### b) N-[4-(3-Fluorobenzoyl)pyridin-3-yl]-2,2-dimethylpropanamide

Obtained as a yellow solid (92%) from the title compound of Preparation 5a following the experimental procedure described in Preparation 1c.

### c) (3-Aminopyridin-4-yl)(3-fluorophenyl)methanone

Obtained as a bright yellow solid (90%) from the title compound of Preparation 5b following the experimental procedure described in Preparation 1d.

### d) (3-Amino-1-oxidopyridin-4-yl)(3-fluorophenyl)methanone

Obtained as a bright yellow solid (64%) from the title compound of Preparation 5c following the experimental procedure described in Preparation 1e.

### e) (3-Amino-2-bromopyridin-4-yl)(3-fluorophenyl)methanone

Obtained as a bright yellow solid (31%) from the title compound of Preparation 5d following the experimental procedure described in Preparation 1f.

H¹-NMR δ (CDCl₃): 6.46 (bs, 2H), 7.24 (d, J=6 Hz, 1H), 7.29-7.55 (m, 4H), 7.73 (d, J=6 Hz, 1H).

### Preparation 6

### 3-Methyl-4-(4,4,5,5-tetramethyl-[1,3,2]dioxaborolan-2-yl)-phenol

In a Schlenk tube a mixture of 4-bromo-3-methylphenol (805 mg, 4.23 mmol), 4,4,5,5,4′,4′,5′,5′-Octamethyl-[2,2′]bi[[1,3,2]dioxaborolanyl] (1.64 g, 6.45 mmol), [1,1′-bis(diphenyl-phosphino)ferrocene]dichloropalladium(II) complex with dichloromethane (1:1) (344 mg, 0.42 mmol) and potassium acetate (2.1 g, 21 mmol) in N,N-dimethyl formamide (15 mL) was heated at 80° C. for 18 hours. The cooled mixture was diluted with ethyl acetate, washed with water, brine and dried over sodium sulphate. The solvent was removed under reduced pressure and the residue was purified by column chromatography on silica flash, using hexane/ethyl acetate (7:1) as eluents, to yield the title compound (617 mg, 63%) as a white solid.

H¹-NMR δ (CDCl₃): 1.32 (s, 12H), 2.49 (s, 3H), 6.62-6.64 (m, 2H), 7.64-7.69 (d, J=10H, 1H).

### Preparation 7

### 1,3-Benzodioxole-4-boronic acid

nBuLi (2.5M in hexanes, 2.38 mL, 5.97 mmol) was dropwise added to a solution of 4-bromo-1,3-benzodioxole (1 g, 4.97 mmol) and triisopropyl borate (1.49 mL, 6.47 mmol) in 50 mL of dry tetrahydrofuran at −78° C. under argon. The reaction was maintained at that temperature for 3 hours, then warmed up to room temperature and cooled back to 0° C. immediately. The solution was acidified to pH=2 with HCl 2N and neutralized to pH=7 with NaOH 2N, it was then extracted with ethyl acetate (3×25 ml), the organic solution was washed with brine, dried over sodium sulphate and the solvent removed under reduced pressure to yield the title compound (570 mg, 69%) as a white solid.

H¹-NMR δ (CD3OD): 5.92 (s, 2H), 6.80-6.86 (m, 3H).

### Preparation 8

### 4-{2-[3-Methyl-4-(4,4,5,5-tetramethyl-[1,3,2]dioxaborolan-2-yl)-phenoxy]-ethyl}-morpholine

### a) 4-[2-(4-Bromo-3-methylphenoxy)ethyl]morpholine

To a solution of 4-bromo-3-methylphenol (508 mg, 2.66 mmol) in 25 mL of acetonitrile were added 4-(2-chloroethyl)morpholine hydrochloride (540 mg, 2.88 mmol) and potassium carbonate (1.45 g, 10.49 mmol) and the mixture was heated to 80° C. for 3 h. The reaction was cooled down and filtered through a pad of Celite® washing with acetonitrile (10 mL). The solvent was removed under reduced pressure to give the title compound (828 mg, 99%) as a solid.

### b) 4-{2-[3-Methyl-4-(4,4,5,5-tetramethyl-[1,3,2]dioxaborolan-2-yl)-phenoxy]-ethyl}-morpholine

In a Schlenk tube a mixture of the title compound from Preparation 8a (670 mg, 2.25 mmol), 4,4,5,5,4′,4′,5′,5′-Octamethyl-[2,2′]bi[[1,3,2]dioxaborolanyl] (840 mg, 3.3 mmol), [1,1′-bis(diphenyl-phosphino)ferrocene]dichloropalladium(II) complex with dichloromethane (1:1) (172 mg, 0.21 mmol) and potassium acetate (1.06 g, 10.8 mmol) in N,N-dimethyl formamide (10 mL) was heated at 80° C. for 18 hours. The cooled mixture was diluted with ethyl acetate, washed with water, brine and dried over sodium sulphate. The solvent was removed under reduced pressure and the residue was purified by column chromatography on silica flash, using hexane/ethyl acetate (1:4) as eluents, to yield the title compound (554 mg, 71%).

H¹-NMR δ (CDCl₃): 1.32 (s, 12H), 2.51 (s, 3H), 2.56-2.60 (m, 2H), 2.80 (t, J=6 Hz, 2H), 3.71-3.76 (m, 2H), 4.12 (t, J=6 Hz, 2H), 6.67-6.72 (m, 2H), 7.70 (d, J=10 Hz, 1H).

### Preparation 9

### 2-[4-(2-Methoxyethoxy)-2-methylphenyl]-4,4,5,5-tetramethyl-[1,3,2]dioxaborolane

### a) 1-Bromo-4-(2-methoxyethoxy)-2-methylbenzene

To a solution of 4-bromo-3-methylphenol (502 mg, 2.68 mmol) in 3 mL of methanol were added 1-chloro-2-methoxyethane (0.74 mL, 7.9 mmol) and potassium carbonate (364 mg, 2.64 mmol) and the mixture was heated in a microwave system (“Initiator sixty” from Biotage®) at 140° C. for 30 minutes. The reaction was filtered through a pad of Celite® washing with ethyl acetate. The solvent was removed under reduced pressure to give the title compound (663 mg, 99%) as an oil.

### b) 2-[4-(2-Methoxyethoxy)-2-methylphenyl]-4,4,5,5-tetramethyl-[1,3,2]dioxaborolane

In a Schlenk tube a mixture of the title compound from Preparation 9a (770 mg, 3.14 mmol), 4,4,5,5,4′,4′,5′,5′-Octamethyl-[2,2′]bi[[1,3,2]dioxaborolanyl] (1.22 g, 4.73 mmol), [1,1′-bis(diphenyl-phosphino)ferrocene]dichloropalladium(II) complex with dichloromethane (1:1) (255 mg, 0.31 mmol) and potassium acetate (1.56 g, 15.9 mmol) in N,N-dimethyl formamide (10 mL) was heated at 80° C. for 18 hours. The cooled mixture was diluted with ethyl acetate, washed with water, brine and dried over sodium sulphate. The solvent was removed under reduced pressure and the residue was purified by column chromatography on silica flash, using hexane/ethyl acetate (7:1) as eluents, to yield the title compound (589 mg, 64%).

H¹-NMR δ (CDCl₃): 1.30 (s, 12H), 2.49 (s, 3H), 3.43 (s, 3H), 3.70-3.75 (m, 2H), 4.08-4.13 (m, 2H), 6.67-6.72 (m, 2H), 7.69 (d, J=10 Hz, 1H).

### Preparation 10

### 3-Methyl-4-(4,4,5,5-tetramethyl-[1,3,2]dioxaborolan-2-yl)-benzoic acid

A mixture of 4-bromo-3-methylbenzoic acid (1 g, 4.65 mmol), 4,4,5,5,4′,4′,5′,5′-Octamethyl-[2,2′]bi[[1,3,2]dioxaborolanyl] (1.77 g, 6.97 mmol), [1,1′-bis(diphenylphosphino)ferrocene]dichloropalladium(II) complex with dichloromethane (1:1) (379 mg, 0.46 mmol) and potassium acetate (2.3 g, 23.2 mmol) in N,N-dimethyl formamide (23 mL) was heated in a microwave system (“Initiator sixty” from Biotage®) at 120° C. for 15 minutes. The solvent was evaporated under reduced pressure and the residue suspended in a HCl 2N/ethyl acetate mixture (40 mL, 1:1 v/v). It was filtered through sintered glass and the aqueous phase extracted with ethyl acetate. The combined organic layers were washed with brine, dried over sodium sulphate and the solvent removed under reduced pressure. The residue was purified by column chromatography on silica flash, using hexane/ethyl acetate (7/3) as eluents, to yield the title compound 1.1 g, 90%) as a white solid.

H¹-NMR δ (CDCl₃): 1.36 (s, 12H), 2.59 (s, 3H), 7.85-7.90 (m, 3H).

### Preparation 11

### (3-Amino-2-bromopyridin-4-yl)(2-methoxyphenyl)methanone

### a) N-{4-[Hydroxy(2-methoxyphenyl)methyl]pyridin-3-yl}-2,2-dimethylpropanamide

nBuLi (2.5M in hexanes, 56.2 mL, 140.5 mmol) was dropwise added to a solution of the title compound of Preparation 1a (10 g, 56.2 mmol) and N,N,N′,N′-tetramethylethylenediamine (TMEDA) (20.9 mL, 140.5 mmol) in diethyl ether (338 mL) at −78° C. under argon and the resulting mixture was stirred at that temperature for 15 minutes and at −10° C. for 2 hours. Then, the reaction mixture was cooled down to −78° C. and 2-methoxybenzaldehyde (19.52 g, 140.5 mmol) in 34 mL of dry tetrahydrofuran was carefully added. After 15 minutes, the cooling bath was removed and the mixture stirred overnight at room temperature. Subsequently, water (100 mL) was added to the flask and it was extracted with ethyl acetate (3×200 mL), the organic solution was washed with brine, dried over sodium sulphate and the solvent removed under reduced pressure. The residue was purified by column chromatography on silica flash, using hexane/ethyl acetate (4:1) as eluents, to yield the title compound (11.1 g, 63%) as a solid.

### b) N-[4-(2-Methoxybenzoyl)pyridin-3-yl]-2,2-dimethylpropanamide

Obtained as a yellow solid (99%) from the title compound of Preparation 11a following the experimental procedure described in Preparation 1c.

### c) (3-Aminopyridin-4-yl)(2-methoxyphenyl)methanone

Obtained as a bright yellow solid (85%) from the title compound of Preparation 11b following the experimental procedure described in Preparation 1d.

### d) (3-Amino-1-oxidopyridin-4-yl)(2-methoxyphenyl)methanone

Obtained as a bright yellow solid (80%) from the title compound of Preparation 11c following the experimental procedure described in Preparation 1e.

### e) (3-Amino-2-bromopyridin-4-yl)(2-methoxyphenyl)methanone

Obtained as a bright yellow solid (61%) from the title compound of Preparation 11d following the experimental procedure described in Preparation 1f.

¹H-NMR δ (CDCl₃): 3.75 (s, 3H), 6.79 (brs, 2H), 6.98-7.10 (m, 2H), 7.04 (d, J=6 Hz, 1H), 7.27-7.31 (m, 1H), 7.45-7.52 (m, 1H), 7.62 (d, J=6 Hz, 1H).

### Preparation 12

### (3-Amino-2-bromopyridin-4-yl)(2-chloro-4-fluorophenyl)methanone

### a) N-{4-[(2-Chloro-4-fluorophenyl)(hydroxy)methyl]pyridin-3-yl}-2,2-dimethylpropanamide

nBuLi (2.5M in hexanes, 56.2 mL, 140.5 mmol) was dropwise added to a solution of the title compound of Preparation 1a (10 g, 56.2 mmol) and N,N,N′,N′-tetramethylethylenediamine (TMEDA) (20.9 mL, 140.5 mmol) in diethyl ether (338 mL) at −78° C. under argon and the resulting mixture was stirred at that temperature for 15 minutes and at −10° C. for 2 hours. Then, the reaction mixture was cooled down to −78° C. and 2-chloro-4-fluorobenzaldehyde (20 g, 140.5 mmol) in 34 mL of dry tetrahydrofuran was carefully added. After 15 minutes, the cooling bath was removed and the mixture stirred overnight at room temperature. Subsequently, water (100 mL) was added to the flask and it was extracted with ethyl acetate (3×200 mL), the organic solution was washed with brine, dried over sodium sulphate and the solvent removed under reduced pressure. The residue was purified by column chromatography on silica flash, using dichloromethane/ethyl acetate (7:3) as eluents, to yield the title compound (6.15 g, 33%) as a solid.

### b) N-[4-(2-Chloro-4-fluorobenzoyl)pyridin-3-yl]-2,2-dimethylpropanamide

Obtained as a yellow solid (99%) from the title compound of Preparation 22a following the experimental procedure described in Preparation 1c.

### c) (3-Aminopyridin-4-yl)(2-chloro-4-fluorophenyl)methanone

Obtained as a bright yellow solid (92%) from the title compound of Preparation 22b following the experimental procedure described in Preparation 1d.

### d) (3-Amino-1-oxidopyridin-4-yl)(2-chloro-4-fluorophenyl)methanone

Obtained as a bright yellow solid (83%) from the title compound of Preparation 22c following the experimental procedure described in Preparation 1e.

### e) (3-Amino-2-bromopyridin-4-yl)(2-chloro-4-fluorophenyl)methanone

Obtained as a bright yellow solid (46%) from the title compound of Preparation 22d following the experimental procedure described in Preparation 1f.

¹H-NMR δ (CDCl₃): 6.88 (brs, 2H), 6.96 (d, J=6 Hz, 1H), 7.08-7.17 (m, 1H), 7.23 (dd, J=2 and 8 Hz, 1H), 7.34 (dd, J=6 and 10 Hz, 1H), 7.65 (d, J=6 Hz, 1H).

### EXAMPLES

TABLE 2

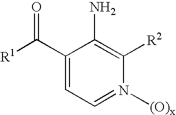

| Example | R¹ | R² | x |
|---|---|---|---|
| 1 | 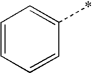 | 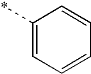 | 0 |
| 2 | 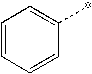 | 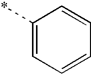 | 1 |
| 3 | 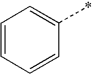 | 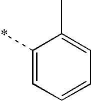 | 0 |
| 4 | 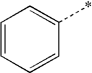 | 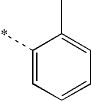 | 1 |
| 5 | 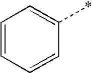 | 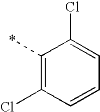 | 0 |
| 6 | 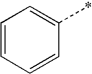 | 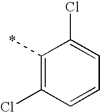 | 1 |
| 7 | 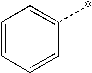 | 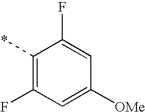 | 0 |
| 8 | 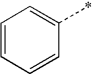 | 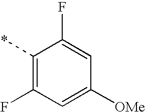 | 1 |
| 9 | 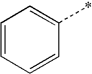 | 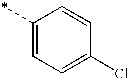 | 0 |
| 10 | 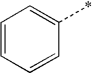 | 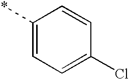 | 1 |
| 11 | 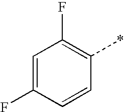 | 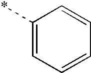 | 0 |
| 12 | 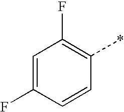 | 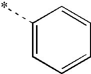 | 1 |
| 13 | 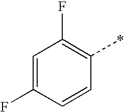 | 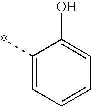 | 0 |
| 14 | 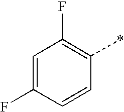 | 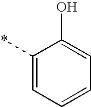 | 1 |
| 15 | 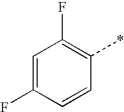 | 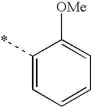 | 0 |
| 16 | 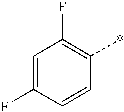 | 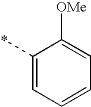 | 1 |
| 17 | 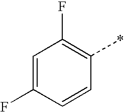 | 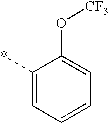 | 0 |
| 18 | 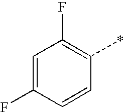 | 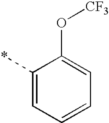 | 1 |
| 19 | 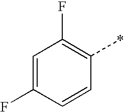 | 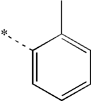 | 0 |
| 20 | 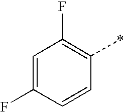 | 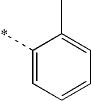 | 1 |
| 21 | 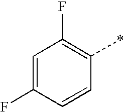 | 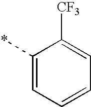 | 0 |
| 22 | 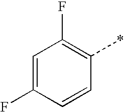 | 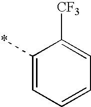 | 1 |
| 23 | 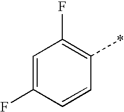 | 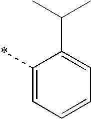 | 0 |
| 24 | 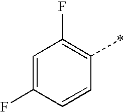 | 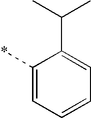 | 1 |
| 25 | 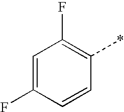 | 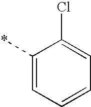 | 0 |
| 26 | 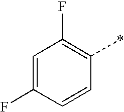 | 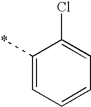 | 1 |
| 27 | 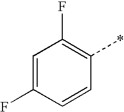 | 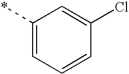 | 0 |
| 28 | 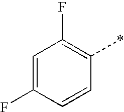 | 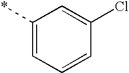 | 1 |
| 29 | 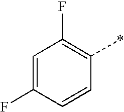 | 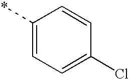 | 0 |
| 30 | 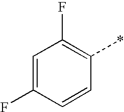 | 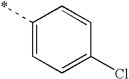 | 1 |
| 31 | 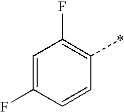 | 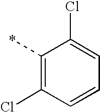 | 0 |
| 32 | 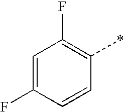 | 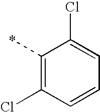 | 1 |
| 33 | 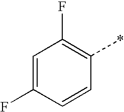 | 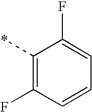 | 0 |
| 34 | 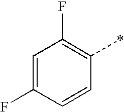 | 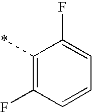 | 1 |
| 35 | 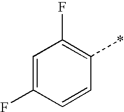 | 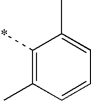 | 0 |
| 36 | 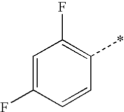 | 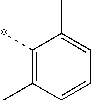 | 1 |
| 37 | 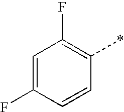 | 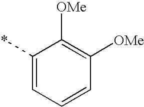 | 0 |
| 38 | 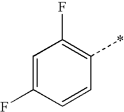 | 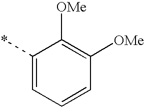 | 1 |
| 39 | 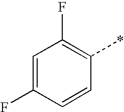 | 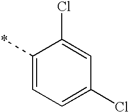 | 0 |
| 40 | 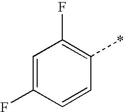 | 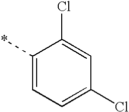 | 1 |
| 41 | 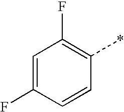 | 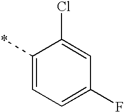 | 0 |
| 42 | 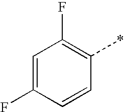 | 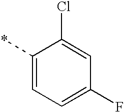 | 1 |
| 43 | 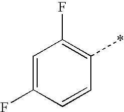 | 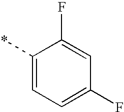 | 0 |
| 44 | 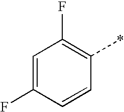 | 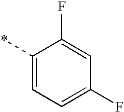 | 1 |
| 45 | 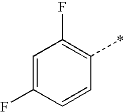 | 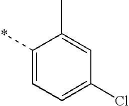 | 0 |
| 46 | 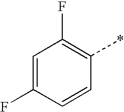 | 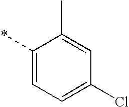 | 1 |
| 47 | 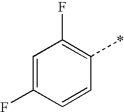 | 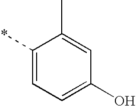 | 0 |
| 48 | 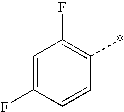 | 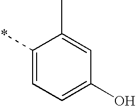 | 1 |
| 49 | 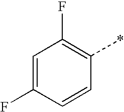 | 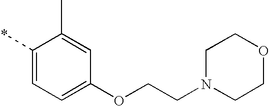 | 0 |
| 50 | 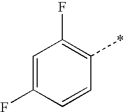 | 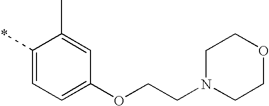 | 1 |
| 51 | 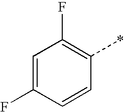 | 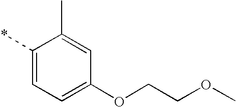 | 0 |
| 52 | 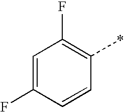 | 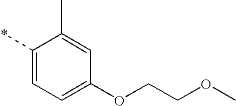 | 1 |
| 53 | 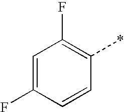 | 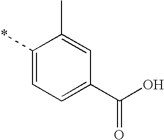 | 0 |
| 54 | 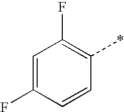 | 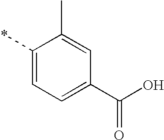 | 1 |
| 55 | 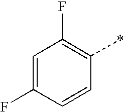 | 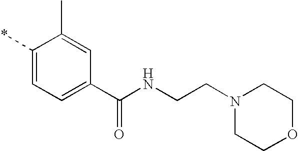 | 0 |
| 56 | 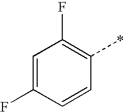 | 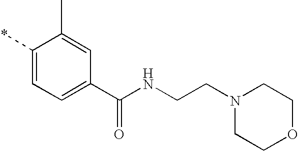 | 1 |
| 57 | 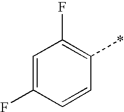 | 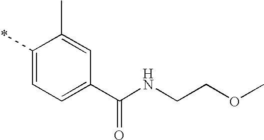 | 0 |
| 58 | 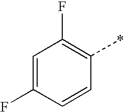 | 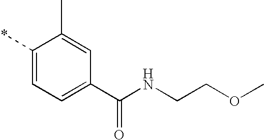 | 1 |
| 59 | 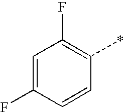 | 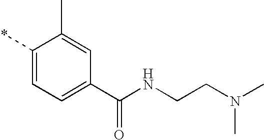 | 0 |
| 60 | 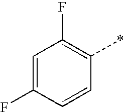 | 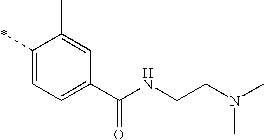 | 1 |
| 61 | 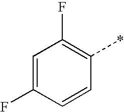 | 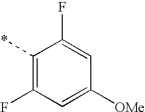 | 0 |
| 62 | 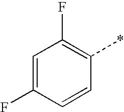 | 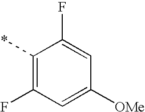 | 1 |
| 63 | 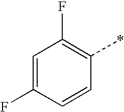 | 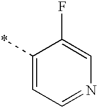 | 0 |
| 64 | 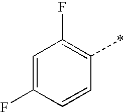 | 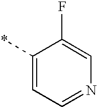 | 1 |
| 65 | 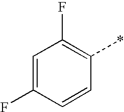 | 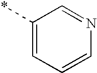 | 0 |
| 66 | 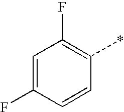 | 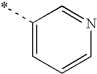 | 1 |
| 67 | 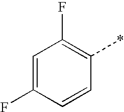 | 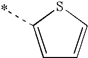 | 0 |
| 68 | 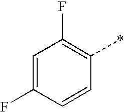 | 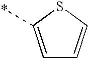 | 1 |
| 69 | 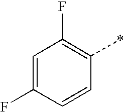 | 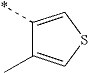 | 0 |
| 70 | 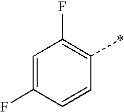 | 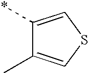 | 1 |
| 71 | 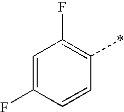 | 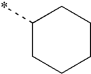 | 0 |
| 72 | 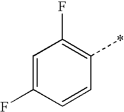 | 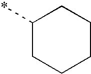 | 1 |
| 73 | 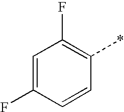 | 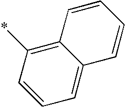 | 0 |
| 74 | 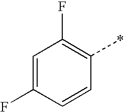 | 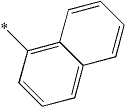 | 1 |
| 75 | 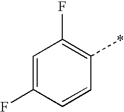 | 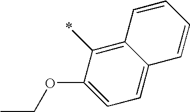 | 0 |
| 76 | 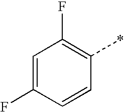 | 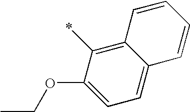 | 1 |
| 77 | 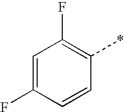 | 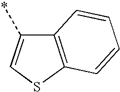 | 0 |
| 78 | 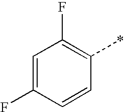 | 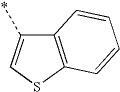 | 1 |
| 79 | 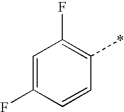 | 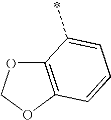 | 0 |
| 80 | 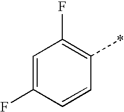 | 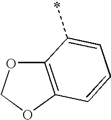 | 1 |
| 81 | 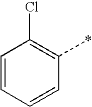 | 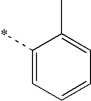 | 0 |
| 82 | 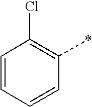 | 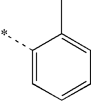 | 1 |
| 83 | 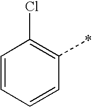 | 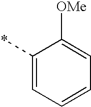 | 0 |
| 84 | 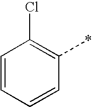 | 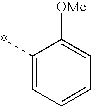 | 1 |
| 85 | 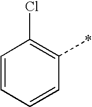 | 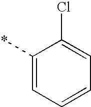 | 0 |
| 86 | 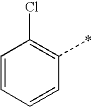 | 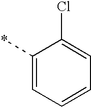 | 1 |
| 87 | 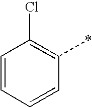 | 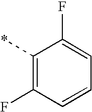 | 0 |
| 88 | 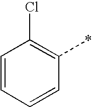 | 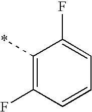 | 1 |
| 89 | 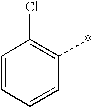 | 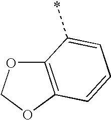 | 0 |
| 90 | 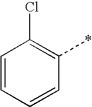 | 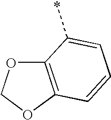 | 1 |
| 91 | 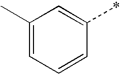 | 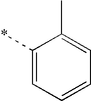 | 0 |
| 92 | 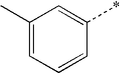 | 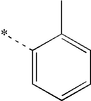 | 1 |
| 93 | 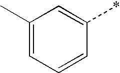 | 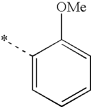 | 0 |
| 94 | 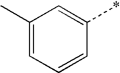 | 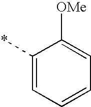 | 1 |
| 95 | 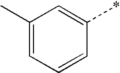 | 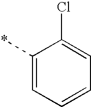 | 0 |
| 96 | 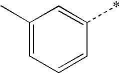 | 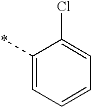 | 1 |
| 97 | 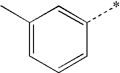 | 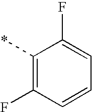 | 0 |
| 98 | 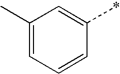 | 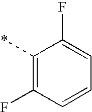 | 1 |
| 99 | 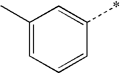 | 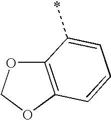 | 0 |
| 100 | 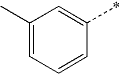 | 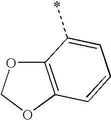 | 1 |
| 101 | 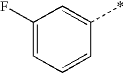 | 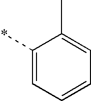 | 0 |
| 102 | 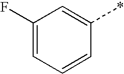 | 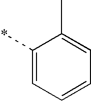 | 1 |
| 103 | 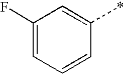 | 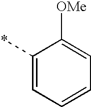 | 0 |
| 104 | 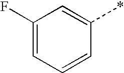 | 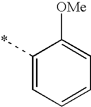 | 1 |
| 105 | 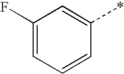 | 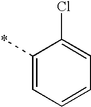 | 0 |
| 106 | 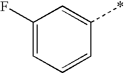 | 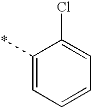 | 1 |
| 107 | 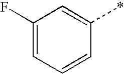 | 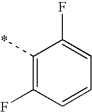 | 0 |
| 108 | 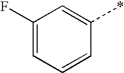 | 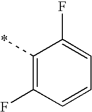 | 1 |
| 109 | 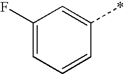 | 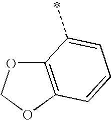 | 0 |
| 110 | 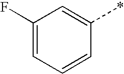 | 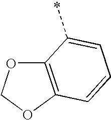 | 1 |
| 111 | 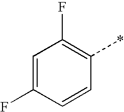 | 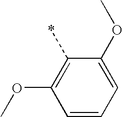 | 0 |
| 112 | 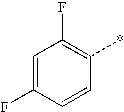 | 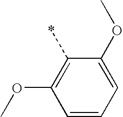 | 1 |
| 113 | 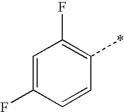 | 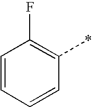 | 0 |
| 114 | 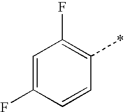 | 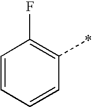 | 1 |
| 115 | 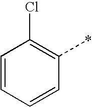 | 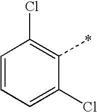 | 0 |
| 116 | 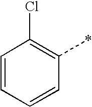 | 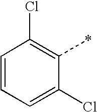 | 1 |
| 117 | 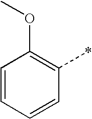 | 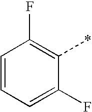 | 0 |
| 118 | 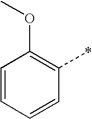 | 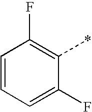 | 1 |
| 119 | 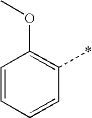 | 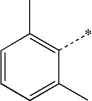 | 0 |
| 120 | 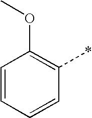 | 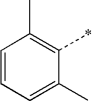 | 1 |
| 121 | 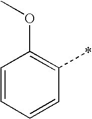 | 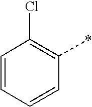 | 0 |
| 122 | 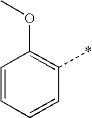 | 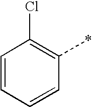 | 1 |
| 123 | 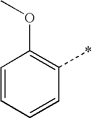 | 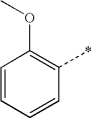 | 0 |
| 124 | 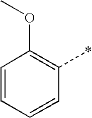 | 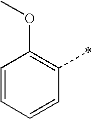 | 1 |
| 125 | 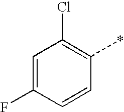 | 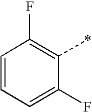 | 0 |
| 126 | 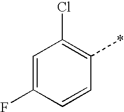 | 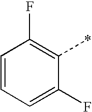 | 1 |
| 127 | 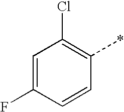 | 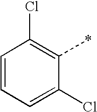 | 0 |
| 128 | 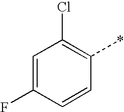 | 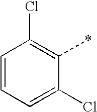 | 1 |
| 129 | 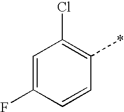 | 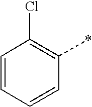 | 0 |
| 130 | 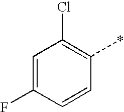 | 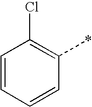 | 1 |

### Example 1

### (3-Amino-2-phenylpyridin-4-yl)(phenyl)methanone

In a Schlenk tube were charged the compound of Preparation 1 (50 mg, 0.18 mmol), phenylboronic acid (29 mg, 0.23 mmol), potassium carbonate (50 mg, 0.36 mmol) and the solvents dimethoxyethane (1 ml) and water (0.07 ml), and a nitrogen atmosphere was established. Then tetrakis(triphenylphosphine)palladium(0) (10 mg, 0.009 mmol) was added and this mixture was stirred under nitrogen for 24 h at 80° C. Subsequently, water was added to the cold reaction mixture and it was extracted with ethyl acetate (3×50 ml), the organic solution washed with brine, dried (Na₂SO₄), and the solvent removed under reduced pressure. The residue was purified by column chromatography on silica flash and hexane/ethyl acetate (3/1) as eluents to yield the title compound (35 mg, 72%) as a yellow solid.

LRMS (m/z): 275 (M+1)⁺.

Retention Time: 14 min.

H¹-NMR δ (CDCl₃): 6.1 (brs, 2H), 7.21 (d, J=6.7 Hz, 2H), 7.4-7.81 (m, 9H), 8.07 (d, J=5.3 Hz, 1H).

### Example 2

### (3-Amino-1-oxido-2-phenylpyridin-4-yl)(phenyl)methanone

To a solution of Example 1 (137 mg, 0.5 mmol) in dichloromethane (3 ml) at 0° C. was portionwise added meta-chloroperbenzoic acid (130 mg, 0.75 mmol) and the reaction mixture was stirred overnight at room temperature. Then, more dichloromethane was added (30 ml) and the solution was washed with aqueous sodium bicarbonate 4% (3×30 ml) and brine. The organic layer was dried over sodium sulphate and concentrated under reduced pressure to give a residue that was purified by crystallization from a mixture of hexane, diethyl ether and ethyl acetate to yield the title compound (113 mg, 75%) as a yellow solid.

LRMS (m/z): 291 (M+1)⁺.

Retention Time: 12 min.

H¹-NMR δ (CDCl₃): 6.32 (brs, 2H), 7.36 (d, J=6.0 Hz, 1H), 7.45-7.68 (m, 12H).

### Example 3

### [3-Amino-2-(2-methylphenyl)pyridin-4-yl](phenyl)methanone

In a Schlenk tube were charged the compound of Preparation 1 (101 mg, 0.37 mmol), 2-methylphenyl boronic acid (75 mg, 0.55 mmol), cesium carbonate (2M aqueous solution, 0.55 mL, 1.098 mmol) and dioxane (1 mL). The mixture was submitted to three vacuum-argon cycles, then [1,1′-bis(diphenylphosphino)ferrocene]dichloropalladium(II) complex with dichloromethane (1:1) (21 mg, 0.026 mmol) was added and the mixture purged in the same way. The reaction was stirred at 80° C. under argon for 17 h. Subsequently, water was added to the cold reaction mixture and it was extracted with ethyl acetate (3×50 ml), the organic solution was washed with brine, dried over sodium sulphate and the solvent removed under reduced pressure. The residue was purified by column chromatography on silica flash, using hexane/ethyl acetate (5/1) as eluents, to yield the title compound (82 mg, 78%) as a yellow solid.

LRMS (m/z): 289 (M+1)⁺.

Retention Time: 15 min.

H¹-NMR δ (CDCl₃): 2.23 (s, 3H), 5.83 (brs, 2H), 7.33 (m, 4H), 7.47-7.8 (m, 5H), 8.03 (d, J=5.3 Hz, 1H).

### Example 4

### [3-Amino-2-(2-methylphenyl)-1-oxidopyridin-4-yl](phenyl)methanone

Obtained as a yellow solid (96%) from the title compound of Example 3 following the procedure of Example 2.

LRMS (m/z): 305 (M+1)⁺.

Retention Time: 13 min.

H¹-NMR δ (CDCl₃): 2.23 (s, 3H), 6.27 (brs, 2H), 7.23-7.73 (m, 11H).

### Example 5

### [3-Amino-2-(2,6-dichlorophenyl)pyridin-4-yl](phenyl)methanone

In a Schlenk tube were charged the compound of Preparation 1 (100 mg, 0.36 mmol), 2,6-dichlorophenyl boronic acid (137 mg, 0.72 mmol), potassium carbonate (229 mg, 1.08 mmol) and toluene (3 mL). The mixture was submitted to three vacuum-argon cycles, then S—PHOS (9 mg, 0.022 mmol) and tris(dibenzylideneacetone)dipalladium(0) (10 mg, 0.011 mmol) was added and the mixture purged in the same way. The reaction was stirred at 100° C. under argon for 2 days. Subsequently, water was added to the cold reaction mixture and it was extracted with ethyl acetate (3×50 ml), the organic solution was washed with brine, dried over sodium sulphate and the solvent removed under reduced pressure. The residue was purified by column chromatography on silica flash, using hexane/ethyl acetate (6/1) as eluents, to yield the title compound (44 mg, 36%) as a yellow solid.

LRMS (m/z): 343, 345, 347 (M+1)⁺.

Retention Time: 15 min.

H¹-NMR δ (CDCl₃): 5.73 (brs, 2H), 7.25-7.8 (m, 9H), 8.13 (d, J=5.3 Hz, 1H).

### Example 6

### [3-Amino-2-(2,6-dichlorophenyl)-1-oxidopyridin-4-yl](phenyl)methanone

Obtained as a yellow solid (90%) from the title compound of Example 5 following the procedure of Example 2.

LRMS (m/z): 359, 361, 363 (M+1)⁺.

Retention Time: 13 min.

H¹-NMR δ (CDCl₃): 6.14 (brs, 2H), 7.36-7.65 (m, 10H).

### Example 7

### [3-Amino-2-(2,6-difluoro-4-methoxyphenyl)pyridin-4-yl](phenyl)methanone

Obtained as a yellow solid (10%) from the title compound of Preparation 1 and 2,6-difluoro-4-methoxyphenylboronic acid following the experimental procedure described in Example 3 (24 h at 80° C.).

LRMS (m/z): 341 (M+1)⁺.

Retention Time: 15 min.

H¹-NMR δ (CDCl₃): 3.90 (s, 3H), 5.90 (brs, 2H), 6.67 (m, 2H), 7.33 (d, J=8.0 Hz, 1H), 7.50-7.80 (m, 5H), 8.10 (d, J=6.7 Hz, 1H).

### Example 8

### [3-Amino-2-(2,6-difluoro-4-methoxyphenyl)-1-oxidopyridin-4-yl](phenyl)methanone

Obtained as a yellow solid (52%) from the title compound of Example 7 following the procedure of Example 2.

LRMS (m/z): 357 (M+1)⁺.

Retention Time: 13 min.

H¹-NMR δ (CDCl₃): 3.90 (s, 3H), 6.33 (brs, 2H), 6.70 (m, 2H), 7.40-7.73 (m, 7H).

### Example 9

### [3-Amino-2-(4-chlorophenyl)pyridin-4-yl](phenyl)methanone

Obtained as a yellow solid (71%) from the title compound of Preparation 1 and 4-chlorophenylboronic acid following the experimental procedure described in Example 3 (17 h at 80° C.).

LRMS (m/z): 309, 311 (M+1)⁺.

Retention Time: 16 min.

H¹-NMR δ (CDCl₃): 6.07 (brs, 2H), 7.26 (m, 1H), 7.46-7.8 (m, 9H), 8.07 (d, J=5.0 Hz, 1H).

### Example 10

### [3-Amino-2-(4-chlorophenyl)-1-oxidopyridin-4-yl](phenyl)methanone

Obtained as a yellow solid (100%) from the title compound of Example 9 following the procedure of Example 2.

LRMS (m/z): 325, 327 (M+1)⁺.

Retention Time: 13 min.

H¹-NMR δ (CDCl₃): 6.3 (brs, 2H), 7.33-7.73 (m, 11H).

### Example 11

### (3-Amino-2-phenylpyridin-4-yl)(2,4-difluorophenyl)methanone

Obtained as a yellow solid (83%) from the title compound of Preparation 2 and phenylboronic acid following the experimental procedure described in Example 3 (17 h at 80° C.).

LRMS (m/z): 311 (M+1)⁺.

Retention Time: 15 min.

H¹-NMR δ (CDCl₃): 6.49 (brs, 2H), 6.90-7.13 (m, 3H), 7.46-7.58 (m, 4H), 7.64-7.69 (m, 2H), 8.01 (d, J=5.0 Hz, 1H).

### Example 12

### (3-Amino-1-oxido-2-phenylpyridin-4-yl)(2,4-difluorophenyl)methanone

Obtained as a yellow solid (53%) from the title compound of Example 11 following the procedure of Example 2.

LRMS (m/z): 327 (M+1)⁺.

Retention Time: 13 min.

H¹-NMR δ (CDCl₃): 6.49 (brs, 2H), 6.91-7.08 (m, 2H), 7.17 (dd, J=7.0 and 3.1 Hz, 1H), 7.45-7.65 (m, 7H).

### Example 13

### [3-Amino-2-(2-hydroxyphenyl)pyridin-4-yl](2,4-difluorophenyl)methanone

Obtained as a yellow solid (78%) from the title compound of Preparation 2 and 2-hydroxyphenylboronic acid following the experimental procedure described in Example 3 (17 h at 80° C.).

LRMS (m/z): 327 (M+1)⁺.

Retention Time: 14 min.

H¹-NMR δ (CDCl₃): 6.81 (brs, 2H), 6.90-7.16 (m, 5H), 7.34 (m, 1H), 7.55 (m, 1H), 7.87 (dd, J=7.8 and 1.5 Hz, 1H), 7.93 (d, J=5.1 Hz, 1H).

### Example 14

### [3-Amino-2-(2-hydroxyphenyl)-1-oxidopyridin-4-yl](2,4-difluorophenyl)methanone

Obtained as a yellow solid (13%) from the title compound of Example 13 following the procedure of Example 2.

LRMS (m/z): 343 (M+1)⁺.

Retention Time: 12 min.

H¹-NMR δ (CDCl₃): 6.49 (brs, 2H), 6.92-7.13 (m, 4H), 7.23-7.29 (m, 2H), 7.46-7.57 (m, 3H), 7.72 (d, J=7.0 Hz, 1H).

### Example 15

### [3-Amino-2-(2-methoxyphenyl)pyridin-4-yl](2,4-difluorophenyl)methanone

Obtained as a yellow solid (81%) from the title compound of Preparation 2 and 2-methoxyphenylboronic acid following the experimental procedure described in Example 3 (17 h at 80° C.).

LRMS (m/z): 341 (M+1)⁺.

Retention Time: 14 min.

H¹-NMR δ (CDCl₃): 3.85 (s, 3H), 6.30 (brs, 2H), 6.91-7.14 (m, 5H), 7.37-7.57 (m, 3H), 8.00 (d, J=5.5 Hz, 1H).

### Example 16

### [3-Amino-2-(2-methoxyphenyl)-1-oxidopyridin-4-yl](2,4-difluorophenyl)methanone

Obtained as a yellow solid (79%) from the title compound of Example 15 following the procedure of Example 2.

LRMS (m/z): 357 (M+1)⁺.

Retention Time: 13 min.

H¹-NMR δ (CDCl₃): 3.85 (s, 3H), 6.45 (brs, 2H), 6.91-7.19 (m, 5H), 7.31 (m, 1H), 7.44-7.57 (m, 2H), 7.62 (d, J=7 Hz, 1H).

### Example 17

### {3-Amino-2-[2-(trifluoromethoxy)phenyl]pyridin-4-yl}(2,4-difluorophenyl)methanone

Obtained as a yellow solid (64%) from the title compound of Preparation 2 and 2-(trifluoromethoxy)phenylboronic acid following the experimental procedure described in Example 3 (24 h at 80° C.).

LRMS (m/z): 395 (M+1)⁺.

Retention Time: 17 min.

H¹-NMR δ (CDCl₃): 6.16 (brs, 2H), 6.91-7.09 (m, 2H), 7.17 (dd, J=5.4, and 2.9 Hz, 1H), 7.42-7.60 (m, 5H), 8.04 (d, J=5.5 Hz, 1H).

### Example 18

### 3-Amino-1-oxido-2-[2-(trifluoromethoxy)phenyl]pyridin-4-yl}(2,4-difluorophenyl)methanone

Obtained as a yellow solid (63%) from the title compound of Example 17 following the procedure of Example 2.

LRMS (m/z): 411 (M+1)⁺.

Retention Time: 14 min.

H¹-NMR δ (CDCl₃): 6.41 (brs, 2H), 6.92-7.11 (m, 2H), 7.20-7.24 (m, 1H), 7.44-7.61 (m, 5H), 7.65 (d, J=7.0 Hz, 1H).

### Example 19

### [3-Amino-2-(2-methylphenyl)pyridin-4-yl](2,4-difluorophenyl)methanone

Obtained as a yellow solid (66%) from the title compound of Preparation 2 and 2-methylphenylboronic acid following the experimental procedure described in Example 3 (48 h at 100° C.).

LRMS (m/z): 325 (M+1)⁺.

Retention Time: 16 min.

H¹-NMR δ (CDCl₃): 2.21 (s, 3H), 6.17 (brs, 2H), 6.91-7.09 (m, 2H), 7.13 (dd, J=5.1 and 2.7 Hz, 1H), 7.31-7.37 (m, 4H), 7.48-7.59 (m, 1H), 8.00 (d, J=5.1 Hz).

### Example 20

### [3-Amino-2-(2-methylphenyl)-1-oxidopyridin-4-yl](2,4-difluorophenyl)methanone

Obtained as a yellow solid (95%) from the title compound of Example 19 following the procedure of Example 2.

LRMS (m/z): 341 (M+1)⁺.

Retention Time: 20 min.

H¹-NMR δ (CDCl₃): 2.21 (s, 3H), 6.40 (brs, 2H), 6.91-7.10 (m, 2H), 7.20 (dd, J=7.0 and 3.1 Hz, 1H), 7.29 (m, 1H), 7.35-7.57 (m, 4H), 7.64 (d, J=7.0 Hz, 1H).

### Example 21

### {3-Amino-2-[2-(trifluoromethyl)phenyl]pyridin-4-yl}(2,4-difluorophenyl)methanone

Obtained as a yellow solid (64%) from the title compound of Preparation 2 and 2-(trifluoromethyl)phenylboronic acid following the experimental procedure described in Example 3 (3 days at 100° C.).

LRMS (m/z): 379 (M+1)⁺.

Retention Time: 16 min.

H¹-NMR δ (CDCl₃): 6.02 (brs, 2H), 6.91-7.10 (m, 2H), 7.18 (dd, J=5.5 and 2.8 Hz, 1H), 7.47-7.75 (m, 4H), 7.88 (m, 1H), 8.00 (d, J=5.4 Hz, 1H).

### Example 22

### {3-Amino-1-oxido-2-[2-(trifluoromethyl)phenyl]pyridin-4-yl}(2,4-difluorophenyl)methanone

Obtained as a yellow solid (54%) from the title compound of Example 21 following the procedure of Example 2.

LRMS (m/z): 395 (M+1)⁺.

Retention Time: 14 min.

H¹-NMR δ (CDCl₃): 6.25 (brs, 2H), 6.92-7.11 (m, 2H), 7.22-7.25 (m, 1H), 7.42-7.85 (m, 5H), 7.94 (d, J=7.4 Hz, 1H).

### Example 23

### [3-Amino-2-(2-isopropylphenyl)pyridin-4-yl](2,4-difluorophenyl)methanone

Obtained as a yellow solid (80%) from the title compound of Preparation 2 and 2-isopropylphenylboronic acid following the experimental procedure described in Example 3 (3 days at 100° C.).

LRMS (m/z): 353 (M+1)⁺.

Retention Time: 18 min.

H¹-NMR δ (CDCl₃): 1.16 (d, J=7.1 Hz, 3H), 1.21 (d, J=7.0 Hz, 3H), 2.77 (hept, J=7.1 Hz, 1H), 6.16 (brs, 2H), 6.91-7.09 (m, 2H), 7.13 (dd, J=5.4 and 2.7 Hz, 1H), 7.24-7.59 (m, 5H), 8.00 (d, J=5.5 Hz, 1H).

### Example 24

### [3-Amino-2-(2-isopropylphenyl)-1-oxidopyridin-4-yl](2,4-difluorophenyl)methanone

Obtained as a yellow solid (93%) from the title compound of Example 23 following the procedure of Example 2.

LRMS (m/z): 369 (M+1)⁺.

Retention Time: 16 min.

H¹-NMR δ (CDCl₃): 1.19 (d, J=6.6 Hz, 3H), 1.27 (d, J=7.0 Hz, 3H), 2.59 (hept, J=6.7 Hz, 1H), 6.38 (brs, 2H), 6.92-7.10 (m, 2H), 7.18-7.23 (m, 2H), 7.36-7.58 (m, 4H), 7.66 (d, J=7.4 Hz, 1H).

### Example 25

### [3-Amino-2-(2-chlorophenyl)pyridin-4-yl](2,4-difluorophenyl)methanone

Obtained as a yellow solid (78%) from the title compound of Preparation 2 and 2-chlorophenylboronic acid following the experimental procedure described in Example 3 (24 h at 100° C.).

LRMS (m/z): 345, 347 (M+1)⁺.

Retention Time: 16 min.

H¹—NMR δ (CDCl₃): 6.02 (brs, 2H), 6.92-7.10 (m, 2H), 7.18 (dd, J=5.5 and 2.8 Hz, 1H), 7.47-7.75 (m, 4H), 7.88 (m, 1H), 8.00 (d, J=5.4 Hz, 1H).

### Example 26

### [3-Amino-2-(2-chlorophenyl)-1-oxidopyridin-4-yl](2,4-difluorophenyl)methanone

Obtained as a yellow solid (72%) from the title compound of Example 25 following the procedure of Example 2.

LRMS (m/z): 361, 363 (M+1)⁺.

Retention Time: 13 min.

H¹-NMR δ (CDCl₃): 6.38 (brs, 2H), 6.92-7.10 (m, 2H), 7.24 (dd, J=7.0 and 2.8 Hz, 1H), 7.38-7.58 (m, 4H), 7.62-7.67 (m, 2H).

### Example 27

### [3-Amino-2-(3-chlorophenyl)pyridin-4-yl](2,4-difluorophenyl)methanone

Obtained as a yellow solid (71%) from the title compound of Preparation 2 and 3-chlorophenylboronic acid following the experimental procedure described in Example 3 (17 h at 80° C.).

LRMS (m/z): 345, 347 (M+1)⁺.

Retention Time: 17 min.

H¹-NMR δ (CDCl₃): 6.44 (brs, 2H), 6.90-7.08 (m, 2H), 7.13 (dd, J=5.1 and 2.8 Hz, 1H), 7.43-7.60 (m, 4H), 7.67 (m, 1H), 8.01 (d, J=5.5 Hz, 1H).

### Example 28

### [3-Amino-2-(3-chlorophenyl)-1-oxidopyridin-4-yl](2,4-difluorophenyl)methanone

Obtained as a yellow solid (75%) from the title compound of Example 27 following the procedure of Example 2.

LRMS (m/z): 361, 363 (M+1)⁺.

Retention Time: 14 min.

H¹-NMR δ (CDCl₃): 6.47 (brs, 2H), 6.91-7.10 (m, 2H), 7.20 (dd, J=7.0 and 3.1 Hz, 1H), 7.37 (m, 1H), 7.45-7.56 (m, 4H), 7.63 (d, J=7.0 Hz, 1H).

### Example 29

### [3-Amino-2-(4-chlorophenyl)pyridin-4-yl](2,4-difluorophenyl)methanone

Obtained as a yellow solid (84%) from the title compound of Preparation 2 and 4-chlorophenylboronic acid following the experimental procedure described in Example 3 (17 h at 80° C.).

H¹-NMR δ (CDCl₃): 6.40 (brs, 2H), 6.93-7.13 (m, 3H), 7.50-7.70 (m, 5H), 8.0 (d, J=6.7 Hz, 1H).

### Example 30

### [3-Amino-2-(4-chlorophenyl)-1-oxidopyridin-4-yl](2,4-difluorophenyl)methanone

Obtained as a yellow solid (79%) from the title compound of Example 29 following the procedure of Example 2.

LRMS (m/z): 361, 363 (M+1)⁺.

Retention Time: 14 min.

H¹-NMR δ (CDCl₃): 6.5 (brs, 2H), 6.90-7.10 (m, 2H), 7.20 (dd, J=7.0 and 3.1 Hz, 1H), 7.43-7.70 (m, 6H).

### Example 31

### [3-Amino-2-(2,6-dichlorophenyl)pyridin-4-yl](2,4-difluorophenyl)methanone

Obtained as a yellow solid (28%) from the title compound of Preparation 2 and 2,6-dichlorophenylboronic acid following the experimental procedure described in Example 5 (3 days at 100° C.).

LRMS (m/z): 379, 381, 383 (M+1)⁺.

Retention Time: 16 min.

H¹-NMR δ (CDCl₃): 6.06 (brs, 2H), 6.91-7.10 (m, 2H), 7.23 (dd, J=5.5 and 3.1 Hz, 1H), 7.33-7.61 (m, 4H), 8.09 (d, J=5.4 Hz, 1H).

### Example 32

### [3-Amino-2-(2,6-dichlorophenyl)-1-oxidopyridin-4-yl](2,4-difluorophenyl)methanone

Obtained as a yellow solid (87%) from the title compound of Example 31 following the procedure of Example 2.

LRMS (m/z): 395, 397, 399 (M+1)⁺.

Retention Time: 14 min.

H¹-NMR δ (CDCl₃): 6.35 (brs, 2H), 6.92-7.11 (m, 2H), 7.28 (dd, J=7.4 and 2.7 Hz, 1H), 7.40-7.59 (m, 4H), 7.67 (d, J=7.0 Hz, 1H).

### Example 33

### [3-Amino-2-(2,6-difluorophenyl)pyridin-4-yl](2,4-difluorophenyl)methanone

nBuLi (2.5M in hexanes, 0.56 mL) was dropwise added to a solution of 1,3-difluorobenzene (146 mg, 1.28 mmol) in dry tetrahydrofuran (2 mL) at −78° C. under argon and the resulting mixture was stirred at that temperature for 30 minutes. Then, the reaction mixture was warmed up to −50° C. and ZnCl₂ (0.5M in THF, 2.8 mL) carefully added. After 20 minutes, the title compound from Preparation 2 (200 mg, 0.64 mmol, in 1.5 mL of THF) and tetrakis(triphenylphosphine)palladium(0) (66 mg, 0.06 mmol) were sequentially added. The mixture was then submitted to three vacuum-argon cycles and warmed, first to room temperature for 15 minutes and then to 40° C. for 48 hours. After this time the reaction was cooled down and the solvent evaporated under reduced pressure. The resulting crude was purified by column chromatography on silica flash using hexane/ethyl acetate (8/2 to 7/3) as eluents to yield the title compound (150 mg, 68%) as a yellow solid.

LRMS (m/z): 347 (M+1)⁺.

Retention Time: 15 min.

H¹-NMR δ (CDCl₃): 6.20 (brs, 2H), 6.93-7.14 (m, 4H), 7.22 (dd, J=5.4 and 3.1 Hz, 1H), 7.39-7.59 (m, 2H), 8.08 (d, J=5.5 Hz, 1H).

### Example 34

### [3-Amino-2-(2,6-difluorophenyl)-1-oxidopyridin-4-yl](2,4-difluorophenyl)methanone

Obtained as a yellow solid (73%) from the title compound of Example 33 following the procedure of Example 2.

LRMS (m/z): 363 (M+1)⁺.

Retention Time: 13 min.

H¹-NMR δ (CDCl₃): 6.49 (brs, 2H), 6.92-7.17 (m, 4H), 7.27 (m, 1H), 7.46-7.60 (m, 2H), 7.67 (d, J=7.1 Hz, 1H).

### Example 35

### [3-Amino-2-(2,6-dimethylphenyl)pyridin-4-yl](2,4-difluorophenyl)methanone

Obtained as a yellow solid (44%) from the title compound of Preparation 2 and 2,6-dimethylphenylboronic acid following the experimental procedure described in Example 5 (2 days at 100° C.).

LRMS (m/z): 339 (M+1)⁺.

Retention Time: 17 min.

H¹-NMR δ (CDCl₃): 2.08 (s, 6H), 6.08 (brs, 2H), 6.91-7.30 (m, 6H), 7.55 (m, 1H), 8.04 (d, J=5.5 Hz, 1H).

### Example 36

### [3-Amino-2-(2,6-dimethylphenyl)-1-oxidopyridin-4-yl](2,4-difluorophenyl)methanone

Obtained as a yellow solid (73%) from the title compound of Example 35 following the procedure of Example 2.

LRMS (m/z): 355 (M+1)⁺.

Retention Time: 14 min.

H¹-NMR δ (CDCl₃): 2.14 (s, 6H), 6.35 (brs, 2H), 6.92-7.10 (m, 2H), 7.19-7.38 (m, 4H), 7.53 (m, 1H), 7.67 (d, J=7.1 Hz, 1H).

### Example 37

### [3-Amino-2-(2,3-dimethoxyphenyl)pyridin-4-yl](2,4-difluorophenyl)methanone

Obtained as a yellow solid (84%) from the title compound of Preparation 2 and 2,3-dimethoxyphenylboronic acid following the experimental procedure described in Example 3 (17 h at 100° C.).

LRMS (m/z): 371 (M+1)⁺.

Retention Time: 15 min.

H¹-NMR δ (CDCl₃): 3.71 (s, 3H), 3.94 (s, 3H), 6.36 (brs, 2H), 6.90-7.09 (m, 4H), 7.14 (dd, J=5.5 and 3.2 Hz, 1H), 7.20 (d, J=8.2 Hz, 1H), 7.53 (m, 1H), 8.00 (d, J=5.1 Hz, 1H).

### Example 38

### [3-Amino-2-(2,3-dimethoxyphenyl)-1-oxidopyridin-4-yl](2,4-difluorophenyl)methanone

Obtained as a yellow solid (67%) from the title compound of Example 37 following the procedure of Example 2.

LRMS (m/z): 387 (M+1)⁺.

Retention Time: 13 min.

H¹-NMR δ (CDCl₃): 3.84 (s, 3H), 3.94 (s, 3H), 6.44 (brs, 2H), 6.85-7.13 (m, 4H), 7.19 (dd, J=7.0 and 2.7 Hz, 1H), 7.24-7.32 (m, 1H), 7.51 (m, 1H), 7.64 (d, J=7.0 Hz, 1H).

### Example 39

### [3-Amino-2-(2,4-dichlorophenyl)pyridin-4-yl](2,4-difluorophenyl)methanone

Obtained as a yellow solid (24%) from the title compound of Preparation 2 and 2,4-dichlorophenylboronic acid following the experimental procedure described in Example 3 (18 h at 85° C.).

LRMS (m/z): 379-381-383 (M+1)⁺.

Retention Time: 17 min.

H¹-NMR δ (CDCl₃): 6.12 (brs, 2H), 6.91-7.07 (m, 2H), 7.20 (dd, J=2 and 4 Hz, 1H), 7.40-7.60 (m, 4H), 8.03 (d, J=6 Hz, 1H).

### Example 40

### [3-Amino-2-(2,4-dichlorophenyl)-1-oxidopyridin-4-yl](2,4-difluorophenyl)methanone

Obtained as a yellow solid (47%) from the title compound of Example 39 following the procedure of Example 2.

LRMS (m/z): 395-397-399 (M+1)⁺.

Retention Time: 15 min.

H¹-NMR δ (CDCl₃): 6.36 (brs, 2H), 6.90-7.10 (m, 2H), 7.25 (m, 1H), 7.35 (d, J=8 Hz, 1H), 7.45-7.70 (m, 4H).

### Example 41

### [3-Amino-2-(2-chloro-4-fluorophenyl)pyridin-4-yl](2,4-difluorophenyl)methanone

Obtained as a yellow solid (15%) from the title compound of Preparation 2 and 2-chloro-4-fluorophenylboronic acid following the experimental procedure described in Example 3 (18 h at 85° C.).

LRMS (m/z): 363-365 (M+1)⁺.

Retention Time: 16 min.

H¹-NMR δ (CDCl₃): 6.13 (brs, 2H), 6.90-7.10 (m, 2H), 7.15-7.22 (m, 2H), 7.32 (dd, J=4 and 8 Hz, 1H), 7.40-7.60 (m, 2H), 8.03 (d, J=4 Hz, 1H).

### Example 42

### [3-Amino-2-(2-chloro-4-fluorophenyl)-1-oxidopyridin-4-yl](2,4-difluorophenyl)methanone

Obtained as a yellow solid (17%) from the title compound of Example 41 following the procedure of Example 2.

LRMS (m/z): 379-381 (M+1)⁺.

Retention Time: 14 min.

H¹-NMR δ (CDCl₃): 6.37 (brs, 2H), 6.90-7.10 (m, 2H), 7.18-7.28 (m, 2H), 7.36-7.58 (m, 3H), 7.65 (d, J=8 Hz, 1H)

### Example 43

### [3-Amino-2-(2,4-difluorophenyl)pyridin-4-yl](2,4-difluorophenyl)methanone

Obtained as a yellow solid (32%) from the title compound of Preparation 2 and 2,4-difluorophenylboronic acid following the experimental procedure described in Example 3 (18 h at 85° C.).

LRMS (m/z): 347 (M+1)⁺.

Retention Time: 16 min.

H¹-NMR δ (CDCl₃): 6.25 (brs, 2H), 6.90-7.11 (m, 4H), 7.18 (dd, J=2 and 4 Hz, 1H), 7.46-7.59 (m, 2H), 8.03 (d, J=6 Hz, 1H).

### Example 44

### [3-Amino-2-(2,4-difluorophenyl)-1-oxidopyridin-4-yl](2,4-difluorophenyl)methanone

Obtained as a yellow solid (90%) from the title compound of Example 43 following the procedure of Example 2.

LRMS (m/z): 363 (M+1)⁺.

Retention Time: 13 min.

H¹-NMR δ (CDCl₃): 6.47 (brs, 2H), 6.91-7.15 (m, 4H), 7.24 (dd, J=2 and 4 Hz, 1H), 7.38-7.57 (m, 2H), 7.65 (d, J=8 Hz, 1H).

### Example 45

### [3-Amino-2-(4-chloro-2-methylphenyl)pyridin-4-yl](2,4-difluorophenyl)methanone

Obtained as a yellow solid (83%) from the title compound of Preparation 2 and 4-chloro-2-methylphenylboronic acid following the experimental procedure described in Example 3 (18 h at 85° C.).

LRMS (m/z): 359 (M+1)⁺.

Retention Time: 17 min.

H¹-NMR δ (CDCl₃): 2.19 (s, 3H), 6.12 (brs, 2H), 6.90-7.25 (m, 2H), 7.15 (dd, J=2 and 4 Hz, 1H), 7.25-7.36 (m, 3H), 7.49-7.60 (m, 1H), 8.00 (d, J=6 Hz, 1H).

### Example 46

### [3-Amino-2-(4-chloro-2-methylphenyl)-1-oxidopyridin-4-yl](2,4-difluorophenyl)methanone

Obtained as a yellow solid (84%) from the title compound of Example 45 following the procedure of Example 2.

LRMS (m/z): 375 (M+1)⁺.

Retention Time: 15 min.

H¹-NMR δ (CDCl₃): 2.20 (s, 3H), 6.37 (brs, 2H), 6.90-7.10 (m, 2H), 7.19-7.25 (m, 2H), 7.36-7.57 (m, 3H), 7.64 (d, J=8 Hz, 1H).

### Example 47

### [3-Amino-2-(4-hydroxy-2-methylphenyl)pyridin-4-yl](2,4-difluorophenyl)methanone

Obtained as a yellow solid (58%) from the title compound of Preparation 2 and the title compound of Preparation 6 following the experimental procedure described in Example 3 (18 h at 85° C.).

LRMS (m/z): 341 (M+1)⁺.

Retention Time: 14 min.

H¹-NMR δ (CDCl₃): 2.08 (s, 3H), 6.25 (brs, 2H), 6.58-6.62 (m, 2H), 6.90-7.10 (m, 3H), 7.18 (dd, J=2 and 4 Hz, 1H), 7.48-7.59 (m, 1H), 7.97 (d, J=6 Hz, 1H).

### Example 48

### [3-Amino-2-(4-hydroxy-2-methylphenyl)-1-oxidopyridin-4-yl](2,4-difluorophenyl)methanone

Obtained as a yellow solid (99%) from the title compound of Example 47 following the procedure of Example 2.

LRMS (m/z): 357 (M+1)⁺.

Retention Time: 13 min.

H¹-NMR δ (DMSO-d₆): 1.94 (s, 3H), 6.70-6.80 (m, 2H), 6.88 (brs, 2H), 6.97 (d, J=8 Hz, 1H), 7.14 (dd, J=2 and 8 Hz, 1H), 7.20-7.30 (m, 1H), 7.40-7.50 (m, 1H), 7.51 (d, J=8 Hz, 1H), 7.60-7.70 (m, 1H).

### Example 49

### [3-Amino-2-(4-[2-(3-methylphenoxy)ethyl]morpholino)pyridin-4-yl](2,4-difluorophenyl)methanone

Obtained as a yellow solid (61%) from the title compound of Preparation 2 the title compound of Preparation 8 following the experimental procedure described in Example 3 (18 h at 100° C.).

LRMS (m/z): 454 (M+1)⁺.

Retention Time: 10 min.

H¹-NMR δ (CDCl₃): 2.18 (s, 3H), 2.62 (t, J=6 Hz, 4H), 2.85 (t, J=6 Hz, 2H), 3.77 (t, J=6 Hz, 4H), 4.18 (t, J=6 Hz, 2H), 6.19 (brs, 2H), 6.84-7.04 (m, 4H), 7.10 (dd, J=2 and 4 Hz, 1H), 7.24 (d, J=4 Hz, 1H), 7.47-7.58 (m, 1H), 7.98 (d, J=4 Hz, 1H).

### Example 50

### [3-Amino-2-(4-[2-(3-methylphenoxy)ethyl]morpholino)-1-oxidopyridin-4-yl](2,4-difluoro-phenyl)methanone

To a solution of the title compound from Example 48 (200 mg, 0.56 mmol) in 6 mL of acetonitrile were added 4-(2-chloroethyl)morpholine hydrochloride (156 mg, 0.84 mmol) and potassium carbonate (301 mg, 2.18 mmol) and the mixture was heated to 80° C. for 18 h. The reaction was cooled down and filtered through a pad of Celite® washing with acetonitrile (10 mL). The solvent was removed under reduced pressure to give a crude oil which was purified by column chromatography on silica flash using dichloromethane/methanol (95/5) as eluents. The resulting solid was further purified by crystallization from a mixture of diisopropylether and ethyl acetate (2/1) to yield the title compound (142 mg, 54%) as a bright yellow solid.

LRMS (m/z): 470 (M+1)⁺.

Retention Time: 8.9 min.

H¹-NMR δ (CDCl₃): 2.17 (s, 3H), 2.61 (t, J=6 Hz, 4H), 2.84 (t, J=6 Hz, 2H), 3.76 (t, J=6 Hz, 4H), 4.17 (t, J=6 Hz, 2H), 6.43 (brs, 2H), 6.90-7.10 (m, 4H), 7.14-7.21 (m, 2H), 7.45-7.56 (m, 1H), 7.62 (d, J=6 Hz, 1H).

### Example 51

### {3-Amino-2-[4-(2-methoxyethoxy)-2-methylphenyl]pyridin-4-yl}(2,4-difluorophenyl)methanone

Obtained as a yellow solid (42%) from the title compound of Preparation 2 and the title compound of Preparation 9 following the experimental procedure described in Example 3 (18 h at 100° C.).

LRMS (m/z): 399 (M+1)⁺.

Retention Time: 16 min.

H¹—NMR δ (CDCl₃): 2.18 (s, 3H), 3.48 (s, 3H), 3.79 (t, J=6 Hz, 2H), 4.18 (t, J=6 Hz, 2H), 6.20 (brs, 2H), 6.87-7.04 (m, 4H), 7.10 (dd, J=2 and 4 Hz, 1H), 7.25 (d, J=6 Hz, 1H), 7.47-7.58 (m, 1H), 7.98 (d, J=6 Hz, 1H).

### Example 52

### {3-Amino-2-[4-(2-methoxyethoxy)-2-methylphenyl]-1-oxidopyridin-4-yl}(2,4-difluorophenyl)methanone

Obtained as a yellow solid (72%) from the title compound of Example 51 following the procedure of Example 2.

LRMS (m/z): 415 (M+1)⁺.

Retention Time: 14 min.

H¹-NMR δ (CDCl₃): 2.17 (s, 3H), 3.47 (s, 3H), 3.78 (t, J=4 Hz, 2H), 4.18 (t, J=4 Hz, 2H), 6.43 (brs, 2H), 6.91-7.01 (m, 4H), 7.14-7.21 (m, 2H), 7.45-7.56 (m, 1H), 7.63 (d, J=8 Hz, 1H).

### Example 53

### 4-[3-Amino-4-(2,4-difluorobenzoyl)pyridin-2-yl]-3-methylbenzoic acid

Obtained as a solid (60%) from the title compound of Preparation 2 and the title compound of Preparation 10 following the experimental procedure described in Example 3 (18 h at 100° C.).

LRMS (m/z): 369 (M+1)⁺.

Retention Time: 15 min.

H¹-NMR δ (CD₃OD): 2.23 (s, 3H), 7.10-7.20 (m, 2H), 7.26 (dd, J=2 and 4 Hz, 1H), 7.42 (d, J=8 Hz, 1H), 7.58-7.70 (m, 1H), 7.88 (d, J=6 Hz, 1H), 8.00-8.09 (m, 2H).

### Example 54

### 4-[3-Amino-4-(2,4-difluorobenzoyl)-1-oxidopyridin-2-yl]-3-methylbenzoic acid

Obtained as a yellow solid (57%) from the title compound of Example 53 following the procedure of Example 2.

LRMS (m/z): 385 (M+1)⁺.

Retention Time: 13 min.

H¹-NMR δ (DMSO-d₆): 7.01 (brs, 2H), 7.24-7.70 (m, 6H), 7.89-7.98 (m, 2H).

### Example 55

### 4-[3-Amino-4-(2,4-difluorobenzoyl)pyridin-2-yl]-3-methyl-N-(2-morpholin-4-ylethyl)benzamide

To a solution of the title compound from Example 53 (50 mg, 0.14 mmol) in 2 mL of N,N-dimethylformamide were added (2-morpholin-4-ylethyl)amine (25 mg, 0.19 mmol), O-benzotriazol-1-yl-N,N,N′,N′-tetramethyluronium hexafluorophosphate (HBTU) (65 mg, 0.17 mmol) and diisopropyl ethyl amine (301 mg, 2.18 mmol) and the mixture was stirred overnight under argon. The reaction was diluted with ethyl acetate, washed with 5% citric acid, water, brine and dried over sodium sulphate. Removal of the solvent under reduced pressure afforded a residue which was purified by column chromatography on silica flash, using dichloromethane/ethanol (95/5) as eluents, to yield the title compound (30 mg, 45%) as a bright yellow solid.

LRMS (m/z): 481 (M+1)⁺.

Retention Time: 9 min.

H¹-NMR δ (CDCl₃): 2.27 (s, 3H), 2.56 (t, J=4 Hz, 4H), 2.66 (t, J=6 Hz, 2H), 3.60 (dd, J=6 and 12 Hz, 2H), 3.76 (t, J=4 Hz, 4H), 6.10 (bs, 2H), 6.80-7.10 (m, 3H), 7.16 (dd, J=2 and 4 Hz, 1H), 7.43 (d, J=6 Hz, 1H), 7.49-7.60 (m, 1H), 7.70 (d, J=8 Hz, 1H), 7.81 (brs, 1H), 8.03 (d, J=6 Hz, 1H).

### Example 56

### 4-[3-Amino-4-(2,4-difluorobenzoyl)-1-oxidopyridin-2-yl]-3-methyl-N-(2-morpholin-4-ylethyl)benzamide

Obtained as a yellow solid (1%) from the title compound of Example 54 and (2-morpholin-4-ylethyl)amine following the procedure of Example 55.

LRMS (m/z): 497 (M+1)⁺.

Retention Time: 8.5 min.

H¹-NMR δ (CD₃OD): 2.23 (s, 3H), 2.64-2.75 (m, 6H), 3.61 (t, J=8 Hz, 2H), 3.72-3.77 (m, 4H), 7.12-7.21 (m, 2H), 7.36-7.43 (m, 2H), 7.56-7.68 (m, 2H), 7.83-7.90 (m, 2H).

### Example 57

### 4-[3-Amino-4-(2,4-difluorobenzoyl)pyridin-2-yl]-3-methyl-N-(2-methoxyethyl)-3-methylbenzamide

Obtained as a yellow solid (73%) from the title compound of Example 53 and (2-methoxyethyl)amine following the procedure of Example 55.

LRMS (m/z): 426 (M+1)⁺.

Retention Time: 14 min.

H¹-NMR δ (CDCl₃): 2.26 (s, 3H), 3.42 (s, 3H), 3.62 (dd, J=4 and 8 Hz, 2H), 3.68 (m, 2H), 6.10 (brs, 2H), 6.58 (brt, J=6 Hz, 1H), 6.90-7.00 (m, 2H), 7.16 (dd, J=4 and 6 Hz, 1H), 7.42 (d, J=6 Hz, 1H), 7.50-7.60 (m, 1H), 7.70-7.80 (m, 2H), 8.02 (d, J=6 Hz, 1H).

### Example 58

### 4-[3-Amino-4-(2,4-difluorobenzoyl)-1-oxidopyridin-2-yl]-3-methyl-N-(2-methoxyethyl)-3-methylbenzamide

Obtained as a yellow solid (60%) from the title compound of Example 57 following the procedure of Example 2.

LRMS (m/z): 442 (M+1)⁺.

Retention Time: 12 min.

H¹-NMR δ (CDCl₃): 2.26 (s, 3H), 3.41 (s, 3H), 3.60 (dd, J=4 and 8 Hz, 2H), 3.67 (m, 2H), 6.34 (brs, 2H), 6.57 (brt, J=6 Hz, 1H), 6.92-7.10 (m, 2H), 7.23 (dd, J=4 and 8 Hz, 1H), 7.37 (d, J=8 Hz, 1H), 7.49-7.60 (m, 1H), 7.65 (d, J=8 Hz, 1H), 7.75-7.84 (m, 2H).

### Example 59

### 4-[3-Amino-4-(2,4-difluorobenzoyl)pyridin-2-yl]-3-methyl-N-[2-(dimethylamino)ethyl]-3-methylbenzamide

Obtained as a yellow solid (58%) from the title compound of Example 53 and N,N-dimethylethane-1,2-diamine following the procedure of Example 55.

LRMS (m/z): 439 (M+1)⁺.

Retention Time: 9 min.

H¹-NMR δ (CDCl₃): 2.26 (s, 3H), 2.30 (s, 6H), 2.56 (t, J=6 Hz, 2H), 3.52-3.60 (m, 2H), 6.11 (brs, 2H), 6.90-7.10 (m, 3H), 7.15 (dd, J=4 and 6 Hz, 1H), 7.42 (d, J=8 Hz, 1H), 7.50-7.60 (m, 1H), 7.70-7.82 (m, 2H), 8.02 (d, J=6 Hz, 1H).

### Example 60

### 4-[3-Amino-4-(2,4-difluorobenzoyl)-1-oxidopyridin-2-yl]-3-methyl-N-[2-(dimethylamino)ethyl]-3-methylbenzamide

Obtained as a yellow solid (3%) from the title compound of Example 54 and N,N-dimethylethane-1,2-diamine following the procedure of Example 55.

LRMS (m/z): 455 (M+1)⁺.

Retention Time: 8 min.

H¹-NMR δ (CD₃OD): 2.23 (s, 3H), 2.99 (s, 6H), 3.39 (m, 2H), 3.79 (m, 2H), 7.13-7.22 (m, 2H), 7.39-7.44 (m, 2H), 7.57-7.69 (m, 2H), 7.86-7.94 (m, 2H).

### Example 61

### [3-Amino-2-(2,6-difluoro-4-methoxyphenyl)pyridin-4-yl](2,4-difluorophenyl)methanone

Obtained as a yellow solid (21%) from the title compound of Preparation 2 and 2-bromo-1,3-difluoro-5-methoxybenzene following the experimental procedure described in Example 33.

LRMS (m/z): 377 (M+1)⁺.

Retention Time: 16 min.

H¹-NMR δ (CDCl₃): 3.86 (s, 3H), 6.23 (brs, 2H), 6.62 (m, 2H), 6.90-7.00 (m, 2H), 7.19 (dd, J=2 and 4 Hz, 1H), 7.47-7.58 (m, 1H), 8.05 (d, J=6 Hz, 1H).

### Example 62

### [3-Amino-2-(2,6-difluoro-4-methoxyphenyl)-1-oxidopyridin-4-yl](2,4-difluorophenyl)methanone

Obtained as a yellow solid (72%) from the title compound of Example 61 following the procedure of Example 2.

LRMS (m/z): 393 (M+1)⁺.

Retention Time: 14 min.

H¹-NMR δ (CDCl₃): ): 3.88 (s, 3H), 6.54 (brs, 2H), 6.63-6.72 (m, 2H), 6.92-7.10 (m, 2H), 7.23 (dd, J=4 and 8 Hz, 1H), 7.45-7.56 (m, 1H), 7.65 (d, J=8 Hz, 1H).

### Example 63

### (3-Amino-3′-fluoro-2,4′-bipyridin-4-yl)(2,4-difluorophenyl)methanone

Obtained as a solid (22%) from the title compound of Preparation 2 and 3-fluoro-4-pyridyl boronic acid following the experimental procedure described in Example 3 (18 h at 100° C.).

LRMS (m/z): 330 (M+1)⁺.

Retention Time: 14 min.

H¹-NMR δ (CDCl₃): ): 6.24 (brs, 2H), 6.90-7.12 (m, 2H), 7.24 (dd, J=4 and 6 Hz, 1H), 7.49-7.60 (m, 2H), 8.08 (d, J=6 Hz, 1H), 8.61 (dd, J=2 and 4 Hz, 1H), 8.66 (d, J=2 Hz, 1H).

### Example 64

### [3-Amino-2-(3-fluoropyridin-4-yl)-1-oxidopyridin-4-yl](2,4-difluorophenyl)methanone

Obtained as a solid (26%) from the title compound of Example 63 following the procedure of Example 2.

LRMS (m/z): 346 (M+1)⁺.

Retention Time: 12 min.

H¹-NMR δ (CDCl₃): 6.45 (brs, 2H), 6.92-7.12 (m, 2H), 7.30 (dd, J=4 and 6 Hz, 1H), 7.42-7.58 (m, 2H), 7.67 (d, J=6 Hz, 1H), 8.67 (d, J=4 Hz, 1H), 8.74 (bs, 1H).

### Example 65

### (3-Amino-2,3′-bipyridin-4-yl)(2,4-difluorophenyl)methanone

Obtained as a solid (76%) from the title compound of Preparation 2 and 3-pyridyl boronic acid following the experimental procedure described in Example 3 (18 h at 100° C.).

LRMS (m/z): 312 (M+1)⁺.

Retention Time: 12 min.

H¹-NMR δ (CDCl₃): 6.39 (brs, 2H), 6.90-7.10 (m, 2H), 7.18 (dd, J=4 and 6 Hz, 1H), 7.46-7.60 (m, 2H), 8.00-8.05 (m, 1H), 8.06 (d, J=6 Hz, 1H), 8.72 (bs, 1H), 8.97 (bs, 1H).

### Example 66

### (3-Amino-1-oxido-2-pyridin-3-ylpyridin-4-yl)(2,4-difluorophenyl)methanone

Obtained as a solid (7%) from the title compound of Example 65 following the procedure of Example 2.

LRMS (m/z): 328 (M+1)⁺.

Retention Time: 10 min.

H¹-NMR δ (CDCl₃): 6.49 (brs, 2H), 6.90-7.10 (m, 2H), 7.24 (dd, J=4 and 8 Hz, 1H), 7.45-7.60 (m, 2H), 7.66 (d, J=8 Hz, 1H), 7.85-7.91 (m, 1H), 8.74 (d, J=2 Hz, 1H), 8.78 (dd, J=2 and 6 Hz, 1H).

### Example 67

### [3-Amino-2-(2-thienyl)pyridin-4-yl](2,4-difluorophenyl)methanone

Obtained as a solid (61%) from the title compound of Preparation 2 and thiophene-2-boronic acid following the experimental procedure described in Example 3 (18 h at 80° C.).

LRMS (m/z): 317 (M+1)⁺.

Retention Time: 16 min.

H¹-NMR δ (CDCl₃): 6.74 (brs, 2H), 6.88-7.10 (m, 3H), 7.17-7.21 (m, 1H), 7.47-7.60 (m, 3H), 7.99 (d, J=6 Hz, 1H).

### Example 68

### [3-Amino-1-oxido-2-(2-thienyl)pyridin-4-yl](2,4-difluorophenyl)methanone

Obtained as a solid (28%) from the title compound of Example 67 following the procedure of Example 2.

LRMS (m/z): 333 (M+1)⁺.

Retention Time: 13 min.

H¹-NMR δ (CDCl₃): 6.90-7.10 (m, 2H), 7.15 (dd, J=4 and 8 Hz, 1H), 7.25 (dd, J=4 and 6 Hz, 1H), 7.44-7.56 (m, 2H), 7.63 (d, J=8 Hz, 1H), 7.69 (dd, J=2 and 4 Hz, 1H).

### Example 69

### [3-Amino-2-(4-methyl-3-thienyl)pyridin-4-yl](2,4-difluorophenyl)methanone

Obtained as a solid (79%) from the title compound of Preparation 2 and 4-methyl-3-thiophene boronic acid following the experimental procedure described in Example 3 (18 h at 100° C.).

LRMS (m/z): 331 (M+1)⁺.

Retention Time: 16 min.

H¹-NMR δ (CDCl₃): 2.20 (s, 3H), 6.37 (brs, 2H), 6.90-7.08 (m, 2H), 7.09-7.15 (m, 2H), 7.47-7.58 (m, 2H), 8.99 (d, J=6 Hz, 1H).

### Example 70

### [3-Amino-2-(4-methyl-3-thienyl)-1-oxidopyridin-4-yl](2,4-difluorophenyl)methanone

Obtained as a solid (99%) from the title compound of Example 69 following the procedure of Example 2.

LRMS (m/z): 347 (M+1)⁺.

Retention Time: 13 min.

H¹-NMR δ (CDCl₃): 2.15 (s, 3H), 6.58 (brs, 2H), 6.90-7.10 (m, 2H), 7.20 (dd, J=6 and 8 Hz, 1H), 7.23 (m, 1H), 7.45-7.55 (m, 2H), 7.61 (d, J=8 Hz, 1H).

### Example 71

### (3-Amino-2-cyclohexylpyridin-4-yl)(2,4-difluorophenyl)methanone

In a 5 mL Biotage® vial were placed the title compound from Preparation 2 (313 mg, 1 mmol), bis-(triphenylphosphin)-palladium (II) chloride (7 mg, 0.01 mmol) and copper (I) iodide (6 mg, 0.033 mmol). The vial was encapsulated and cyclohexylzinc bromide (0.5M in THF, 3 mL) was carefully added. The reaction mixture was heated under microwave conditions (using the system “Initiator sixty” from Biotage) at 120° C. for 20 min. Then, the solvent was evaporated and the residue purified by column chromatography (C-18 reverse phase Biotage® cartridge (water (0.1% acetic acid)/acetonitrile 95:5 to 5:95)) to give the title compound as a solid (11 mg, 3% yield).

LRMS (m/z): 317 (M+1)⁺.

Retention Time: 17 min.

H¹-NMR δ (CDCl₃): 1.25-1.96 (m, 10H), 2.65-2.80 (m, 1H), 6.40 (brs, 2H), 6.85-7.05 (m, 3H), 7.41-7.52 (m, 1H), 7.90 (d, J=4H, 1H).

### Example 72

### (3-Amino-2-cyclohexyl-1-oxidopyridin-4-yl)(2,4-difluorophenyl)methanone

Obtained as a solid (36%) from the title compound of Example 71 following the procedure of Example 2.

LRMS (m/z): 333 (M+1)⁺.

Retention Time: 15 min.

### Example 73

### [3-Amino-2-(1-naphthyl)pyridin-4-yl](2,4-difluorophenyl)methanone

Obtained as a solid (87%) from the title compound of Preparation 2 and 1-naphthalene boronic acid following the experimental procedure described in Example 3 (18 h at 85° C.).

LRMS (m/z): 361 (M+1)⁺.

Retention Time: 17 min.

H¹-NMR δ (CDCl₃): 6.17 (brs, 2H), 6.93-7.11 (m, 2H), 7.23 (dd, J=2 and 4 Hz, 1H), 7.43-7.67 (m, 6H), 7.90-8.03 (m, 2H), 8.10 (d, J=6 Hz, 1H).

### Example 74

### [3-Amino-2-(1-naphthyl)-1-oxidopyridin-4-yl](2,4-difluorophenyl)methanone

Obtained as a solid (90%) from the title compound of Example 73 following the procedure of Example 2.

LRMS (m/z): 377 (M+1)⁺.

Retention Time: 15 min.

H¹-NMR δ (CDCl₃): 6.39 (brs, 2H), 6.94-7.12 (m, 2H), 7.28 (dd, J=2 and 6 Hz, 1H), 7.45-7.71 (m, 6H), 7.72 (d, J=6 Hz, 1H), 7.95-8.07 (m, 2H).

### Example 75

### [3-Amino-2-(2-ethoxy-1-naphthyl)pyridin-4-yl](2,4-difluorophenyl)methanone

Obtained as a solid (21%) from the title compound of Preparation 2 and 2-ethoxy-1-naphthalene boronic acid following the experimental procedure described in Example 3 (18 h at 85° C.).

LRMS (m/z): 405 (M+1)⁺.

Retention Time: 17 min.

H¹—NMR δ (CDCl₃): 1.27 (t, J=6 Hz, 3H), 4.18 (q, J=6 Hz, 2H), 6.14 (brs, 2H), 6.94-7.10 (m, 2H), 7.18-7.42 (m, 5H), 7.52-7.63 (m, 1H), 7.83-7.88 (m, 1H), 7.97 (d, J=8 Hz, 1H), 8.11 (d, J=4 Hz, 1H).

### Example 76

### [3-Amino-2-(2-ethoxy-1-naphthyl)-1-oxidopyridin-4-yl](2,4-difluorophenyl)methanone

Obtained as a solid (53%) from the title compound of Example 75 following the procedure of Example 2.

LRMS (m/z): 421 (M+1)⁺.

Retention Time: 16 min.

H¹-NMR δ (CDCl₃): 1.32 (t, J=8 Hz, 3H), 4.22 (q, J=8 Hz, 2H), 6.38 (brs, 2H), 6.93-7-10 (m, 2H), 7.23-7.46 (m, 5H), 7.48-7.61 (m, 1H), 7.70 (d, J=8 Hz, 1H), 7.85-7.90 (m, 1H), 8.02 (d, J=10 Hz, 1H).

### Example 77

### [3-Amino-2-(1-benzothien-3-yl)pyridin-4-yl](2,4-difluorophenyl)methanone

Obtained as a yellow solid (62%) from the title compound of Preparation 2 and 1-benzothiophen-3-ylboronic acid following the experimental procedure described in Example 3 (18 h at 85° C.).

LRMS (m/z): 367 (M+1)⁺.

Retention Time: 17 min.

H¹-NMR δ (CDCl₃): 6.45 (brs, 2H), 6.92-7.10 (m, 2H), 7.18 (dd, J=2 and 4 Hz, 1H), 7.40-7.44 (m, 2H), 7.50-7.61 (m, 1H), 7.76-7.81 (m, 1H), 7.80 (s, 1H), 7.93-7.98 (m, 1H), 8.08 (d, J=6 Hz, 1H).

### Example 78

### [3-Amino-2-(1-benzothien-3-yl)-1-oxidopyridin-4-yl](2,4-difluorophenyl)methanone

Obtained as a yellow solid (18%) from the title compound of Example 77 following the procedure of Example 2.

LRMS (m/z): 383 (M+1)⁺.

Retention Time: 15 min.

H¹-NMR δ (CDCl₃): 6.62 (brs, 2H), 6.92-7.11 (m, 2H), 7.25 (dd, J=4 and 6 Hz, 1H), 7.36-7.58 (m, 4H), 7.67 (d, J=6 Hz, 1H), 7.78 (s, 1H), 7.92-7.98 (m, 1H).

### Example 79

### [3-Amino-2-(1,3-benzodioxol-4-yl)pyridin-4-yl](2,4-difluorophenyl)methanone

Obtained as a yellow solid (56%) from the title compound of Preparation 2 and the title compound of Preparation 7 following the experimental procedure described in Example 3 (18 h at 100° C.).

LRMS (m/z): 355 (M+1)⁺.

Retention Time: 15 min.

H¹-NMR δ (CDCl₃): 6.06 (s, 2H), 6.8 (brs, 2H), 6.90-7.11 (m, 5H), 7.16 (dd, J=2 and 6 Hz, 1H), 7.46-7.57 (m, 1H), 8.05 (d, J=6 Hz, 1H).

### Example 80

### [3-Amino-2-(1,3-benzodioxol-4-yl)-1-oxidopyridin-4-yl](2,4-difluorophenyl)methanone

Obtained as a yellow solid (43%) from the title compound of Example 79 following the procedure of Example 2.

LRMS (m/z): 371 (M+1)⁺.

Retention Time: 13 min.

H¹-NMR δ (CDCl₃): 6.06 (dd, J=2 and 12 Hz, 2H), 6.62 (brs, 2H), 6.88-7.09 (m, 5H), 7.19 (dd, J=4 and 8 Hz, 1H), 7.43-7.55 (m, 1H), 7.64 (d, J=8 Hz, 1H).

### Example 81

### [3-Amino-2-(2-methylphenyl)pyridin-4-yl](2-chlorophenyl)methanone

Obtained as a yellow solid (43%) from the title compound of Preparation 3 and 2-methylphenyl boronic acid following the experimental procedure described in Example 3 (18 h at 100° C.).

LRMS (m/z): 323-325 (M+1)⁺.

Retention Time: 16 min.

H¹-NMR δ (CDCl₃): 2.21 (s, 3H), 6.30 (brs, 2H), 6.99 (d, J=4 Hz, 1H), 7.33-7.51 (m, 8H), 7.95 (d, J=4 Hz, 1H).

### Example 82

### [3-Amino-2-(2-methylphenyl)-1-oxidopyridin-4-yl](2-chlorophenyl)methanone

Obtained as a yellow solid (51%) from the title compound of Example 81 following the procedure of Example 2.

LRMS (m/z): 339-341 (M+1)⁺.

Retention Time: 14 min.

H¹-NMR δ (CDCl₃): 2.22 (s, 3H), 6.45 (brs, 2H), 7.04 (d, J=6 Hz, 1H), 7.27-7.32 (m, 1H), 7.38-7.50 (m, 7H), 7.59 (d, J=8 Hz, 1H).

### Example 83

### [3-Amino-2-(2-methoxyphenyl)pyridin-4-yl](2-chlorophenyl)methanone

Obtained as a yellow solid (77%) from the title compound of Preparation 3 and 2-methoxyphenyl boronic acid following the experimental procedure described in Example 3 (18 h at 100° C.).

LRMS (m/z): 339-341 (M+1)⁺.

Retention Time: 15 min.

H¹-NMR δ (CDCl₃): 3.84 (s, 3H), 6.44 (brs, 2H), 6.97-7.16 (m, 3H), 7.37-7.51 (m, 6H), 7.95 (d, J=, 4 Hz, 1H).

### Example 84

### [3-Amino-2-(2-methoxyphenyl)-1-oxidopyridin-4-yl](2-chlorophenyl)methanone

Obtained as a yellow solid (46%) from the title compound of Example 83 following the procedure of Example 2.

LRMS (m/z): 355-357 (M+1)⁺.

Retention Time: 13 min.

H¹-NMR δ (CDCl₃): 3.84 (s, 3H), 6.53 (brs, 2H), 7.00-7.19 (m, 3H), 7.30-7.59 (m, 7H).

### Example 85

### [3-Amino-2-(2-chlorophenyl)pyridin-4-yl](2-chlorophenyl)methanone

Obtained as a yellow solid (43%) from the title compound of Preparation 3 and 2-chlorophenyl boronic acid following the experimental procedure described in Example 3 (18 h at 100° C.).

LRMS (m/z): 343-345-347 (M+1)⁺.

Retention Time: 16 min.

H¹-NMR δ (CDCl₃): 6.30 (brs, 2H), 7.05 (d, J=6 Hz, 1H), 7.38-7.49 (m, 7H), 7.53-7.59 (m, 1H), 7.98 (d, J=6 Hz, 1H).

### Example 86

### [3-Amino-2-(2-chlorophenyl)-1-oxidopyridin-4-yl](2-chlorophenyl)methanone

Obtained as a yellow solid (48%) from the title compound of Example 85 following the procedure of Example 2.

LRMS (m/z): 359-361-363 (M+1)⁺.

Retention Time: 13 min.

H¹-NMR δ (CDCl₃): 6.47 (brs, 2H), 7.08 (d, J=6 Hz, 1H), 7.37-7.67 (m, 8H), 7.60 (d, J=8 Hz, 1H).

### Example 87

### [3-Amino-2-(2,6-difluorophenyl)pyridin-4-yl](2-chlorophenyl)methanone

Obtained as a yellow solid (46%) from the title compound of Preparation 3 and 1,3-difluorobenzene following the experimental procedure described in Example 33.

LRMS (m/z): 345-347 (M+1)⁺.

Retention Time: 15 min.

H¹-NMR δ (CDCl₃): 6.34 (brs, 2H), 7.05-7.13 (m, 3H), 7.38-7.51 (m, 5H), 8.03 (d, J=6 Hz, 1H).

### Example 88

### [3-Amino-2-(2,6-difluorophenyl)-1-oxidopyridin-4-yl](2-chlorophenyl)methanone

Obtained as a yellow solid (70%) from the title compound of Example 87 following the procedure of Example 2.

LRMS (m/z): 361-363 (M+1)⁺.

Retention Time: 14 min.

H¹-NMR δ (CDCl₃): 6.57 (brs, 2H), 7.09-7.17 (m, 3H), 7.36-7.56 (m, 5H), 7.62 (d, J=6 Hz, 1H).

### Example 89

### [3-Amino-2-(1,3-benzodioxol-4-yl)pyridin-4-yl](2-chlorophenyl)methanone

Obtained as a yellow solid (88%) from the title compound of Preparation 3 and the title compound of Preparation 7 following the experimental procedure described in Example 3 (18 h at 100° C.).

LRMS (m/z): 353-355 (M+1)⁺.

Retention Time: 16 min.

H¹-NMR δ (CDCl₃): 6.06 (s, 2H), 6.64 (brs, 2H), 6.90-7.12 (m, 4H), 7.33-7.52 (m, 4H), 7.99 (d, J=4 Hz, 1H).

### Example 90

### [3-Amino-2-(1,3-benzodioxol-4-yl)-1-oxidopyridin-4-yl](2-chlorophenyl)methanone

Obtained as a yellow solid (70%) from the title compound of Example 89 following the procedure of Example 2 (4 h, 25° C.).

LRMS (m/z): 369-371 (M+1)⁺.

Retention Time: 13 min.

H¹-NMR δ (CDCl₃): 6.07 (d, J=8 Hz, 2H), 6.71 (brs, 2H), 6.89-7.10 (m, 4H), 7.32-7.48 (m, 4H), 7.58 (d, J=6 Hz, 1H).

### Example 91

### [3-Amino-2-(2-methylphenyl)pyridin-4-yl](3-methylphenyl)methanone

Obtained as a yellow solid (88%) from the title compound of Preparation 4 and 2-methylphenyl boronic acid following the experimental procedure described in Example 3 (4 h at 100° C.).

LRMS (m/z): 303 (M+1)⁺.

Retention Time: 16 min.

H¹-NMR δ (CDCl₃): 2.22 (s, 3H), 2.45 (s, 3H), 5.81 (brs, 2H), 7.27-7.42 (m, 7H), 7.50-7.57 (m, 2H), 8.03 (d, J=6 Hz, 1H).

### Example 92

### [3-Amino-2-(2-methylphenyl)-1-oxidopyridin-4-yl](3-methylphenyl)methanone

Obtained as a yellow solid (93%) from the title compound of Example 91 following the procedure of Example 2.

LRMS (m/z): 319 (M+1)⁺.

Retention Time: 15 min.

H¹-NMR δ (CDCl₃): 2.22 (s, 3H), 2.45 (s, 3H), 6.22 (brs, 2H), 7.27-7.50 (m, 9H), 7.67 (d, J=6 Hz, 1H).

### Example 93

### [3-Amino-2-(2-methoxyphenyl)pyridin-4-yl](3-methylphenyl)methanone

Obtained as a yellow solid (67%) from the title compound of Preparation 4 and 2-methoxyphenyl boronic acid following the experimental procedure described in Example 3 (4 h at 100° C.).

LRMS (m/z): 319 (M+1)⁺.

Retention Time: 15 min.

H¹-NMR δ (CDCl₃): 2.45 (s, 3H), 3.84 (s, 3H), 5.92 (brs, 2H), 7.03-7.15 (m, 2H), 7.29-7.55 (m, 7H), 8.04 (d, J=4 Hz, 1H).

### Example 94

### [3-Amino-2-(2-methoxyphenyl)-1-oxidopyridin-4-yl](3-methylphenyl)methanone

Obtained as a yellow solid (80%) from the title compound of Example 93 following the procedure of Example 2 (4 h, 25° C.).

LRMS (m/z): 335 (M+1)⁺.

Retention Time: 14 min.

H¹-NMR δ (CDCl₃): 2.45 (s, 3H), 3.84 (s, 3H), 6.27 (brs, 2H), 7.09-7.19 (m, 2H), 7.29-7.55 (m, 7H), 7.65 (d, J=8 Hz, 1H).

### Example 95

### [3-Amino-2-(2-chlorophenyl)pyridin-4-yl](3-methylphenyl)methanone

Obtained as a yellow solid (54%) from the title compound of Preparation 4 and 2-chlorophenyl boronic acid following the experimental procedure described in Example 3 (18 h at 100° C.).

LRMS (m/z): 323-325 (M+1)⁺.

Retention Time: 16 min.

H¹—NMR δ (CDCl₃): 2.45 (s, 3H), 5.79 (brs, 2H), 7.33 (d, J=4 Hz, 1H), 7.39-7.46 (m, 5H), 7.50-7.59 (m, 3H), 8.06 (d, J=4 Hz, 1H).

### Example 96

### [3-Amino-2-(2-chlorophenyl)-1-oxidopyridin-4-yl](3-methylphenyl)methanone

Obtained as a yellow solid (49%) from the title compound of Example 95 following the procedure of Example 2.

LRMS (m/z): 339-341 (M+1)⁺.

Retention Time: 14 min.

H¹-NMR δ (CDCl₃): 2.45 (s, 3H), 6.20 (brs, 2H), 7.38-7.52 (m, 9H), 7.68 (d, J=8 Hz, 1H).

### Example 97

### [3-Amino-2-(2,6-difluorophenyl)pyridin-4-yl](3-methylphenyl)methanone

Obtained as a yellow solid (36%) from the title compound of Preparation 4 and 1,3-difluorobenzene following the experimental procedure described in Example 33.

LRMS (m/z): 325 (M+1)⁺.

Retention Time: 16 min.

H¹—NMR δ (CDCl₃): 2.45 (s, 3H), 5.83 (brs, 2H), 7.04-7.12 (m, 2H), 7.36 (d, J=4 Hz, 1H), 7.39-7.56 (m, 5H), 8.10 (d, J=6 Hz, 1H).

### Example 98

### [3-Amino-2-(2,6-difluorophenyl)-1-oxidopyridin-4-yl](3-methylphenyl)methanone

Obtained as a yellow solid (73%) from the title compound of Example 97 following the procedure of Example 2.

LRMS (m/z): 341 (M+1)⁺.

Retention Time: 14 min.

H¹-NMR δ (CDCl₃): 2.45 (s, 3H), 6.31 (brs, 2H), 7.09-7.17 (m, 2H), 7.39-7.60 (m, 6H), 7.70 (d, J=8 Hz, 1H).

### Example 99

### [3-Amino-2-(1,3-benzodioxol-4-yl)pyridin-4-yl](3-methylphenyl)methanone

Obtained as a yellow solid (78%) from the title compound of Preparation 4 and the title compound of Preparation 7 following the experimental procedure described in Example 3 (18 h at 100° C.).

LRMS (m/z): 333 (M+1)⁺.

Retention Time: 16 min.

H¹—NMR δ (CDCl₃): 2.44 (s, 3H), 6.05 (s, 2H), 6.08 (brs, 2H), 6.93 (dd, J=2 and 4 Hz, 1H), 7.00 (t, J=8 Hz, 1H), 7.10 (dd, J=2 and 4 Hz, 1H), 7.29 (d, J=6 Hz, 1H), 7.34-7.55 (m, 4H), 8.07 (d, J=6 Hz, 1H).

### Example 100

### [3-Amino-2-(1,3-benzodioxol-4-yl)-1-oxidopyridin-4-yl](3-methylphenyl)methanone

Obtained as a yellow solid (96%) from the title compound of Example 99 following the procedure of Example 2.

LRMS (m/z): 349 (M+1)⁺.

Retention Time: 14 min.

H¹-NMR δ (CDCl₃): 2.44 (s, 3H), 6.06 (d, J=12 Hz, 1H), 6.07 (d, J=12 Hz, 1H), 6.43 (brs, 2H), 6.89-7.08 (m, 3H), 7.36-7.46 (m, 5H), 7.67 (d, J=8 Hz, 1H).

### Example 101

### [3-Amino-2-(2-methylphenyl)pyridin-4-yl](3-fluorophenyl)methanone

Obtained as a yellow solid (64%) from the title compound of Preparation 5 and 2-methylphenyl boronic acid following the experimental procedure described in Example 3 (18 h at 10° C.).

LRMS (m/z): 307 (M+1)⁺.

Retention Time: 15 min.

H¹—NMR δ (CDCl₃): 2.22 (s, 3H), 5.89 (brs, 2H), 7.25-7.38 (m, 6H), 7.41-7.53 (m, 3H), 8.03 (d, J=6 Hz, 1H).

### Example 102

### [3-Amino-2-(2-methylphenyl)-1-oxidopyridin-4-yl](3-fluorophenyl)methanone

Obtained as a yellow solid (73%) from the title compound of Example 101 following the procedure of Example 2.

LRMS (m/z): 323 (M+1)⁺.

Retention Time: 13 min.

H¹-NMR δ (CDCl₃): 2.22 (s, 3H), 6.26 (brs, 2H), 7.24-7.55 (m, 9H), 7.67 (d, J=6 Hz, 1H).

### Example 103

### [3-Amino-2-(2-methoxyphenyl)pyridin-4-yl](3-fluorophenyl)methanone

Obtained as a yellow solid (93%) from the title compound of Preparation 5 and 2-methoxyphenyl boronic acid following the experimental procedure described in Example 3 (4 h at 100° C.).

LRMS (m/z): 323 (M+1)⁺.

Retention Time: 14 min.

H¹-NMR δ (CDCl₃): 3.84 (s, 3H), 5.99 (brs, 2H), 7.04-7.16 (m, 2H), 7.25 (d, J=6 Hz, 1H), 7.28-7.52 (m, 6H), 8.05 (d, J=6 Hz, 1H).

### Example 104

### [3-Amino-2-(2-methoxyphenyl)-1-oxidopyridin-4-yl](3-fluorophenyl)methanone

Obtained as a yellow solid (78%) from the title compound of Example 103 following the procedure of Example 2 (4 h, 25° C.).

LRMS (m/z): 339 (M+1)⁺.

Retention Time: 13 min.

H¹—NMR δ (CDCl₃): 3.84 (s, 3H), 6.31 (brs, 2H), 7.10-7.21 (m, 2H), 7.25-7.59 (m, 7H), 7.65 (d, J=6 Hz, 1H).

### Example 105

### [3-Amino-2-(2-chlorophenyl)pyridin-4-yl](3-fluorophenyl)methanone

Obtained as a yellow solid (48%) from the title compound of Preparation 5 and 2-chlorophenyl boronic acid following the experimental procedure described in Example 3 (18 h at 100° C.).

LRMS (m/z): 327-329 (M+1)⁺.

Retention Time: 16 min.

H¹-NMR δ (CDCl₃): 5.87 (brs, 2H), 7.31 (d, J=4 Hz, 1H), 7.41-7.63 (m, 8H), 8.06 (d, J=6 Hz, 1H).

### Example 106

### [3-Amino-2-(2-chlorophenyl)-1-oxidopyridin-4-yl](3-fluorophenyl)methanone

Obtained as a yellow solid (64%) from the title compound of Example 105 following the procedure of Example 2.

LRMS (m/z): 343-345 (M+1)⁺.

Retention Time: 13 min.

H¹—NMR δ (CDCl₃): 6.24 (brs, 2H), 7.28-7.57 (m, 7H), 7.40 (d, J=6 Hz, 1H), 7.62-7.66 (m, 1H), 7.68 (d, J=6 Hz, 1H).

### Example 107

### [3-Amino-2-(2,6-difluorophenyl)pyridin-4-yl](3-fluorophenyl)methanone

Obtained as a yellow solid (52%) from the title compound of Preparation 5 and 1,3-difluorobenzene following the experimental procedure described in Example 33.

LRMS (m/z): 329 (M+1)⁺.

Retention Time: 15 min.

H¹—NMR δ (CDCl₃): 5.90 (brs, 2H), 7.05-7.13 (m, 2H), 7.26-7.54 (m, 5H), 7.34 (d, J=4 Hz, 1H), 8.11 (d, J=6 Hz, 1H).

### Example 108

### [3-Amino-2-(2,6-difluorophenyl)-1-oxidopyridin-4-yl](3-fluorophenyl)methanone

Obtained as a yellow solid (99%) from the title compound of Example 107 following the procedure of Example 2 (4 h, 25° C.).

LRMS (m/z): 345 (M+1)⁺.

Retention Time: 13 min.

H¹-NMR δ (CDCl₃): 6.35 (brs, 2H), 7.09-7.17 (m, 2H), 7.30-7.60 (m, 5H), 7.41 (d, J=6 Hz, 1H), 7.69 (d, J=6 Hz, 1H).

### Example 109

### [3-Amino-2-(1,3-benzodioxol-4-yl)pyridin-4-yl](3-fluorophenyl)methanone

Obtained as a yellow solid (82%) from the title compound of Preparation 5 and the title compound of Preparation 7 following the experimental procedure described in Example 3 (18 h at 100° C.).

LRMS (m/z): 337 (M+1)⁺.

Retention Time: 15 min.

H¹-NMR δ (CDCl₃): 6.06 (s, 2H), 6.16 (brs, 2H), 6.93 (dd, J=2 and 8 Hz, 1H), 7.01 (t, J=8 Hz, 1H), 7.09 (dd, J=2 and 8 Hz, 1H), 7.26 (d, J=6 Hz, 1H), 7.30 (m, 4H), 8.08 (d, J=6 Hz, 1H).

### Example 110

### [3-Amino-2-(1,3-benzodioxol-4-yl)-1-oxidopyridin-4-yl](3-fluorophenyl)methanone

Obtained as a yellow solid (43%) from the title compound of Example 109 following the procedure of Example 2.

LRMS (m/z): 353 (M+1)⁺.

Retention Time: 13 min.

H¹-NMR δ (CDCl₃): 6.06 (d, J=12 Hz, 1H), 6.07 (d, J=12 Hz, 1H), 6.48 (brs, 2H), 6.88-7.08 (m, 3H), 7.25-7.55 (m, 4H), 7.35 (d, J=6 Hz, 1H), 7.67 (d, J=8 Hz, 1H).

### Example 111

### [3-Amino-2-(2,6-dimethoxyphenyl)pyridin-4-yl](2,4-difluorophenyl)methanone

Obtained as a yellow solid (77%) from the title compound of Preparation 2e and 2,6-dimethoxyphenylboronic acid following the experimental procedure described in Example 3.

LRMS (m/z): 371 (M+1)⁺.

Retention Time: 13 min.

¹H-NMR δ (CDCl₃): 3.76 (s, 6H), 6.20 (brs, 2H), 6.70 (d, J=8 Hz, 2H), 6.90-7.07 (m, 2H), 7.10 (dd, J=2 and 4 Hz, 1H), 7.40 (t, J=8 Hz, 1H), 7.45-7.57 (m, 1H), 8.03 (d, J=4 Hz, 1H).

### Example 112

### [3-Amino-2-(2,6-dimethoxyphenyl)-1-oxidopyridin-4-yl](2,4-difluorophenyl)methanone

Obtained as a yellow solid (83%) from the title compound of Example 111 following the experimental procedure of Example 2.

LRMS (m/z): 387 (M+1)⁺.

Retention Time: 13 min.

¹H-NMR δ (CDCl₃): 3.80 (s, 6H), 6.46 (brs, 2H), 6.72 (d, J=8 Hz, 2H), 6.90-7.08 (m, 2H), 7.14 (dd, J=2 and 8 Hz, 1H), 7.43-7.51 (m, 2H), 7.60 (d, J=8 Hz, 1H).

### Example 113

### [3-Amino-2-(2-fluorophenyl)pyridin-4-yl](2,4-difluorophenyl)methanone

Obtained as a yellow solid (86%) from the title compound of Preparation 2e and 2-fluorophenylboronic acid following the experimental procedure described in Example 3.

LRMS (m/z): 329 (M+1)⁺.

¹H-NMR δ (CDCl₃): 6.30 (brs, 2H), 6.91-7.09 (m, 2H), 7.17 (dd, J=2 and 4 Hz, 1H), 7.20-7.36 (m, 2H), 7.40-7.59 (m, 3H), 8.04 (d, J=6 Hz, 1H).

### Example 114

### [3-Amino-2-(2-fluorophenyl)-1-oxidopyridin-4-yl](2,4-difluorophenyl)methanone

Obtained as a yellow solid (100%) from the title compound of Example 113 following the experimental procedure of Example 2.

LRMS (m/z): 345 (M+1)⁺.

¹H-NMR δ (CDCl₃): 6.51 (brs, 2H), 6.95-7.09 (m, 2H), 7.23-7.59 (m, 6H), 7.72 (d, J=6 Hz, 1H).

### Example 115

### [3-Amino-2-(2,6-dichlorophenyl)pyridin-4-yl](2-chlorophenyl)methanone

Obtained as a yellow solid (46%) from the title compound of Preparation 3e and 2,6-dichlorophenylboronic acid following the experimental procedure described in Example 5.

LRMS (m/z): 377, 379, 381, 383 (M+1)⁺.

¹H-NMR δ (CDCl₃): 6.18 (brs, 2H), 7.09 (d, J=4 Hz, 1H), 7.34-7.43 (m, 3H), 7.47-7.53 (m, 4H), 8.03 (d, J=4 Hz, 1H).

### Example 116

### [3-Amino-2-(2,6-dichlorophenyl)-1-oxidopyridin-4-yl](2-chlorophenyl)methanone

Obtained as a yellow solid (61%) from the title compound of Example 115 following the experimental procedure of Example 2.

LRMS (m/z): 393, 395, 397, 399 (M+1)⁺.

¹H-NMR δ (CDCl₃): 6.42 (brs, 2H), 7.13 (d, J=6 Hz, 1H), 7.40-7.57 (m, 7H), 7.61 (d, J=6 Hz, 1H).

### Example 117

### [3-Amino-2-(2,6-difluorophenyl)pyridin-4-yl](2-methoxyphenyl)methanone

Obtained as a yellow solid (60%) from the title compound of Preparation 11e and 1,3-difluorobenzene following the experimental procedure of Example 33.

LRMS (m/z): 341 (M+1)⁺.

Retention Time: 14 min.

¹H-NMR δ (CDCl₃): 3.80 (s, 3H), 6.24 (brs, 2H), 7.01-7.12 (m, 4H), 7.18 (d, J=6 Hz, 1H), 7.33 (dd, J=2 and 8 Hz, 1H), 7.37-7.54 (m, 2H), 8.01 (d, J=6 Hz, 1H).

### Example 118

### [3-Amino-2-(2,6-difluorophenyl)-1-oxidopyridin-4-yl](2-methoxyphenyl)methanone

Obtained as a yellow solid (71%) from the title compound of Example 117 following the experimental procedure of Example 2.

LRMS (m/z): 357 (M+1)⁺.

Retention Time: 13 min.

¹H-NMR δ (CDCl₃): 3.82 (s, 3H), 6.52 (brs, 2H), 7.01-7.23 (m, 5H), 7.32 (dd, J=2 and 8 Hz, 1H), 7.45-7.59 (m, 2H), 7.61 (d, J=6 Hz, 1H).

### Example 119

### [3-Amino-2-(2,6-dimethylphenyl)pyridin-4-yl](2-methoxyphenyl)methanone

Obtained as a yellow solid (50%) from the title compound of Preparation 11e and 2,6-dimethylphenylboronic acid following the experimental procedure described in Example 5.

LRMS (m/z): 333 (M+1)⁺.

Retention Time: 15 min.

¹H-NMR δ (CDCl₃): 2.08 (s, 6H), 3.80 (s, 3H), 6.09 (brs, 2H), 7.01-7.25 (m, 6H), 7.34 (dd, J=2 and 8 Hz, 1H), 7.45-7.54 (m, 1H), 7.98 (d, J=6 Hz, 1H).

### Example 120

### [3-Amino-2-(2,6-dimethylphenyl)-1-oxidopyridin-4-yl](2-methoxyphenyl)metha-none

Obtained as a yellow solid (70%) from the title compound of Example 119 following the experimental procedure of Example 2.

LRMS (m/z): 349 (M+1)⁺.

Retention Time: 14 min.

¹H-NMR δ (CDCl₃): 2.15 (s, 6H), 3.82 (s, 3H), 6.34 (brs, 2H), 7.01-7.12 (m, 2H), 7.17 (d, J=6 Hz, 1H), 7.20-7.24 (m, 2H), 7.30-7.36 (m, 2H), 7.45-7.54 (m, 1H), 7.61 (d, J=6 Hz, 1H).

### Example 121

### [3-Amino-2-(2-chlorophenyl)pyridin-4-yl](2-methoxyphenyl)methanone

Obtained as a yellow solid (60%) from the title compound of Preparation 11e and 2-chlorophenylboronic acid following the experimental procedure described in Example 3.

LRMS (m/z): 339 (M+1)⁺.

### Example 122

### [3-Amino-2-(2-chlorophenyl)-1-oxidopyridin-4-yl](2-methoxyphenyl)methanone

Obtained as a yellow solid (83%) from the title compound of Example 121 following the experimental procedure of Example 2.

LRMS (m/z): 355 (M+1)⁺.

Retention Time: 13 min.

¹H-NMR δ (CDCl₃): 3.82 (s, 3H), 6.39 (brs, 2H), 7.01-7.12 (m, 2H), 7.19 (d, J=6 Hz, 1H), 7.33 (dd, J=2 and 8 Hz, 1H), 7.38-7.54 (m, 4H), 7.60 (d, J=6 Hz, 1H), 7.60-7.66 (m, 1H).

### Example 123

### [3-Amino-2-(2-methoxyphenyl)pyridin-4-yl](2-methoxyphenyl)methanone

Obtained as a yellow solid (79%) from the title compound of Preparation 11e and 2-methoxyphenylboronic acid following the experimental procedure described in Example 3.

LRMS (m/z): 335 (M+1)⁺.

¹H-NMR δ (CDCl₃): 3.80 (s, 3H), 3.84 (s, 3H), 6.35 (brs, 2H), 7.00-7.15 (m, 4H), 7.08 (d, J=6 Hz, 1H), 7.29-7.52 (m, 4H), 7.93 (d, J=6 Hz, 1H).

### Example 124

### [3-Amino-2-(2-methoxyphenyl)-1-oxidopyridin-4-yl](2-methoxyphenyl)methanone

Obtained as a yellow solid (93%) from the title compound of Example 123 following the experimental procedure of Example 2.

LRMS (m/z): 351 (M+1)⁺.

Retention Time: 12 min.

¹H-NMR δ (CDCl₃): 3.82 (s, 3H), 3.84 (s, 3H), 6.47 (brs, 2H), 7.00-7.19 (m, 4H), 7.12 (d, J=6 Hz, 1H), 7.27-7.34 (m, 2H), 7.44-7.55 (m, 2H), 7.57 (d, J=6 Hz, 1H).

### Example 125

### [3-Amino-2-(2,6-difluorophenyl)pyridin-4-yl](2-chloro-4-fluorophenyl)metha-none

Obtained as a yellow solid (93%) from the title compound of Preparation 12e and 1,3-difluorobenzene following the experimental procedure of Example 33.

¹H-NMR δ (CDCl₃): 6.32 (brs, 2H), 7.03-7.18 (m, 4H), 7.27 (dd, J=2 and 8 Hz, 1H), 7.36-7.55 (m, 2H), 8.03 (d, J=6 Hz, 1H).

### Example 126

### [3-Amino-2-(2,6-difluorophenyl)-1-oxidopyridin-4-yl](2-chloro-4-fluorophenyl)methanone

Obtained as a yellow solid (98%) from the title compound of Example 125 following the experimental procedure of Example 2.

LRMS (m/z): 379 (M+1)⁺.

¹H-NMR δ (CDCl₃): 6.56 (brs, 2H), 7.10-7.20 (m, 4H), 7.27 (dd, J=2 and 8 Hz, 1H), 7.36-7.44 (m, 1H), 7.49-7.61 (m, 1H), 7.65 (d, J=8 Hz, 1H).

### Example 127

### [3-Amino-2-(2,6-dichlorophenyl)pyridin-4-yl](2-chloro-4-fluorophenyl)methanone

Obtained as a yellow solid (26%) from the title compound of Preparation 12e and 2,6-dichlorophenylboronic acid following the experimental procedure described in Example 5.

LRMS (m/z): 395, 397, 399, 401 (M+1)⁺.

¹H-NMR δ (CDCl₃): 6.16 (brs, 2H), 7.07 (d, J=6 Hz, 1H), 7.11-7.18 (m, 1H), 7.25-7.29 (m, 1H), 7.35-7.45 (m, 2H), 7.48-7.52 (m, 2H), 8.04 (d, J=6 Hz, 1H).

### Example 128

### [3-Amino-2-(2,6-dichlorophenyl)-1-oxidopyridin-4-yl](2-chloro-4-fluorophenyl)methanone

Obtained as a yellow solid (76%) from the title compound of Example 127 following the experimental procedure of Example 2.

¹H-NMR δ (CDCl₃): 6.40 (brs, 2H), 7.12 (d, J=8 Hz, 1H), 7.11-7.20 (m, 1H), 7.26 (dd, J=2 and 8 Hz, 1H), 7.39-7.57 (m, 4H), 7.63 (d, J=8 Hz, 1H).

### Example 129

### [3-Amino-2-(2-chlorophenyl)pyridin-4-yl](2-chloro-4-fluorophenyl)methanone

Obtained as a yellow solid (69%) from the title compound of Preparation 12e and 2-chlorophenylboronic acid following the experimental procedure described in Example 3.

LRMS (m/z): 361, 363, 365 (M+1)⁺.

### Example 130

### [3-Amino-2-(2-chlorophenyl)-1-oxidopyridin-4-yl](2-chloro-4-fluorophenyl)methanone

Obtained as a yellow solid (38%) from the title compound of Example 129 following the experimental procedure of Example 2.

LRMS (m/z): 377, 379, 381 (M+1)⁺.

### Composition Example 1

50,000 capsules each containing 100 mg of 3-amino-1-oxido-2-[2-(trifluoromethoxy)phenyl]pyridin-4-yl}(2,4-difluorophenyl)-methanone (active ingredient) were prepared according to the following formulation:

| | | |
|---|---|---|
| Active ingredient | 5 | Kg |
| Lactose monohydrate | 10 | Kg |
| Colloidal silicon dioxide | 0.1 | Kg |
| Corn starch | 1 | Kg |
| Magnesium stearate | 0.2 | Kg |

### Procedure

The above ingredients were sieved through a 60 mesh sieve, and were loaded into a suitable mixer and filled into 50,000 gelatine capsules.

### Composition Example 2

50,000 tablets each containing 50 mg of [3-amino-2-(2-hydroxyphenyl)-1-oxidopyridin-4-yl](2,4-difluorophenyl)methanone (active ingredient) were prepared from the following formulation:

| | | |
|---|---|---|
| Active ingredient | 2.5 | Kg |
| Microcrystalline cellulose | 1.95 | Kg |
| Spray dried lactose | 9.95 | Kg |
| Carboxymethyl starch | 0.4 | Kg |
| Sodium stearyl fumarate | 0.1 | Kg |
| Colloidal silicon dioxide | 0.1 | Kg |

### Procedure

All the powders were passed through a screen with an aperture of 0.6 mm, then mixed in a suitable mixer for 20 minutes and compressed into 300 mg tablets using 9 mm disc and flat bevelled punches. The disintegration time of the tablets was about 3 minutes.

1. A compound of formula (I) 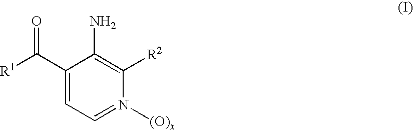
wherein:
R¹ represents a monocyclic or polycyclic, aryl or heteroaryl group optionally substituted by one, two or three substituents chosen from halogen atoms, straight or branched C₁₋₆ alkyl, hydroxy, straight or branched C₁₋₆ alkoxy, —SH, straight or branched C₁₋₆ alkylthio, nitro, cyano, —NR′R″, —CO₂R′, —C(O)—NR′R″, —N(R′″)C(O)—R′, and —N(R′″)—C(O)NR′R″, wherein R′, R″ and R′″ each independently represents a group chosen from a hydrogen atom and a straight or branched C₁₋₆ alkyl group or R′ and R″ together with the atom to which they are attached form a non-aromatic heterocyclic group;
R² represents a cyclic group chosen from aryl, heteroaryl, non-aromatic heterocyclic and carbocyclic groups, the cyclic groups being optionally substituted by one, two or three substituents chosen from halogen atoms, straight or branched C₁₋₆ alkyl, hydroxy, straight or branched C₁₋₆ alkoxy, —SH, straight or branched C₁₋₆ alkylthio, nitro, cyano, -trifluoromethyl, trifluoromethoxy, OR″, —NR′R″, —CO₂R′, —C(O)—NR′R″, —N(R″′)C(O)—R′, and —N(R′″)—C(O)NR′R″, wherein R′ and R′″ each independently represents a group chosen from a hydrogen atom and a straight or branched C₁₋₆ alkyl group and R″ represents a group of formula —(CH₂)ₙ—Y-G wherein n is an integer from 1 to 3; Y represents a group chosen a direct bond, —O— and —NR^{IV}—; wherein R^{IV} represents a group chosen from a hydrogen atom and a C₁₋₄ alkyl group; and G represents a group chosen from a hydrogen atom, a C₁₋₆ alkyl group and a non-aromatic nitrogen-containing heterocyclic ring bound to the group Y through its nitrogen atom, or R′ and R″ together with the atom to which they are attached form a non-aromatic heterocyclic group;
x has the value of zero or one;
or a pharmaceutically acceptable salt thereof.

2. A compound according to claim 1, wherein x has a value of 1.

3. A compound according to claim 1, wherein R¹ represents an optionally substituted monocyclic aryl or heteroaryl group.

4. A compound according to claim 3, wherein R¹ represents an optionally substituted phenyl group.

5. A compound according to claim 4, wherein the phenyl group is unsubstituted or substituted by 1 or 2 halogen atoms.

6. A compound according to claim 5, wherein the halogen atoms are independently chosen from chlorine and fluorine.

7. A compound according to claim 1, wherein R² represents a 5-10 membered cyclic group which is unsubstituted or substituted with 1, 2 or 3 substituents chosen from halogen atoms, C₁₋₄ alkyl groups, C₁₋₄ alkoxy groups, trifluoromethyl, trifluoromethoxy, —COOH, and groups of formula
—X—(CH₂)ₙ—Y-G
wherein X represents a group chosen from —C(O)NH—, —O— and —NH—; n is an integer from 1 to 3; Y represents a group chosen from direct bond, —O— and —NR^{IV}—; wherein R^{IV} represents a group chosen from a hydrogen atom and a C₁₋₄ alkyl group; and G represents a non-aromatic nitrogen-containing heterocyclic ring bound to the group Y through its nitrogen atom.

8. A compound according to claim 7, wherein the group R² is substituted with 1, 2 or 3 substituents and at least one of such substituents is in the ortho position with respect to the carbon atom through which R² is attached to the pyridine ring.

9. A compound according to claim 1, wherein the 5-10 membered cyclic group R² comprises from 0 to 3 heteroatoms chosen from nitrogen, oxygen and sulphur forming part of the ring system.

10. A compound according to claim 9, wherein the 5-10 membered cyclic group is chosen from the group consisting of phenyl, anthranyl, cyclohexyl, thienyl, furyl, pyridyl, benzodioxolyl and benzothienyl, all being optionally substituted.

11. A compound according to claim 1, wherein the cyclic group R² is unsubstituted or substituted with 1, 2 or 3 substituents chosen from halogen atoms and groups C₁₋₄ alkoxy, trifluoromethyl, trifluoromethoxy, —COOH, —C(O)O—C₁₋₄-alkyl, C₁₋₄-alkyl, morpholinylethoxy, methoxyethoxy, [(2-morpholin-4-ylethyl)amino]carbonyl, [(2-methoxyethyl)amino]carbonyl, and {2-[(dimethylamino)ethyl]amino}carbonyl.

12. A compound according to claim 1, wherein R¹ represents a phenyl group which is substituted by 1 or 2 halogen atoms independently chosen from chlorine and fluorine, R² represents a phenyl group which is substituted by 1 or 2 substituents chosen from chlorine, fluorine, methyl, methoxy and hydroxy, and at least one of such substituents is in the ortho position with respect to the carbon atom through which R² is attached to the pyridine.

13. A compound according to claim 12, wherein x has a value of 1.

14. A compound according to claim 1, which is one of:
(3-Amino-2-phenylpyridin-4-yl)(phenyl)methanone
(3-Amino-1-oxido-2-phenylpyridin-4-yl)(phenyl)methanone
[3-Amino-2-(2-methylphenyl)pyridin-4-yl](phenyl)methanone
[3-Amino-2-(2-methylphenyl)-1-oxidopyridin-4-yl](phenyl)methanone
[3-Amino-2-(2,6-dichlorophenyl)pyridin-4-yl](phenyl)methanone
[3-Amino-2-(2,6-dichlorophenyl)-1-oxidopyridin-4-yl](phenyl)methanone
[3-Amino-2-(2,6-difluoro-4-methoxyphenyl)pyridin-4-yl](phenyl)methanone
[3-Amino-2-(2,6-difluoro-4-methoxyphenyl)-1-oxidopyridin-4-yl](phenyl)methanone
[3-Amino-2-(4-chlorophenyl)pyridin-4-yl](phenyl)methanone
[3-Amino-2-(4-chlorophenyl)-1-oxidopyridin-4-yl](phenyl)methanone
(3-Amino-2-phenylpyridin-4-yl)(2,4-difluorophenyl)methanone
(3-Amino-1-oxido-2-phenylpyridin-4-yl)(2,4-difluorophenyl)methanone
[3-Amino-2-(2-hydroxyphenyl)pyridin-4-yl](2,4-difluorophenyl)methanone
[3-Amino-2-(2-hydroxyphenyl)-1-oxidopyridin-4-yl](2,4-difluorophenyl)methanone
[3-Amino-2-(2-methoxyphenyl)pyridin-4-yl](2,4-difluorophenyl)methanone
[3-Amino-2-(2-methoxyphenyl)-1-oxidopyridin-4-yl](2,4-difluorophenyl)methanone
{3-Amino-2-[2-(trifluoromethoxy)phenyl]pyridin-4-yl}(2,4-difluorophenyl)methanone
3-Amino-1-oxido-2-[2-(trifluoromethoxy)phenyl]pyridin-4-yl}(2,4-difluorophenyl)methanone
[3-Amino-2-(2-methylphenyl)pyridin-4-yl](2,4-difluorophenyl)methanone
[3-Amino-2-(2-methylphenyl)-1-oxidopyridin-4-yl](2,4-difluorophenyl)methanone
{3-Amino-2-[2-(trifluoromethyl)phenyl]pyridin-4-yl}(2,4-difluorophenyl)methanone
{3-Amino-1-oxido-2-[2-(trifluoromethyl)phenyl]pyridin-4-yl}(2,4-difluorophenyl)methanone
[3-Amino-2-(2-isopropylphenyl)pyridin-4-yl](2,4-difluorophenyl)methanone
[3-Amino-2-(2-isopropylphenyl)-1-oxidopyridin-4-yl](2,4-difluorophenyl)methanone
[3-Amino-2-(2-chlorophenyl)pyridin-4-yl](2,4-difluorophenyl)methanone
[3-Amino-2-(2-chlorophenyl)-1-oxidopyridin-4-yl](2,4-difluorophenyl)methanone
[3-Amino-2-(3-chlorophenyl)pyridin-4-yl](2,4-difluorophenyl)methanone
[3-Amino-2-(3-chlorophenyl)-1-oxidopyridin-4-yl](2,4-difluorophenyl)methanone
[3-Amino-2-(4-chlorophenyl)pyridin-4-yl](2,4-difluorophenyl)methanone
[3-Amino-2-(4-chlorophenyl)-1-oxidopyridin-4-yl](2,4-difluorophenyl)methanone
[3-Amino-2-(2,6-dichlorophenyl)pyridin-4-yl](2,4-difluorophenyl)methanone
[3-Amino-2-(2,6-dichlorophenyl)-1-oxidopyridin-4-yl](2,4-difluorophenyl)methanone
[3-Amino-2-(2,6-difluorophenyl)pyridin-4-yl](2,4-difluorophenyl)methanone
[3-Amino-2-(2,6-difluorophenyl)-1-oxidopyridin-4-yl](2,4-difluorophenyl)methanone
[3-Amino-2-(2,6-dimethylphenyl)pyridin-4-yl](2,4-difluorophenyl)methanone
[3-Amino-2-(2,6-dimethylphenyl)-1-oxidopyridin-4-yl](2,4-difluorophenyl)methanone
[3-Amino-2-(2,3-dimethoxyphenyl)pyridin-4-yl](2,4-difluorophenyl)methanone
[3-Amino-2-(2,3-dimethoxyphenyl)-1-oxidopyridin-4-yl](2,4-difluorophenyl)methanone
[3-Amino-2-(2,4-dichlorophenyl)pyridin-4-yl](2,4-difluorophenyl)methanone
[3-Amino-2-(2,4-dichlorophenyl)-1-oxidopyridin-4-yl](2,4-difluorophenyl)methanone
[3-Amino-2-(2-chloro-4-fluorophenyl)pyridin-4-yl](2,4-difluorophenyl)methanone
[3-Amino-2-(2-chloro-4-fluorophenyl)-1-oxidopyridin-4-yl](2,4-difluorophenyl)methanone
[3-Amino-2-(2,4-difluorophenyl)pyridin-4-yl](2,4-difluorophenyl)methanone
[3-Amino-2-(2,4-difluorophenyl)-1-oxidopyridin-4-yl](2,4-difluorophenyl)methanone
[3-Amino-2-(4-chloro-2-methylphenyl)pyridin-4-yl](2,4-difluorophenyl)methanone
[3-Amino-2-(4-chloro-2-methylphenyl)-1-oxidopyridin-4-yl](2,4-difluorophenyl)methanone
[3-Amino-2-(4-hydroxy-2-methylphenyl)pyridin-4-yl](2,4-difluorophenyl)methanone
[3-Amino-2-(4-hydroxy-2-methylphenyl)-1-oxidopyridin-4-yl](2,4-difluorophenyl)methanone
[3-Amino-2-(4-[2-(3-methylphenoxy)ethyl]morpholino)pyridin-4-yl](2,4-difluorophenyl)methanone
[3-Amino-2-(4-[2-(3-methylphenoxy)ethyl]morpholino)-1-oxidopyridin-4-yl](2,4-difluoro-phenyl)methanone
{3-Amino-2-[4-(2-methoxyethoxy)-2-methylphenyl]pyridin-4-yl}(2,4-difluorophenyl)methanone
{3-Amino-2-[4-(2-methoxyethoxy)-2-methylphenyl]-1-oxidopyridin-4-yl}(2,4-difluorophenyl)methanone
4-[3-Amino-4-(2,4-difluorobenzoyl)pyridin-2-yl]-3-methylbenzoic acid
4-[3-Amino-4-(2,4-difluorobenzoyl)-1-oxidopyridin-2-yl]-3-methylbenzoic acid
4-[3-Amino-4-(2,4-difluorobenzoyl)pyridin-2-yl]-3-methyl-N-(2-morpholin-4-ylethyl)benzamide
4-[3-Amino-4-(2,4-difluorobenzoyl)-1-oxidopyridin-2-yl]-3-methyl-N-(2-morpholin-4-ylethyl)benzamide
4-[3-Amino-4-(2,4-difluorobenzoyl)pyridin-2-yl]-3-methyl-N-(2-methoxyethyl)-3-methylbenzamide
4-[3-Amino-4-(2,4-difluorobenzoyl)-1-oxidopyridin-2-yl]-3-methyl-N-(2-methoxyethyl)-3-methylbenzamide
4-[3-Amino-4-(2,4-difluorobenzoyl)pyridin-2-yl]-3-methyl-N-[2-(dimethylamino)ethyl]-3-methylbenzamide
4-[3-Amino-4-(2,4-difluorobenzoyl)-1-oxidopyridin-2-yl]-3-methyl-N-[2-(dimethylamino)ethyl]-3-methylbenzamide
[3-Amino-2-(2,6-difluoro-4-methoxyphenyl)pyridin-4-yl](2,4-difluorophenyl)methanone
[3-Amino-2-(2,6-difluoro-4-methoxyphenyl)-1-oxidopyridin-4-yl](2,4-difluorophenyl)methanone
(3-Amino-3′-fluoro-2,4′-bipyridin-4-yl)(2,4-difluorophenyl)methanone
[3-Amino-2-(3-fluoropyridin-4-yl)-1-oxidopyridin-4-yl](2,4-difluorophenyl)methanone
(3-Amino-2,3′-bipyridin-4-yl)(2,4-difluorophenyl)methanone
(3-Amino-1-oxido-2-pyridin-3-ylpyridin-4-yl)(2,4-difluorophenyl)methanone
[3-Amino-2-(2-thienyl)pyridin-4-yl](2,4-difluorophenyl)methanone
[3-Amino-1-oxido-2-(2-thienyl)pyridin-4-yl](2,4-difluorophenyl)methanone
[3-Amino-2-(4-methyl-3-thienyl)pyridin-4-yl](2,4-difluorophenyl)methanone
[3-Amino-2-(4-methyl-3-thienyl)-1-oxidopyridin-4-yl](2,4-difluorophenyl)methanone
(3-Amino-2-cyclohexylpyridin-4-yl)(2,4-difluorophenyl)methanone
(3-Amino-2-cyclohexyl-1-oxidopyridin-4-yl)(2,4-difluorophenyl)methanone
[3-Amino-2-(1-naphthyl)pyridin-4-yl](2,4-difluorophenyl)methanone
[3-Amino-2-(1-naphthyl)-1-oxidopyridin-4-yl](2,4-difluorophenyl)methanone
[3-Amino-2-(2-ethoxy-1-naphthyl)pyridin-4-yl](2,4-difluorophenyl)methanone
[3-Amino-2-(2-ethoxy-1-naphthyl)-1-oxidopyridin-4-yl](2,4-difluorophenyl)-methanone
[3-Amino-2-(1-benzothien-3-yl)pyridin-4-yl](2,4-difluorophenyl)methanone
[3-Amino-2-(1-benzothien-3-yl)-1-oxidopyridin-4-yl](2,4-difluorophenyl)methanone
[3-Amino-2-(1,3-benzodioxol-4-yl)pyridin-4-yl](2,4-difluorophenyl)methanone
[3-Amino-2-(1,3-benzodioxol-4-yl)-1-oxidopyridin-4-yl](2,4-difluorophenyl)methanone
[3-Amino-2-(2-methylphenyl)pyridin-4-yl](2-chlorophenyl)methanone
[3-Amino-2-(2-methylphenyl)-1-oxidopyridin-4-yl](2-chlorophenyl)methanone
[3-Amino-2-(2-methoxyphenyl)pyridin-4-yl](2-chlorophenyl)methanone
[3-Amino-2-(2-methoxyphenyl)-1-oxidopyridin-4-yl](2-chlorophenyl)methanone
[3-Amino-2-(2-chlorophenyl)pyridin-4-yl](2-chlorophenyl)methanone
[3-Amino-2-(2-chlorophenyl)-1-oxidopyridin-4-yl](2-chlorophenyl)methanone
[3-Amino-2-(2,6-difluorophenyl)pyridin-4-yl](2-chlorophenyl)methanone
[3-Amino-2-(2,6-difluorophenyl)-1-oxidopyridin-4-yl](2-chlorophenyl)methanone
[3-Amino-2-(1,3-benzodioxol-4-yl)pyridin-4-yl](2-chlorophenyl)methanone
[3-Amino-2-(1,3-benzodioxol-4-yl)-1-oxidopyridin-4-yl](2-chlorophenyl)methanone
[3-Amino-2-(2-methylphenyl)pyridin-4-yl](3-methylphenyl)methanone
[3-Amino-2-(2-methylphenyl)-1-oxidopyridin-4-yl](3-methylphenyl)methanone
[3-Amino-2-(2-methoxyphenyl)pyridin-4-yl](3-methylphenyl)methanone
[3-Amino-2-(2-methoxyphenyl)-1-oxidopyridin-4-yl](3-methylphenyl)methanone
[3-Amino-2-(2-chlorophenyl)pyridin-4-yl](3-methylphenyl)methanone
[3-Amino-2-(2-chlorophenyl)-1-oxidopyridin-4-yl](3-methylphenyl)methanone
[3-Amino-2-(2,6-difluorophenyl)pyridin-4-yl](3-methylphenyl)methanone
[3-Amino-2-(2,6-difluorophenyl)-1-oxidopyridin-4-yl](3-methylphenyl)methanone
[3-Amino-2-(1,3-benzodioxol-4-yl)pyridin-4-yl](3-methylphenyl)methanone
[3-Amino-2-(1,3-benzodioxol-4-yl)-1-oxidopyridin-4-yl](3-methylphenyl)methanone
[3-Amino-2-(2-methylphenyl)pyridin-4-yl](3-fluorophenyl)methanone
[3-Amino-2-(2-methylphenyl)-1-oxidopyridin-4-yl](3-fluorophenyl)methanone
[3-Amino-2-(2-methoxyphenyl)pyridin-4-yl](3-fluorophenyl)methanone
[3-Amino-2-(2-methoxyphenyl)-1-oxidopyridin-4-yl](3-fluorophenyl)methanone
[3-Amino-2-(2-chlorophenyl)pyridin-4-yl](3-fluorophenyl)methanone
[3-Amino-2-(2-chlorophenyl)-1-oxidopyridin-4-yl](3-fluorophenyl)methanone
[3-Amino-2-(2,6-difluorophenyl)pyridin-4-yl](3-fluorophenyl)methanone
[3-Amino-2-(2,6-difluorophenyl)-1-oxidopyridin-4-yl](3-fluorophenyl)methanone
[3-Amino-2-(1,3-benzodioxol-4-yl)pyridin-4-yl](3-fluorophenyl)methanone
[3-Amino-2-(1,3-benzodioxol-4-yl)-1-oxidopyridin-4-yl](3-fluorophenyl)methanone
[3-Amino-2-(2,6-dimethoxyphenyl)pyridin-4-yl](2,4-difluorophenyl)methanone
[3-Amino-2-(2,6-dimethoxyphenyl)-1-oxidopyridin-4-yl](2,4-difluorophenyl)methanone
[3-Amino-2-(2-fluorophenyl)pyridin-4-yl](2,4-difluorophenyl)methanone
[3-Amino-2-(2-fluorophenyl)-1-oxidopyridin-4-yl](2,4-difluorophenyl)methanone
[3-Amino-2-(2,6-dichlorophenyl)pyridin-4-yl](2-chlorophenyl)methanone
[3-Amino-2-(2,6-dichlorophenyl)-1-oxidopyridin-4-yl](2-chlorophenyl)methanone
[3-Amino-2-(2,6-difluorophenyl)pyridin-4-yl](2-methoxyphenyl)methanone
[3-Amino-2-(2,6-difluorophenyl)-1-oxidopyridin-4-yl](2-methoxyphenyl)metha-none
[3-Amino-2-(2,6-dimethylphenyl)pyridin-4-yl](2-methoxyphenyl)methanone
[3-Amino-2-(2,6-dimethylphenyl)-1-oxidopyridin-4-yl](2-methoxyphenyl)metha-none
[3-Amino-2-(2-chlorophenyl)pyridin-4-yl](2-methoxyphenyl)methanone
[3-Amino-2-(2-chlorophenyl)-1-oxidopyridin-4-yl](2-methoxyphenyl)methanone
[3-Amino-2-(2-methoxyphenyl)pyridin-4-yl](2-methoxyphenyl)methanone
[3-Amino-2-(2-methoxyphenyl)-1-oxidopyridin-4-yl](2-methoxyphenyl)methanone
[3-Amino-2-(2,6-difluorophenyl)pyridin-4-yl](2-chloro-4-fluorophenyl)metha-none
[3-Amino-2-(2,6-difluorophenyl)-1-oxidopyridin-4-yl](2-chloro-4-fluorophenyl)methanone
[3-Amino-2-(2,6-dichlorophenyl)pyridin-4-yl](2-chloro-4-fluorophenyl)methanone
[3-Amino-2-(2,6-dichlorophenyl)-1-oxidopyridin-4-yl](2-chloro-4-fluorophenyl)methanone
[3-Amino-2-(2-chlorophenyl)pyridin-4-yl](2-chloro-4-fluorophenyl)methanone
[3-Amino-2-(2-chlorophenyl)-1-oxidopyridin-4-yl](2-chloro-4-fluorophenyl)methanone
and pharmaceutically acceptable salts thereof.

15. A compound according to claim 1, for use in the treatment of a pathological condition or disease susceptible to amelioration by inhibition of the p38 mitogen-activated protein kinase.

16. A pharmaceutical composition comprising a compound according to claim 1 in admixture with a pharmaceutically acceptable diluent or carrier.

17. (canceled)

18. (canceled)

19. A method for treating a subject afflicted with a pathological condition or disease susceptible to amelioration by inhibition of the p38 mitogen-activated protein kinase, which comprises administering to said subject an effective amount of a compound as defined in claim 1.

20. A method according to claim 19, wherein the pathological condition or disease is rheumatoid arthritis, ischemia-reperfusion injury, cerebral focal ischemia, acute coronary syndrome, COPD, Crohn's disease, irritable bowel syndrome, adult respiratory distress syndrome, osteoporosis, Alzheimer's disease, rheumatoid spondylitis, psoriasis, atherosclerosis, osteoarthritis or multiple myeloma.

21. A combination product comprising:
(i) a compound according to claim 1; and
(ii) another compound selected from (1) antagonists of M3 muscarinic receptors, (2) β2-agonists, (3) PDE4 inhibitors, (4) cortiocosteroids, (5) leukotriene D4 antagonists, (6) inhibitors of egfr-kinase, (7) antagonists of the A2B adenosine receptor, (8) NK1 receptor agonists, (9) CRTh2 antagonists, (10) syk kinase inhibitors, (11) CCR3 antagonists and (12) VLA-4 antagonists
for simultaneous, separate or sequential use in the treatment of the human or animal body.
